SEQUENCE LISTING (46 sequences)

SEQ ID NO: 1 - protein, 17 aa, Artificial Sequence
  androgen receptor peptide
KSTEDTAEYSPFKGGYT

SEQ ID NO: 2 - DNA, 89 bp, Artificial Sequence
  ADH1A amplicon region
tgctactgactggacgtacctggaagggagctattcttggtggctttaaaagtaaagaatgtgtcccaaaacttgtggctgattttatg

SEQ ID NO: 3 - DNA, 169 bp, Artificial Sequence
  ADH1B amplicon region
tcgtaggggtacctcctgcttcccagaacctctcaataaaccctatgctgctactgactggacgcacctggaagggggctgtttatggtggctttaagagtaaagaaggtatcccaaaacttgtggctgattttatggctaagaagttttcactggatgcgttaataac

SEQ ID NO: 4 - DNA, 389 bp, Artificial Sequence
  ADH1C amplicon region
aaccctatgctgctactgactggacgcacgtggaaaggagctatttttggaggctttaagagtaaagaatctgtccccaaacttgtggctgactttatggctaagaagttttcactggatgcattaataacaaatattttaccttttgaaaaaataaatgaaggatttgacctgcttcgctctggaaagagtatccgtaccgtcctgacgttttgaaacaatacagatgccttcccttgtagcagttttcagcctcctctaccctacatgatctggagcaacagctaggaaatatcattaattctgctcttcagagatgttaaaaataaattacacgtgggagctttccaaagaaatggaaattgatgggaaattatttgtcaagcaaa

SEQ ID NO: 5 - DNA, 68 bp, Artificial Sequence
  ADH4 amplicon region
ctggatatggcgctgctgttaaaactggcaaggtcaaacctggttccacttgcgtcgtctttggcctg

SEQ ID NO: 6 - DNA, 129 bp, Artificial Sequence
  HNF1B amplicon region
aaggagctgcaggcgctcaacaccgaggaggcggcggagcagcgggcggaggtggaccggatgctcagtgaggacccttggagggctgctaaaatgatcaagggttacatgcagcaacacaacatcccc

SEQ ID NO: 7 - DNA, 137 bp, Artificial Sequence
  Androgen receptor amplicon region
attccgaaggaaaaattgtccatcttgtcgtcttcggaaatgttatgaagcagggatgactctgggagcccggaagctgaagaaacttggtaatctgaaactacaggaggaaggagaggcttccagcaccaccagcc

SEQ ID NO: 8 - DNA, 69 bp, Artificial Sequence
  Prostate specific antigen amplicon region
gtcctcacagctgcccactgcatcaggaagccaggtgatgactccagccacgacctcatgctgctccgc

SEQ ID NO: 9 - DNA, 67 bp, Artificial Sequence
  Cytokeratin amplicon region
attacttcaagatcatcgaggacctgagggctcagatcttcgcaaatactgtggacaatgcccgcat

SEQ ID NO: 10 - DNA, 22 bp, Artificial Sequence
  ADH1A forward primer
cgtacctggaagggagctattc

SEQ ID NO: 11 - DNA, 20 bp, Artificial Sequence
  ADH1A reverse primer
gccacaagttttgggacaca

SEQ ID NO: 12 - DNA, 16 bp, Artificial Sequence
  ADH1A probe
tggtggctttaaaagt

SEQ ID NO: 13 - DNA, 19 bp, Artificial Sequence
  ADH1B forward primer
tgactggacgcacctggaa

SEQ ID NO: 14 - DNA, 24 bp, Artificial Sequence
  ADH1B reverse primer
gttattaacgcatccagtgaaaac

SEQ ID NO: 15 - DNA, 21 bp, Artificial Sequence
  ADH1B probe
ttatggtggctttaagagtaa

SEQ ID NO: 16 - DNA, 25 bp, Artificial Sequence
  ADH1C forward primer
aaggagctatttttggaggctttaa

SEQ ID NO: 17 - DNA, 23 bp, Artificial Sequence
  ADH1C reverse primer
agaggaggctgaaaactgctaca

SEQ ID NO: 18 - DNA, 21 bp, Artificial Sequence
  ADH1C probe
ttatggtggctttaagagtaa

SEQ ID NO: 19 - DNA, 21 bp, Artificial Sequence
  ADH4 forward primer
ggatatggcgctgctgttaaa

SEQ ID NO: 20 - DNA, 19 bp, Artificial Sequence
  ADH4 reverse primer
gacgacgcaagtggaacca

SEQ ID NO: 21 - DNA, 15 bp, Artificial Sequence
  ADH4 probe
ctggcaaggtcaaac

SEQ ID NO: 22 - DNA, 17 bp, Artificial Sequence
  HNF1B forward primer
aacaccgaggaggcggc

SEQ ID NO: 23 - DNA, 25 bp, Artificial Sequence
  HNF1B reverse primer
ctgcatgtaacccttgatcatttta

SEQ ID NO: 24 - DNA, 17 bp, Artificial Sequence
  HNF1B probe
gaccggatgctcagtga

SEQ ID NO: 25 - DNA, 23 bp, Artificial Sequence
  Androgen receptor forward primer
tgtcgtcttcggaaatgttatga

SEQ ID NO: 26 - DNA, 20 bp, Artificial Sequence
  Androgen receptor reverse primer
ggaagcctctccttcctcct

SEQ ID NO: 27 - DNA, 15 bp, Artificial Sequence
  Androgen receptor probe
actctgggagcccgg

SEQ ID NO: 28 - DNA, 18 bp, Artificial Sequence
  Prostate specific antigen forward primer
acagctgcccactgcatc

SEQ ID NO: 29 - DNA, 17 bp, Artificial Sequence
  Prostate specific antigen reverse primer
aggcggagcagcatgag

SEQ ID NO: 30 - DNA, 15 bp, Artificial Sequence
  Prostate specific antigen probe
ccaggtgatgactcc

SEQ ID NO: 31 - DNA, 25 bp, Artificial Sequence
  Cytokeratin forward primer
ccattacttcaagatcatcgaggac

SEQ ID NO: 32 - DNA, 22 bp, Artificial Sequence
  Cytokeratin reverse primer
gggcattgtccacagtatttgc

SEQ ID NO: 33 - DNA, 15 bp, Artificial Sequence
  Cytokeratin probe
tgagggctcagatct

SEQ ID NO: 34 - DNA, 25 bp, Artificial Sequence
  ADH4 probe
aactggcaaggtcaaacctggttcc

SEQ ID NO: 35 - DNA, 25 bp, Artificial Sequence
  ADH1B probe
gggggctgtttatggtggctttaag

SEQ ID NO: 36 - DNA, 25 bp, Artificial Sequence
  ADH1C probe
tggaaagagtatccgtaccgtcctg

SEQ ID NO: 37 - protein, 375 aa, Homo sapiens
MSTAGKVIKCKAAVLWEVKKPFSIEDVEVAPPKAYEVRIKMVAVGICHTDDHVVSGNLVTPLPVILGHEAAGIVESVGEGVTTVKPGDKVIPLFTPQCGKCRVCKNPESNYCLKNDLGNPRGTLQDGTRRFTCRGKPIHHFLGTSTFSQYTVVDENAVAKIDAASPLEKVCLIGCGFSTGYGSAVNVAKVTPGSTCAVFGLGGVGLSAVMGCKAAGAARIIAVDINKDKFAKAKELGATECINPQDYKKPIQEVLKEMTDGGVDFSFEVIGRLDTMMASLLCCHEACGTSVIVGVPPASQNLSINPMLLLTGRTWKGAVYGGFKSKEGIPKLVADFMAKKFSLDALITHVLPFEKINEGFDLLHSGKSIRTVLTF

SEQ ID NO: 38 - DNA, 2689 bp, Homo sapiens
aatatatctgctttatgcactcaagcagagaagaaatccacaaagactcacagtctgctggtgggcagagaagacagaaacgacatgagcacagcaggaaaagtaatcaaatgcaaagcagctgtgctatgggaggtaaagaaacccttttccattgaggatgtggaggttgcacctcctaaggcttatgaagttcgcattaagatggtggctgtaggaatctgtcacacagatgaccacgtggttagtggcaacctggtgaccccccttcctgtgattttaggccatgaggcagccggcatcgtggagagtgttggagaaggggtgactacagtcaaaccaggtgataaagtcatcccgctctttactcctcagtgtggaaaatgcagagtttgtaaaaacccggagagcaactactgcttgaaaaatgatctaggcaatcctcgggggaccctgcaggatggcaccaggaggttcacctgcagggggaagcccattcaccacttccttggcaccagcaccttctcccagtacacggtggtggatgagaatgcagtggccaaaattgatgcagcctcgcccctggagaaagtctgcctcattggctgtggattctcgactggttatgggtctgcagttaacgttgccaaggtcaccccaggctctacctgtgctgtgtttggcctgggaggggtcggcctatctgctgttatgggctgtaaagcagctggagcagccagaatcattgcggtggacatcaacaaggacaaatttgcaaaggccaaagagttgggtgccactgaatgcatcaaccctcaagactacaagaaacccattcaggaagtgctaaaggaaatgactgatggaggtgtggatttttcgtttgaagtcatcggtcggcttgacaccatgatggcttccctgttatgttgtcatgaggcatgtggcacaagcgtcatcgtaggggtacctcctgcttcccagaacctctcaataaaccctatgctgctactgactggacgcacctggaagggggctgtttatggtggctttaagagtaaagaaggtatcccaaaacttgtggctgattttatggctaagaagttttcactggatgcgttaataacccatgttttaccttttgaaaaaataaatgaaggatttgacctgcttcactctgggaaaagtatccgtaccgtcctgacgttttgaggcaatagagatgccttcccctgtagcagtcttcagcctcctctaccctacaagatctggagcaacagctaggaaatatcattaattcagctcttcagagatgttatcaataaattacacatgggggctttccaaagaaatggaaattgatgggaaattatttttcaggaaaatttaaaattcaagtgagaagtaaataaagtgttgaacatcagctggggaattgaagccaacaaaccttccttcttaaccattctactgtgtcacctttgccattgaggaaaaatattcctgtgacttcttgcatttttggtatcttcataatctttagtcatcgaatcccagtggaggggacccttttacttgccctgaacatacacatgctgggccattgtgattgaagtcttctaactctgtctcagttttcactgtcgacattttcctttttctaataaaaatgtaccaaatccctggggtaaaagctagggtaaggtaaaggatagactcacatttacaagtagtgaaggtccaagagttctaaatacaggaaatttcttaggaactcaaataaaatgccccacattttactacagtaaatggcagtgtttttatgacttttatactatttctttatggtcgatatacaattgattttttaaaataatagcagatttcttgcttcatatgacaaagcctcaattactaattgtaaaaactgaactattcccagaatcatgttcaaaaaatctgtaatttttgctgatgaaagtgcttcattgactaaacagtattagtttgtggctataaatgattatttagatgatgactgaaaatgtgtataaagtaattaaaagtaatatggtggctttaagtgtagagatgggatggcaaatgctgtgaatgcagaatgtaaaattggtaactaagaaatggcacaaacaccttaagcaatatattttcctagtagatatatatatacacatacatatatacacatatacaaatgtatatttttgcaaaattgttttcaatctagaacttttctattaactaccatgtcttaaaatcaagtctataatcctagcattagtttaatattttgaatatgtaaagacctgtgttaatgctttgttaatgcttttcccactctcatttgttaatgctttcccactctcaggggaaggatttgcattttgagctttatctctaaatgtgacatgcaaagattattcctggtaaaggaggtagctgtctccaaaaatgctattgttgcaatatctacattctatttcatattatgaaagaccttagacataaagtaaaatagtttatcatttactgtgtgatcttcagtaagtctctcaggctctctgagcttgttcatcctttgttttgaaaaaattactcaaccaatccattacagcttaaccaagattaaatgggatgatgttaatgaaagagcttcgccaaaaaaaaa

SEQ ID NO: 39 - protein, 375 aa, Homo sapiens
MSTAGKVIKCKAAVLWELKKPFSIEEVEVAPPKAHEVRIKMVAAGICRSDEHVVSGNLVTPLPVILGHEAAGIVESVGEGVTTVKPGDKVIPLFTPQCGKCRICKNPESNYCLKNDLGNPRGTLQDGTRRFTCSGKPIHHFVGVSTFSQYTVVDENAVAKIDAASPLEKVCLIGCGFSTGYGSAVKVAKVTPGSTCAVFGLGGVGLSVVMGCKAAGAARIIAVDINKDKFAKAKELGATECINPQDYKKPIQEVLKEMTDGGVDFSFEVIGRLDTMMASLLCCHEACGTSVIVGVPPDSQNLSINPMLLLTGRTWKGAIFGGFKSKESVPKLVADFMAKKFSLDALITNILPFEKINEGFDLLRSGKSIRTVLTF

SEQ ID NO: 40 - DNA, 1497 bp, Homo sapiens
aacatacctgctttatgcactcaagcagagaagaaatccacaagtactcaccagcctcctggtctgcagagaagacagaatcaatatgagcacagcaggaaaagtaatcaaatgcaaagcagctgtgctatgggagttaaagaaacccttttccattgaggaggtagaggttgcacctcctaaggctcatgaagttcgcattaagatggtggctgcaggaatctgtcgttcagatgagcatgtggttagtggcaacctggtgaccccccttcctgtgattttaggccatgaggcagccggcatcgtggaaagtgttggagaaggggtgactacagtcaaaccaggtgataaagtcatcccgctctttactcctcagtgtggaaaatgcagaatttgtaaaaacccagaaagcaactactgcttgaaaaatgatctaggcaatcctcgggggaccctgcaggatggcaccaggaggttcacctgcagcgggaagcccatccaccacttcgtcggcgtcagcaccttctcccagtacacagtggtggatgagaatgcagtggccaaaattgatgcagcctcgcccctggagaaagtctgcctcattggctgtggattttcgactggttatgggtctgcagtcaaagttgccaaggtcaccccagggtctacctgtgctgtgtttggcctgggaggggtcggcctatctgttgttatgggctgtaaagcagctggagcagccagaatcattgctgtggacatcaacaaggacaaatttgcaaaggctaaagagttgggtgccactgaatgcatcaaccctcaagactacaagaaacccattcaggaagtgctaaaggaaatgactgatggaggtgtggatttttcgtttgaagtcatcggtcggcttgacaccatgatggcttccctgttatgttgtcatgaggcatgtggcacaagtgtcattgtaggggtacctcctgattcccagaacctctcaataaaccctatgctgctactgactggacgcacgtggaaaggagctatttttggaggctttaagagtaaagaatctgtccccaaacttgtggctgactttatggctaagaagttttcactggatgcattaataacaaatattttaccttttgaaaaaataaatgaaggatttgacctgcttcgctctggaaagagtatccgtaccgtcctgacgttttgaaacaatacagatgccttcccttgtagcagttttcagcctcctctaccctacatgatctggagcaacagctaggaaatatcattaattctgctcttcagagatgttaaaaataaattacacgtgggagctttccaaagaaatggaaattgatgggaaattatttgtcaagcaaatgtttaaaatccaaatgagaactaaataaagtgttgaacatcaactggggaattgaagccaataaaccttccttcttaaccattcaaaaaaaaaaaaaaaaaaaaaaaaaa

SEQ ID NO: 41 - protein, 394 aa, Homo sapiens
MKCLVSRHTVRETLDMVFQRMSVEAGVIKCKAAVLWEQKQPFSIEEIEVAPPKTKEVRIKILATGICRTDDHVIKGTMVSKFPVIVGHEATGIVESIGEGVTTVKPGDKVIPLFLPQCRECNACRNPDGNLCIRSDITGRGVLADGTTRFTCKGKPVHHFMNTSTFTEYTVVDESSVAKIDDAAPPEKVCLIGCGFSTGYGAAVKTGKVKPGSTCVVFGLGGVGLSVIMGCKSAGASRIIGIDLNKDKFEKAMAVGATECISPKDSTKPISEVLSEMTGNNVGYTFEVIGHLETMIDALASCHMNYGTSVVVGVPPSAKMLTYDPMLLFTGRTWKGCVFGGLKSRDDVPKLVTEFLAKKFDLDQLITHVLPFKKISEGFELLNSGQSIRTVLTF

SEQ ID NO: 42 - protein, 386 aa, Homo sapiens
MFAEIQIQDKDRMGTAGKVIKCKAAVLWEQKQPFSIEEIEVAPPKTKEVRIKILATGICRTDDHVIKGTMVSKFPVIVGHEATGIVESIGEGVTTVKPGDKVIPLFLPQCRECNACRNPDGNLCIRSDITGRGVLADGTTRFTCKGKPVHHFMNTSTFTEYTVVDESSVAKIDDAAPPEKVCLIGCGFSTGYGAAVKTGKVKPGSTCVVFGLGGVGLSVIMGCKSAGASRIIGIDLNKDKFEKAMAVGATECISPKDSTKPISEVLSEMTGNNVGYTFEVIGHLETMIDALASCHMNYGTSVVVGVPPSAKMLTYDPMLLFTGRTWKGCVFGGLKSRDDVPKLVTEFLAKKFDLDQLITHVLPFKKISEGFELLNSGQSIRTVLTF

SEQ ID NO: 43 - DNA, 2120 bp, Homo sapiens
aattaaacacttggagatattccttgaggaatgaaatgcttggtgagcaggcatacagtgagggaaacactggatatggtgtttcagagaatgtcagtggaagcaggggttattaaatgcaaagcagctgtgctttgggagcagaagcaacccttctccattgaggaaatagaagttgccccaccaaagactaaagaagttcgcattaagattttggccacaggaatctgtcgcacagatgaccatgtgataaaaggaacaatggtgtccaagtttccagtgattgtgggacatgaggcaactgggattgtagagagcattggagaaggagtgactacagtgaaaccaggtgacaaagtcatccctctctttctgccacaatgtagagaatgcaatgcttgtcgcaacccagatggcaacctttgcattaggagcgatattactggtcgtggagtactggctgatggcaccaccagatttacatgcaagggcaaaccagtccaccacttcatgaacaccagtacatttaccgagtacacagtggtggatgaatcttctgttgctaagattgatgatgcagctcctcctgagaaagtctgtttaattggctgtgggttttccactggatatggcgctgctgttaaaactggcaaggtcaaacctggttccacttgcgtcgtctttggcctgggaggagttggcctgtcagtcatcatgggctgtaagtcagctggtgcatctaggatcattgggattgacctcaacaaagacaaatttgagaaggccatggctgtaggtgccactgagtgtatcagtcccaaggactctaccaaacccatcagtgaggtgctgtcagaaatgacaggcaacaacgtgggatacacctttgaagttattgggcatcttgaaaccatgattgatgccctggcatcctgccacatgaactatgggaccagcgtggttgtaggagttcctccatcagccaagatgctcacctatgacccgatgttgctcttcactggacgcacatggaagggatgtgtctttggaggtttgaaaagcagagatgatgtcccaaaactagtgactgagttcctggcaaagaaatttgacctggaccagttgataactcatgttttaccatttaaaaaaatcagtgaaggatttgagctgctcaattcaggacaaagcattcgaacggtcctgacgttttgagatccaaagtggcaggaggtctgtgttgtcatggtgaactggagtttctcttgtgagagttccctcatctgaaatcatgtatctgtctcacaaatacaagcataagtagaagatttgttgaagacatagaacccttataaagaattattaacctttataaacatttaaagtcttgtgagcacctgggaattagtataataacaatgttaatatttttgatttacattttgtaaggctataattgtatcttttaagaaaacatacacttggatttctatgttgaaatggagatttttaagagttttaaccagctgctgcagatatataactcaaaacagatatagcgtataaagatatagtaaatgcatctcctagagtaatattcacttaacacattgaaactattattttttagatttgaatataaatgtattttttaaacacttgttatgagttaagttggattacattttgaaatcagttcattccatgatgcatattactggattagattaagaaagacagaaaagattaagggacgggcacatttttcaacgattaagaatcatcattacataacttggtgaaactgaaaaagtatatcatatgggtacacaaggctatttgccagcatatattaatattttagaaaatattccttttgtaatactgaatataaacatagagctagaatcatattatcatacttatcataatgttcaatttgatacagtagaattgcaagtccctaagtccctattcactgtgcttagtagtgactccatttaataaaaagtgtttttagtttttaacaactacactgatgtatctatatatatctataacatgttaaaaattcttaagaaaattaaaaattatataaaatgaaaaaaaaaaaaaaaaaa

SEQ ID NO: 44 - DNA, 2307 bp, Homo sapiens
tttcaatagctttgattatcttgtttttgttagatccctcctcttggtttgatcatagtagttactgtatttctttttataagttggtctgcaaagggtagggcttgcagaccattgcaaagttgtgacggctgtgagtcatattgctgaaggtggaactctgaagccagactatctatgtgaaggcacaagctgctgttatatacaacagagtgaactgagcatcagtcagaaaaagtctatgtttgcagaaatacagatccaagacaaagacaggatgggcactgctggaaaagttattaaatgcaaagcagctgtgctttgggagcagaagcaacccttctccattgaggaaatagaagttgccccaccaaagactaaagaagttcgcattaagattttggccacaggaatctgtcgcacagatgaccatgtgataaaaggaacaatggtgtccaagtttccagtgattgtgggacatgaggcaactgggattgtagagagcattggagaaggagtgactacagtgaaaccaggtgacaaagtcatccctctctttctgccacaatgtagagaatgcaatgcttgtcgcaacccagatggcaacctttgcattaggagcgatattactggtcgtggagtactggctgatggcaccaccagatttacatgcaagggcaaaccagtccaccacttcatgaacaccagtacatttaccgagtacacagtggtggatgaatcttctgttgctaagattgatgatgcagctcctcctgagaaagtctgtttaattggctgtgggttttccactggatatggcgctgctgttaaaactggcaaggtcaaacctggttccacttgcgtcgtctttggcctgggaggagttggcctgtcagtcatcatgggctgtaagtcagctggtgcatctaggatcattgggattgacctcaacaaagacaaatttgagaaggccatggctgtaggtgccactgagtgtatcagtcccaaggactctaccaaacccatcagtgaggtgctgtcagaaatgacaggcaacaacgtgggatacacctttgaagttattgggcatcttgaaaccatgattgatgccctggcatcctgccacatgaactatgggaccagcgtggttgtaggagttcctccatcagccaagatgctcacctatgacccgatgttgctcttcactggacgcacatggaagggatgtgtctttggaggtttgaaaagcagagatgatgtcccaaaactagtgactgagttcctggcaaagaaatttgacctggaccagttgataactcatgttttaccatttaaaaaaatcagtgaaggatttgagctgctcaattcaggacaaagcattcgaacggtcctgacgttttgagatccaaagtggcaggaggtctgtgttgtcatggtgaactggagtttctcttgtgagagttccctcatctgaaatcatgtatctgtctcacaaatacaagcataagtagaagatttgttgaagacatagaacccttataaagaattattaacctttataaacatttaaagtcttgtgagcacctgggaattagtataataacaatgttaatatttttgatttacattttgtaaggctataattgtatcttttaagaaaacatacacttggatttctatgttgaaatggagatttttaagagttttaaccagctgctgcagatatataactcaaaacagatatagcgtataaagatatagtaaatgcatctcctagagtaatattcacttaacacattgaaactattattttttagatttgaatataaatgtattttttaaacacttgttatgagttaagttggattacattttgaaatcagttcattccatgatgcatattactggattagattaagaaagacagaaaagattaagggacgggcacatttttcaacgattaagaatcatcattacataacttggtgaaactgaaaaagtatatcatatgggtacacaaggctatttgccagcatatattaatattttagaaaatattccttttgtaatactgaatataaacatagagctagaatcatattatcatacttatcataatgttcaatttgatacagtagaattgcaagtccctaagtccctattcactgtgcttagtagtgactccatttaataaaaagtgtttttagtttttaacaactacactgatgtatctatatatatctataacatgttaaaaattcttaagaaaattaaaaattatataaaatgaaaaaaaaaaaaaaaaaa

SEQ ID NO: 45 - protein, 557 aa, Homo sapiens
MVSKLTSLQQELLSALLSSGVTKEVLVQALEELLPSPNFGVKLETLPLSPGSGAEPDTKPVFHTLTNGHAKGRLSGDEGSEDGDDYDTPPILKELQALNTEEAAEQRAEVDRMLSEDPWRAAKMIKGYMQQHNIPQREVVDVTGLNQSHLSQHLNKGTPMKTQKRAALYTWYVRKQREILRQFNQTVQSSGNMTDKSSQDQLLFLFPEFSQQSHGPGQSDDACSEPTNKKMRRNRFKWGPASQQILYQAYDRQKNPSKEEREALVEECNRAECLQRGVSPSKAHGLGSNLVTEVRVYNWFANRRKEEAFRQKLAMDAYSSNQTHSLNPLLSHGSPHHQPSSSPPNKLSGVRYSQQGNNEITSSSTISHHGNSAMVTSQSVLQQVSPASLDPGHNLLSPDGKMISVSGGGLPPVSTLTNIHSLSHHNPQQSQNLIMTPLSGVMAIAQSLNTSQAQSVPVINSVAGSLAALQPVQFSQQLHSPHQQPLMQQSPGSHMAQQPFMAAVTQLQNSHMYAHKQEPPQYSHTSRFPSAMVVTDTSSISTLTNMSSSKQCPLQAW

SEQ ID NO: 46 - DNA, 2842 bp, Homo sapiens
aatttgcatatcttatatggcctaatggtggcgatcatggcaagttagaagttttctgactcctttcggaggagcctccgggaccccggggagtaacaggtgtctggaggctgaagggtggaggggttcctggatttggggtttgcttgtgaaactcccctccaccctcctctctcgcacccacccaccccctcacccccttctttttccgtccttggaaaatggtgtccaagctcacgtcgctccagcaagaactcctgagcgccctgctgagctccggggtcaccaaggaggtgctggttcaggccttggaggagttgctgccatccccgaacttcggggtgaagctggagacgctgcccctgtcccctggcagcggggccgagcccgacaccaagccggtcttccatactctcaccaacggccacgccaagggccgcttgtccggcgacgagggctccgaggacggcgacgactatgacacacctcccatcctcaaggagctgcaggcgctcaacaccgaggaggcggcggagcagcgggcggaggtggaccggatgctcagtgaggacccttggagggctgctaaaatgatcaagggttacatgcagcaacacaacatcccccagagggaggtggtcgatgtcaccggcctgaaccagtcgcacctctcccagcatctcaacaagggcacccctatgaagacccagaagcgtgccgctctgtacacctggtacgtcagaaagcaacgagagatcctccgacaattcaaccagacagtccagagttctggaaatatgacagacaaaagcagtcaggatcagctgctgtttctctttccagagttcagtcaacagagccatgggcctgggcagtccgatgatgcctgctctgagcccaccaacaagaagatgcgccgcaaccggttcaaatgggggcccgcgtcccagcaaatcttgtaccaggcctacgatcggcaaaagaaccccagcaaggaagagagagaggccttagtggaggaatgcaacagggcagaatgtttgcagcgaggggtgtccccctccaaagcccacggcctgggctccaacttggtcactgaggtccgtgtctacaactggtttgcaaaccgcaggaaggaggaggcattccggcaaaagctggccatggacgcctatagctccaaccagactcacagcctgaaccctctgctctcccacggctccccccaccaccagcccagctcctctcctccaaacaagctgtcaggagtgcgctacagccagcagggaaacaatgagatcacttcctcctcaacaatcagtcaccatggcaacagcgccatggtgaccagccagtcggttttacagcaagtctccccagccagcctggacccaggccacaatctcctctcacctgatggtaaaatgatctcagtctcaggaggaggtttgcccccagtcagcaccttgacgaatatccacagcctctcccaccataatccccagcaatctcaaaacctcatcatgacacccctctctggagtcatggcaattgcacaaagcctcaacacctcccaagcacagagtgtccctgtcatcaacagtgtggccggcagcctggcagccctgcagcccgtccagttctcccagcagctgcacagccctcaccagcagcccctcatgcagcagagcccaggcagccacatggcccagcagcccttcatggcagctgtgactcagctgcagaactcacacatgtacgcacacaagcaggaacccccccagtattcccacacctcccggtttccatctgcaatggtggtcacagataccagcagcatcagtacactcaccaacatgtcttcaagtaaacagtgtcctctacaagcctggtgatgcccacacaccacttacttcgtgcgcaacaacaaggaccctgttttccacaccatcaccctctgggcagctgtcatggaaaagcccagtgacctgaccggcacctgcgagaggtccctgcttacctgacggacgtcctgctggcacctcagacaatccactctcaggaggcgcagcccgaagcccagtttcccttctatgcagtattgccacaatgcctctcccacgatgtcaaggactcctgtctgtcctggaggtgggagacaaggaaccaccgaagaggaagcaagaaagccgtactgtctatgttgtgatccttcatcgaacaaactgatgcgaaaacttgaatctgttactgaaatgaggagagaaggacatgtgctattgaactgagccaaacacactgtaaatatccacagactccctcccctgcccccatcccacatgatcttgagatttcttttaaagaagtaaatttgtccaatggctgtaaactataaactactgtaattaagtgcaatttcccctctgtgtcctctcccctctgccctgtatataatactaaagtgtctattagttttctttgtaaaggtcagagtcaaaatttcaaaagtgatctgtcccctctcccctcatggagaaacatcctaagtgggaagtgaagccccttgtcctctcccgcgggcctggacacttatggggacagcataccttggactgactaccagctaactccagtctcctgacattaagacacacctctggatccctggaggggctgaatgtagtgtgtcagagtaacatgccagcttcctgtgggccaggagctcagccgtgcactccctaagaaaccccagggcagggaaactggctgtttgatagcagaagaaaaagttgcagtctcagaaagccttccattaaaacaatttattttatcactaaaaaaa

(19) United States
(12) Patent Application Publication
(10) Pub. No.: US 2016/0010157 A1
(43) Pub. Date: Jan. 14, 2016

(54) METHODS AND COMPOSITIONS RELATING TO PROLIFERATIVE DISORDERS OF THE PROSTATE

(71) Applicant: WEST CHESTER UNIVERSITY OF PENNSYLVANIA, West Chester, PA (US)

(72) Inventors: Leslie Bader-Slusher, Narvon, PA (US); John T. Beneski, JR., Honey Brook, PA (US)

(21) Appl. No.: 14/474,669

(22) Filed: Sep. 2, 2014

Related U.S. Application Data
(63) Continuation of application No. 13/747,705, filed on Jan. 23, 2013, now abandoned.
(60) Provisional application No. 61/589,490, filed on Jan. 23, 2012.

(51) Int. Cl. C12Q 1/68; G01N 33/574

(52) U.S. Cl. CPC C12Q 1/6886; G01N 33/57434; C12Q 2600/158; G01N 2333/904

(57) ABSTRACT

Methods and kits for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; and determining, based on the expression of the one or more biomarkers in the sample that the subject has, or is at risk of having, a proliferative disorder of the prostate gland.

### REFERENCE TO RELATED APPLICATIONS

This application is a continuation of U.S. patent application Ser. No. 13/747,705, filed Jan. 23, 2013, which claims priority from U.S. Provisional Patent Application Ser. No. 61/589,490, filed Jan. 23, 2012, the entire content of both of which is incorporated herein by reference.

### FIELD OF THE INVENTION

The present invention relates generally to methods and compositions for detection and/or assessment of proliferative disorders of the prostate. In specific aspects, the present invention relates to the Class 1 alcohol dehydrogenases, alcohol dehydrogenase 1B and alcohol dehydrogenase 1C, and Class 4 alcohol dehydrogenase, alcohol dehydrogenase 4, and hepatocyte nuclear factor-1B, as biomarkers of the present invention for detection and/or assessment of proliferative disorders of the prostate.

### BACKGROUND OF THE INVENTION

The diagnosis and management of prostate cancer is controversial. Studies comparing surgery versus observation (watchful waiting) demonstrate only a modest reduction in mortality following prostatectomy (Holmburg et al., N Engl J Med., 347(11):781-789, 2002). A measurable benefit of surgery appears to be limited to a poorly defined subpopulation of patients. Widely used biochemical and histopathological criteria such as prostate-specific antigen (PSA), Gleason score, and tumor grade have limited success with respect to prostate cancer stratification (DeMarzo et al., Lancet, 361:955-964, 2003). For patients with a similar clinical profile at the time of diagnosis, known markers cannot predict differences in outcomes (Glinsky et al., J Clin Invest., 113(6):913-923, 2004).

There is a continuing need for methods and compositions to aiding in detection, assessment and treatment of proliferative disorders of the prostate gland in a subject.

### SUMMARY OF THE INVENTION

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; and determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease.

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease; and modifying or initiating an anti-cancer prostate gland treatment for the subject based on determining that the subject has, or is at risk of having, a proliferative disorder of the prostate gland.

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease and treating the subject based on determining that the subject has, or is at risk of having, a proliferative disorder of the prostate gland.

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease; and monitoring the subject for development or progression of a proliferative disorder of the prostate gland based on determining that the subject has, or is at risk of having, a proliferative disorder of the prostate gland.

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease; and monitoring the subject for development or progression of a proliferative disorder of the prostate gland based on determining that the subject has, or is at risk of having, a proliferative disorder of the prostate gland, wherein the monitoring includes assaying a second biological sample comprising prostate gland cells, the second biological sample obtained from the subject at a later time than the biological sample, for expression of one or more biomarkers selected from the group consisting of alcohol dehydrogenase 1B, alcohol dehydrogenase 1C, alcohol dehydrogenase 4 and hepatocyte nuclear factor 1B; and determining, based on the expression of the one or more biomarkers in the sample that the subject has, or is at risk of having, a proliferative disorder of the prostate gland, wherein a decrease in alcohol dehydrogenase 1B expression in the second sample compared to the first sample is indicative of progression of a proliferative disorder of the prostate gland and an increase in alcohol dehydrogenase 1B expression in the second sample compared to the first sample is indicative of amelioration of a proliferative disorder of the prostate gland; wherein a decrease in alcohol dehydrogenase 1C expression in the second sample compared to the first sample is indicative of progression of a proliferative disorder of the prostate gland and an increase in alcohol dehydrogenase 1C expression in the second sample compared to the first sample is indicative of amelioration of a proliferative disorder of the prostate gland; wherein an increase in alcohol dehydrogenase 4 expression in the second sample compared to the first sample is indicative of progression of a proliferative disorder of the prostate gland and a decrease in alcohol dehydrogenase 4 expression in the second sample compared to the first sample is indicative of amelioration of a proliferative disorder of the prostate gland; and wherein an increase in hepatocyte nuclear factor 1B expression in the second sample compared to the first sample is indicative of progression of a proliferative disorder of the prostate gland and a decrease in hepatocyte nuclear factor 1B expression in the second sample compared to the first sample is indicative of amelioration of a proliferative disorder of the prostate gland.

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease; monitoring the subject for development or progression of a proliferative disorder of the prostate gland based on determining that the subject has, or is at risk of having, a proliferative disorder of the prostate gland, wherein the monitoring includes assaying a second biological sample comprising prostate gland cells, the second biological sample obtained from the subject at a later time than the biological sample, for expression of one or more biomarkers selected from the group consisting of: alcohol dehydrogenase 1B, alcohol dehydrogenase 1C, alcohol dehydrogenase 4 and hepatocyte nuclear factor 1B; and determining, based on the expression of the one or more biomarkers in the sample that the subject has, or is at risk of having, a proliferative disorder of the prostate gland, wherein a decrease in alcohol dehydrogenase 1B expression in the second sample compared to the first sample is indicative of progression of a proliferative disorder of the prostate gland and an increase in alcohol dehydrogenase 1B expression in the second sample compared to the first sample is indicative of amelioration of a proliferative disorder of the prostate gland; wherein a decrease in alcohol dehydrogenase 1C expression in the second sample compared to the first sample is indicative of progression of a proliferative disorder of the prostate gland and an increase in alcohol dehydrogenase 1C expression in the second sample compared to the first sample is indicative of amelioration of a proliferative disorder of the prostate gland; wherein an increase in alcohol dehydrogenase 4 expression in the second sample compared to the first sample is indicative of progression of a proliferative disorder of the prostate gland and a decrease in alcohol dehydrogenase 4 expression in the second sample compared to the first sample is indicative of amelioration of a proliferative disorder of the prostate gland; and wherein an increase in hepatocyte nuclear factor 1B expression in the second sample compared to the first sample is indicative of progression of a proliferative disorder of the prostate gland and a decrease in hepatocyte nuclear factor 1B expression in the second sample compared to the first sample is indicative of amelioration of a proliferative disorder of the prostate gland; and selecting a treatment for the subject based on determining that the expression of one or more biomarkers selected from the group consisting of: alcohol dehydrogenase 1B, alcohol dehydrogenase 1C, alcohol dehydrogenase 4 and hepatocyte nuclear factor 1B, is increased or decreased in the second sample compared to the first sample.

Methods for aiding in detection, assessment and treatment of a proliferative disorder of the prostate gland in a subject are provided according to aspects of the present invention which include assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease; monitoring the subject for development or progression of a proliferative disorder of the prostate gland based on determining that the subject has, or is at risk of having, a proliferative disorder of the prostate gland, wherein the monitoring includes assaying a second biological sample comprising prostate gland cells, the second biological sample obtained from the subject at a later time than the biological sample, for expression of one or more biomarkers selected from the group consisting of: alcohol dehydrogenase 1B, alcohol dehydrogenase 1C, alcohol dehydrogenase 4 and hepatocyte nuclear factor 1B; and determining, based on the expression of the one or more biomarkers in the sample that the subject has, or is at risk of having, a proliferative disorder of the prostate gland, wherein a decrease in alcohol dehydrogenase 1B expression in the second sample compared to the first sample is indicative of progression of a proliferative disorder of the prostate gland and an increase in alcohol dehydrogenase 1B expression in the second sample compared to the first sample is indicative of amelioration of a proliferative disorder of the prostate gland; wherein a decrease in alcohol dehydrogenase 1C expression in the second sample compared to the first sample is indicative of progression of a proliferative disorder of the prostate gland and an increase in alcohol dehydrogenase 1C expression in the second sample compared to the first sample is indicative of amelioration of a proliferative disorder of the prostate gland; wherein an increase in alcohol dehydrogenase 4 expression in the second sample compared to the first sample is indicative of progression of a proliferative disorder of the prostate gland and a decrease in alcohol dehydrogenase 4 expression in the second sample compared to the first sample is indicative of amelioration of a proliferative disorder of the prostate gland; and wherein an increase in hepatocyte nuclear factor 1B expression in the second sample compared to the first sample is indicative of progression of a proliferative disorder of the prostate gland and a decrease in hepatocyte nuclear factor 1B expression in the second sample compared to the first sample is indicative of amelioration of a proliferative disorder of the prostate gland; and treating the subject based on determining that the expression of one or more biomarkers selected from the group consisting of: alcohol dehydrogenase 1B, alcohol dehydrogenase 1C, alcohol dehydrogenase 4 and hepatocyte nuclear factor 1B, is increased or decreased in the second sample compared to the first sample.

According to aspects of methods of the present invention, the subject is human.

Methods for aiding in detection, assessment and treatment of a proliferative disorder of the prostate gland in a subject are provided according to aspects of the present invention which include assaying a first biological sample comprising prostate gland cells obtained from the subject to determine the level of expression of one or more of: alcohol dehydrogenase 1B nucleic acid; alcohol dehydrogenase 1C nucleic acid, alcohol dehydrogenase 4 nucleic acid and hepatocyte nuclear factor 1B nucleic acid; particularly mRNA or cDNA; and determining, based on the level of expression of the one or more biomarkers in the sample that the subject has, or is at risk of having, a proliferative disorder of the prostate gland, wherein a decrease in alcohol dehydrogenase 1B nucleic acid expression compared to a standard is indicative of hyperplastic disease or neoplastic disease; wherein a decrease in alcohol dehydrogenase 1C nucleic acid expression compared to a standard is indicative of hyperplastic disease or neoplastic disease; wherein detectable expression of alcohol dehydrogenase 4 nucleic acid compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B nucleic acid expression compared to a standard is indicative of hyperplastic disease or neoplastic disease.

Methods for aiding in detection, assessment and treatment of a proliferative disorder of the prostate gland in a subject are provided according to aspects of the present invention which include assaying a first biological sample comprising prostate gland cells obtained from the subject by a method selected from: quantitative real-time polymerase chain reaction, reverse-transcription polymerase chain reaction, DASL, in-situ hybridization, Northern blot, nuclease protection and microarray analysis, to determine the level of expression of one or more of: alcohol dehydrogenase 1B nucleic acid; alcohol dehydrogenase 1C nucleic acid, alcohol dehydrogenase 4 nucleic acid and hepatocyte nuclear factor 1B nucleic acid; particularly mRNA or cDNA; and determining, based on the level of expression of the one or more biomarkers in the sample that the subject has, or is at risk of having, a proliferative disorder of the prostate gland, wherein a decrease in alcohol dehydrogenase 1B nucleic acid expression compared to a standard is indicative of hyperplastic disease or neoplastic disease; wherein a decrease in alcohol dehydrogenase 1C nucleic acid expression compared to a standard is indicative of hyperplastic disease or neoplastic disease; wherein detectable expression of alcohol dehydrogenase 4 nucleic acid compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B nucleic acid expression compared to a standard is indicative of hyperplastic disease or neoplastic disease.

Methods for aiding in detection, assessment and treatment of a proliferative disorder of the prostate gland in a subject are provided according to aspects of the present invention which include assaying a first biological sample comprising prostate gland cells obtained from the subject by a method selected from: quantitative real-time polymerase chain reaction, reverse-transcription polymerase chain reaction, DASL, in-situ hybridization, Northern blot, nuclease protection and microarray analysis, to determine the level of expression of one or more of: alcohol dehydrogenase 1B nucleic acid; alcohol dehydrogenase 1C nucleic acid, alcohol dehydrogenase 4 nucleic acid and hepatocyte nuclear factor 1B nucleic acid; particularly mRNA or cDNA; determining, based on the level of expression of the one or more biomarkers in the sample that the subject has, or is at risk of having, a proliferative disorder of the prostate gland, wherein a decrease in alcohol dehydrogenase 1B nucleic acid expression compared to a standard is indicative of hyperplastic disease or neoplastic disease; wherein a decrease in alcohol dehydrogenase 1C nucleic acid expression compared to a standard is indicative of hyperplastic disease or neoplastic disease; wherein detectable expression of alcohol dehydrogenase 4 nucleic acid compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B nucleic acid expression compared to a standard is indicative of hyperplastic disease or neoplastic disease; and monitoring the subject for development or progression of a proliferative disorder of the prostate gland based on determining that the subject has, or is at risk of having, a proliferative disorder of the prostate gland, wherein the monitoring includes assaying a second biological sample comprising prostate gland cells by a method selected from: quantitative real-time polymerase chain reaction, reverse-transcription polymerase chain reaction, DASL, in-situ hybridization. Northern blot, nuclease protection and microarray analysis, to determine the level of expression of one or more of: alcohol dehydrogenase 1B nucleic acid; alcohol dehydrogenase 1C nucleic acid, alcohol dehydrogenase 4 nucleic acid and hepatocyte nuclear factor 1B nucleic acid; particularly mRNA or cDNA, in the second biological sample obtained from the subject at a later time than the first biological sample, and determining, based on the expression of one or more of: alcohol dehydrogenase 1B nucleic acid; alcohol dehydrogenase 1C nucleic acid, alcohol dehydrogenase 4 nucleic acid and hepatocyte nuclear factor 1B nucleic acid in the sample that the subject has, or is at risk of having, a proliferative disorder of the prostate gland, wherein a decrease in alcohol dehydrogenase 1B nucleic acid expression in the second sample compared to the first sample is indicative of progression of a proliferative disorder of the prostate gland and an increase in alcohol dehydrogenase 1B nucleic acid expression in the second sample compared to the first sample is indicative of amelioration of a proliferative disorder of the prostate gland; wherein a decrease in alcohol dehydrogenase 1C nucleic acid expression in the second sample compared to the first sample is indicative of progression of a proliferative disorder of the prostate gland and an increase in alcohol dehydrogenase 1C nucleic acid expression in the second sample compared to the first sample is indicative of amelioration of a proliferative disorder of the prostate gland; wherein an increase in alcohol dehydrogenase 4 nucleic acid expression in the second sample compared to the first sample is indicative of progression of a proliferative disorder of the prostate gland and a decrease in alcohol dehydrogenase 4 nucleic acid expression in the second sample compared to the first sample is indicative of amelioration of a proliferative disorder of the prostate gland; and wherein an increase in hepatocyte nuclear factor 1B nucleic acid expression in the second sample compared to the first sample is indicative of progression of a proliferative disorder of the prostate gland and a decrease in hepatocyte nuclear factor 1B nucleic acid expression in the second sample compared to the first sample is indicative of amelioration of a proliferative disorder of the prostate gland.

A sample obtained from the subject for initial assay or monitoring assay is a sample containing or suspected of containing one or more of: alcohol dehydrogenase 1B protein or nucleic acid, alcohol dehydrogenase 1C protein or nucleic acid, alcohol dehydrogenase 4 protein or nucleic acid and hepatocyte nuclear factor 1B protein or nucleic acid, such as a biopsy sample obtained from prostate or prostate tumor, urine or semen.

According to aspects of methods of the present invention a sample obtained from a subject is assayed by immunoassay to detect one or more of: alcohol dehydrogenase 1B, alcohol dehydrogenase 1C, alcohol dehydrogenase 4 and hepatocyte nuclear factor 1B.

According to aspects of methods of the present invention a sample obtained from a subject is assayed by spectrometry to detect one or more of: alcohol dehydrogenase 1B, alcohol dehydrogenase 1C, alcohol dehydrogenase 4 and hepatocyte nuclear factor 1B.

Kits for aiding in detection, assessment and treatment of a proliferative disorder of the prostate gland in a subject are provided according to aspects of the present invention which include at least one reagent for assay of alcohol dehydrogenase 1B, alcohol dehydrogenase 1C, alcohol dehydrogenase 4, hepatocyte nuclear factor 1B; and at least one reagent for assay of prostate specific antigen or androgen receptor.

Kits for aiding in detection, assessment and treatment of a proliferative disorder of the prostate gland in a subject are provided according to aspects of the present invention which include at least one reagent for assay of alcohol dehydrogenase 1B, at least one reagent for assay of alcohol dehydrogenase 1C, at least one reagent for assay of alcohol dehydrogenase 4 and at least one reagent for assay of hepatocyte nuclear factor 1B; and at least one reagent for assay of prostate specific antigen or androgen receptor.

Kits for aiding in detection, assessment and treatment of a proliferative disorder of the prostate gland in a subject are provided according to aspects of the present invention which include at least one probe and/or at least one primer for assay of alcohol dehydrogenase 1B mRNA or cDNA, alcohol dehydrogenase 1C mRNA or cDNA, alcohol dehydrogenase 4 mRNA or cDNA, hepatocyte nuclear factor 1B mRNA or cDNA; and at least one reagent for assay of prostate specific antigen or androgen receptor.

Kits for aiding in detection, assessment and treatment of a proliferative disorder of the prostate gland in a subject are provided according to aspects of the present invention which include at least one probe and/or at least one primer for assay of alcohol dehydrogenase 1B mRNA or cDNA, alcohol dehydrogenase 1C mRNA or cDNA, alcohol dehydrogenase 4 mRNA or cDNA, hepatocyte nuclear factor 1B mRNA or cDNA; and at least one reagent for assay of prostate specific antigen mRNA or cDNA or androgen receptor mRNA or cDNA.

Kits for aiding in detection, assessment and treatment of a proliferative disorder of the prostate gland in a subject are provided according to aspects of the present invention which include at least one reagent for assay of alcohol dehydrogenase 4; and at least one reagent for assay of an Ancestry Informative marker indicative of African descent.

Kits for aiding in detection, assessment and treatment of a proliferative disorder of the prostate gland in a subject are provided according to aspects of the present invention which include at least one probe and/or at least one primer for assay of alcohol dehydrogenase 4 mRNA or cDNA; and at least one probe and/or at least one primer for assay of an Ancestry Informative marker indicative of African descent.

Kits for aiding in detection, assessment and treatment of a proliferative disorder of the prostate gland in a subject are provided according to aspects of the present invention which include at least one probe and/or at least one primer for assay of alcohol dehydrogenase 4 mRNA or cDNA; and a questionnaire form for obtaining the subject's racial self-identification information to determine if the subject self-identifies as of African descent or not.

### BRIEF DESCRIPTION OF THE DRAWINGS

FIG. 1A shows an image of hematoxylin & eosin (H&E) staining of normal prostate tissue;

FIG. 1B shows an image of results of immunocytochemistry using an antibody which recognizes both alcohol dehydrogenase 1 and 4 (ADH 1/4) on normal prostate tissue;

FIG. 1C shows an image of density analysis of immunocytochemistry shown in FIG. 1B;

FIG. 1D shows an image of H&E staining of benign prostatic hyperplasia (BPH) tissue;

FIG. 1E shows an image of results of immunocytochemistry using an ADH 1/4 antibody on benign prostatic hyperplasia tissue;

FIG. 1F shows an image of density analysis of immunocytochemistry shown in FIG. 1E;

FIG. 1G shows an image of H&E staining of Gleason pattern 3 tissue;

FIG. 1H shows an image of results of immunocytochemistry using an ADH 1/4 antibody on Gleason pattern 3 tissue;

FIG. 1I shows an image of density analysis of immunocytochemistry shown in FIG. 1H;

FIG. 1J shows an image of H&E staining of Gleason pattern 4 tissue;

FIG. 1K shows an image of results of immunocytochemistry using an ADH 1/4 antibody on Gleason pattern 4 tissue;

FIG. 1L shows an image of density analysis of immunocytochemistry shown in FIG. 1K;

FIG. 1M shows an image of H&E staining of Gleason pattern 5 tissue;

FIG. 1N shows an image of results of immunocytochemistry using an ADH 1/4 antibody on Gleason pattern 5 tissue;

FIG. 1O shows an image of density analysis of immunocytochemistry shown in FIG. 1N;

FIG. 2A shows an image of an H&E stained tissue sample of normal human prostate parenchyma;

FIG. 2B shows an image of density analysis of an ADH 1/4 immunostained tissue sample of normal human prostate parenchyma;

FIG. 2C shows an image of density analysis of an ADH 1/4 immunostained tissue sample of benign prostatic hyperplasia tissue;

FIG. 2D shows an image of density analysis of an ADH 1/4 immunostained tissue sample of Gleason pattern 3 tissue;

FIG. 3A shows an image of Gleason pattern 3 tissue stained with H&E;

FIG. 3B shows an image of a no-antibody control incubated with PBS instead of ADH antibody;

FIG. 3C shows an image of prostate tissue immunostained with an ADH 1/4 antibody;

FIG. 3D shows an image of prostate tissue immunostained with an androgen receptor (AR) antibody;

FIG. 3E shows an image of prostate tissue immunostained with an prostate specific antigen (PSA) antibody;

FIG. 3F shows an image of prostate tissue immunostained with a cytokeratin-18 (CK18) antibody;

FIG. 4A shows an image of BPH tissue immunostained with an antibody specific to 34 Beta E12 and counterstained with hematoxylin QS;

FIG. 4B shows an image of BPI-1 tissue immunostained with an antibody specific to ADH1B;

FIG. 4C shows an image of BPH tissue immunostained with an antibody specific to 34 Beta E12 and counterstained with hematoxylin QS;

FIG. 4D shows an image of BPH tissue immunostained with an antibody specific to ADH1B;

FIG. 4E shows an image of BPH tissue double immunostained with ADH1B and 34 Beta E12 antibodies;

FIG. 5 is a graph showing expression of epithelial differentiation markers AR, PSA and CK18 in BPH and prostatic adenocarcinoma (PCa) samples (+SEM);

FIG. 6 is a graph showing validation of RPLPO endogenous control used for qRT-PCR analysis of FFPE samples;

FIG. 7 is a graph showing expression of ADH1B in normal, BPH and PCa formalin-fixed paraffin embedded tissue (FFPE) (±SEM);

FIG. 8A is a graph of qRT-PCR data showing expression of ADH1B and hepatocyte nuclear factor 1B (HNF1B) in FFPE tissue samples with BPH alone;

FIG. 8B is a graph of qRT-PCR data showing expression of ADH1B and HNF1B in FFPE tissue samples with BPH, BPH with chronic inflammation and/or prostatitis;

FIG. 8C is a graph of qRT-PCR data showing expression ADH1B and HNF1B expression in FFPE samples with BPH, BPH with chronic inflammation, prostatitis or metaplasia, or malignant PCa;

FIG. 9A is a graph showing relative ADH1B expression in 9 BPH samples as measured by qRT-PCR;

FIG. 9B is a graph showing relative HNF1B expression in corresponding 9 BPH samples as measured by qRT-PCR;

FIG. 9C is a graph showing relative AR expression in corresponding 9 BPH samples as measured by qRT-PCR;

FIG. 9D is a graph showing relative PSA expression in corresponding 9 BPH samples as measured by qRT-PCR; and

FIG. 10 is a graph showing the inferred proportion of African Ancestry in 21 individuals resulting from the analysis of 92 AIM markers (SNPs).

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, Pa., 2003; Herdewijn, P. (Ed.), Oligonucleotide Synthesis: Methods and Applications, Methods in Molecular Biology, Humana Press, 2004; A. Nagy, M. Gertsenstein, K. Vintersten, R. Behringer, Manipulating the Mouse Embryo: A Laboratory Manual, 3rd edition, Cold Spring Harbor Laboratory Press; Dec. 15, 2002, ISBN-10: 0879695919; Kursad Turksen (Ed.), Embryonic stem cells: methods and protocols in Methods Mol Biol. 2002; 185, Humana Press; Current Protocols in Stem Cell Biology, ISBN: 9780470151808.

The singular terms “a,” “an,” and “the” are not intended to be limiting and include plural referents unless explicitly state or the context clearly indicates otherwise.

The human alcohol dehydrogenases (ADH) exist in multiple molecular forms that are grouped into five classes (Edenberg H., Prog Nucleic Acid Res Mol Biol. 2000; 64:295-341). Members of class 1 ADH and class 4 ADH have been extensively studied for their role in the oxidation of ethanol. Class 1 ADH polypeptides are encoded by 3 specific gene loci, ADH1A, ADH1B, and ADH1C; class 4 ADH is encoded by a single gene, ADH7 (Edenberg H., Prog Nucleic Acid Res Mol Biol. 2000; 64:295-341). The highly homologous ADH1 isozymes function as either homo- or heterodimers of the three gene products whereas ADH4 functions as a homodimer. The ADH1/ADH4 isozymes have different spatial and temporal expression patterns as well as substrate affinities (Edenberg H., Prog Nucleic Acid Res Mol Biol. 2000; 64:295-341; Pares X, et al., Cell Mol Life Sci. 2008; 65:3936-3949). Polymorphisms in ADH1B. ADH1C, and ADH4 result in functional differences in the kinetic properties of the isozymes and have been associated with cancer risk, alcoholism, and substance dependence (Visvanathan K, et al., Alcohol Clin Exp Res. 2007; 31:467-476; Luo X, et al., Pharmacogenet Genomics. 2005; 15:755-768).

The term “alcohol dehydrogenase” generally refers to a family of enzymes which includes various isoforms including, but not limited to, class 1 alcohol dehydrogenase (abbreviated ADH1) and class 4 alcohol dehydrogenase (abbreviated ADH4). The term “class 1 alcohol dehydrogenase” refers to a family of enzymes which includes various isoforms including, but not limited to, alcohol dehydrogenase 1A (abbreviated ADH1A) alcohol dehydrogenase 1B (abbreviated ADH1B) and alcohol dehydrogenase 1C (abbreviated ADH1C). The full names and abbreviations of alcohol dehydrogenases mentioned herein are used interchangeably throughout.

Alcohol dehydrogenase isoforms are well-known in the art. Nucleic acid and amino acid sequences for ADH1B are described in Tanaka, F. et al., Gut 59 (11), 1457-1464, 2010; Yang, S. J. et al., World J. Gastroenterol. 16 (33), 4210-4220, 2010; Weng, H. et al., Mutat. Res. 701 (2), 132-136, 2010; Zhang, G. et al., PLoS ONE 5 (10), E13679 2010; Husemoen, L. L. et al., PLoS ONE 5 (8), E11735, 2010; Hurley, T. D. et al., Proc. Natl. Acad. Sci. U.S.A. 88 (18), 8149-8153, 1991; Winter, L. A. et al., Gene 91 (2), 233-240 1990; Stewart, M. J. et al., Gene 90 (2), 271-279, 1990; Carr, L. G. and Edenberg, H. J., J. Biol. Chem. 265 (3), 1658-1664, 1990; and Lange, L. G. et al., Biochemistry 15 (21), 4687-4693 (1976).

Nucleic acid and amino acid sequences for ADH1C are described in Boyles, A. L. et al., Am. J. Epidemiol. 172 (8), 924-931, 2010; Bailey, S. D. et al., Diabetes Care 33 (10), 2250-2253, 2010; Levine, A. J. et al., Cancer Epidemiol. Biomarkers Prev. 19 (7), 1812-1821, 2010; Husemoen, L. L. et al., PLoS ONE 5 (8), E11735, 2010; Jugessur, A. et al., PLoS ONE 5 (7), E11493. 2010; Yokoyama, S. et al., Jpn. J. Genet. 67 (2), 167-171, 1992; Hurley, T. D. et al., Gene 90 (2), 271-279, 1990; and Yasunami, M. et al., Biochemistry 15 (21), 4687-4693, 1976.

Nucleic acid and amino acid sequences for ADH4 are described in Flachsbart, F. et al., Mutat. Res. 694 (1-2), 13-19, 2010; Wei, S. et al., Cancer 116 (12), 2984-2992, 2010; van Beek, J. H. et al., Twin Res Hum Genet 13 (1), 30-42, 2010; Husemoen, L. L. et al., PLoS ONE 5 (8), E11735, 2010; Ross, C. J. et al., Nat. Genet. 41 (12), 1345-1349, 2009; Zgombic-Knight, M. et al., J. Biol. Chem. 270 (9), 4305-4311, 1995; Kedishvili, N. Y., et al., J. Biol. Chem. 270 (8), 3625-3630, 1995; Cheung, B. et al., Alcohol. Clin. Exp. Res. 19 (1), 185-186, 1995; Farres, J. et al., Eur. J. Biochem. 224 (2), 549-557, 1994; and Pares, X. et al., FEBS Lett. 303 (1), 69-72, 1992.

Hepatocyte nuclear factor-1B (HNF1B) is a highly conserved member of the HNF class of homeobox genes that functions during human embryogenesis, playing a major role in organogenesis of the urogenital system as described in Holland et al., BMC Biology, 2007, 5:47-75.

Alcohol dehydrogenase 1B, alcohol dehydrogenase 1C, alcohol dehydrogenase 4 and hepatocyte nuclear factor 1B amino acid and nucleic acid sequences are well-known in the art. Exemplary sequences are included herein for ease of reference and are not intended to be limiting.

ADH1B, ADH1C, ADH4 and HNF1B are collectively referred to herein as biomarkers of the present invention.

Methods for aiding in detection and assessment of a proliferative disorder of the prostate gland in a subject are provided according to aspects of the present invention which include assaying a biological sample obtained from the subject for expression of a biomarker of the present invention and comparison to a standard, wherein detection of a difference in expression of the biomarker in the biological sample compared with the standard is indicative of a proliferative disorder of the prostate gland in the subject.

The term “expression” as used herein refers to production of mRNA as well as to the translation of mRNA into protein.

The term “proliferative disorder” as used herein refers to both neoplastic disease of the prostate, such as prostate cancer, and hyperplastic disease of the prostate, such as benign prostatic hyperplasia.

Methods for aiding in detection and assessment of a proliferative disorder of the prostate gland in a subject are provided according to aspects of the present invention which include assaying a biological sample obtained from the subject for expression of alcohol dehydrogenase 1B, wherein decreased expression of the alcohol dehydrogenase 1B compared to a standard is indicative of a proliferative disorder of the prostate gland in the subject.

It is a finding of the present inventors that alcohol dehydrogenase 1B is expressed in normal prostate epithelium and expression of this protein is decreased in prostate epithelium in hyperplastic disease, such as benign prostatic hyperplasia (BPH), as compared to normal prostate epithelium. It is a finding of the present inventors that alcohol dehydrogenase 1B is decreased in prostate epithelium in neoplastic disease, such as prostate cancer (PCa), as compared to normal prostate epithelium and compared to hyperplastic disease. Prior to the present invention no assay is believed to distinguish between BPH and low grade prostate adenocarcinoma.

Methods for aiding in detection and assessment of a proliferative disorder of the prostate gland in a subject are provided according to aspects of the present invention which include assaying a biological sample obtained from the subject for expression of alcohol dehydrogenase 1B, wherein a decrease in alcohol dehydrogenase 1B expression of about 55% to about 70% compared to a standard is indicative of hyperplastic disease and a decrease in alcohol dehydrogenase 1B of about 90% or greater compared to a standard is indicative of neoplastic disease.

Methods for aiding in detection and assessment of a proliferative disorder of the prostate gland in a subject are provided according to aspects of the present invention which include assaying a biological sample obtained from the subject for expression alcohol dehydrogenase 1C, wherein decreased expression of the alcohol dehydrogenase 1C compared to a standard is indicative of a proliferative disorder of the prostate gland in the subject.

It is a finding of the present inventors that alcohol dehydrogenase 1C is expressed in normal prostate epithelium and expression of this protein is decreased in prostate epithelium in both neoplastic disease, such as prostate cancer, and hyperplastic disease, such as benign prostatic hyperplasia.

Methods for aiding in detection and assessment of a proliferative disorder of the prostate gland in a subject are provided according to aspects of the present invention which include assaying a biological sample obtained from the subject for expression of alcohol dehydrogenase 1C, wherein a decrease in alcohol dehydrogenase 1C expression of about 50%-70% compared to a standard is indicative of hyperplastic disease and a decrease in alcohol dehydrogenase 1C of about 80% or greater compared to a standard is indicative of neoplastic disease.

It is a finding of the present inventors that ADH1B is expressed in normal human prostate tissue. ADH1C is expressed but accounts for approximately 2% of the measured total ADH. The expression of ADH1B is primarily associated with epithelium whereas expression of ADH1C is evenly distributed between epithelium and stroma.

Methods for aiding in detection and assessment of a proliferative disorder of the prostate gland in a subject are provided according to aspects of the present invention which include assaying a biological sample obtained from the subject for expression of HNF1B, wherein an increase in hepatocyte nuclear factor 1B expression of about 50% to about 500% compared to a standard is indicative of hyperplastic disease and an increase in hepatocyte nuclear factor 1B expression of greater than 500% is indicative of neoplastic disease.

The broad use of PSA as a blood marker for the early detection of prostate cancer has created a large population of patients diagnosed with BPI-1 and early-stage prostatic adenocarcinoma (PCa). The heterogeneity in outcomes despite the common clinical profile of these patients represents a highly significant health care challenge. It is a finding of the present inventors that combined ADH1B/HNF1B expression profile can accurately discriminate between these subgroups of early prostate cancer patients and predict differing outcomes. Methods of the present invention including assay of ADH1B and HNF1B distinguish early stage PCa from BPH and identify forms of BPH that will progress to adenocarcinoma from the indolent forms of prostatic disease.

Methods for aiding in detection and assessment of a proliferative disorder of the prostate gland in a subject are provided according to aspects of the present invention which include assaying a biological sample obtained from the subject for expression of ADH1B and HNF1B, wherein an increase in hepatocyte nuclear factor 1B expression of about 50% to about 500% compared to a standard is indicative of hyperplastic disease and an increase in hepatocyte nuclear factor 1B expression of greater than 500% is indicative of neoplastic disease.

Methods for aiding in detection and assessment of a proliferative disorder of the prostate gland in a subject are provided according to aspects of the present invention which include assaying a biological sample obtained from the subject for expression of ADH1B and HNF1B, wherein a decrease in alcohol dehydrogenase 1B expression of about 55% to about 70% compared to a standard is indicative of hyperplastic disease and a decrease in alcohol dehydrogenase 1B of about 90% or greater compared to a standard is indicative of neoplastic disease and wherein an increase in hepatocyte nuclear factor 1B expression of about 50% to about 500% compared to a standard is indicative of hyperplastic disease and an increase in hepatocyte nuclear factor 1B expression of greater than 500% is indicative of neoplastic disease.

It is a finding of the present inventors that expression of ADH4 is detectable in men of African descent with prostate cancer and/or prostatic disease. ADH4 are not detectable in men of African descent with normal prostate epithelium or in the prostate epithelium of Caucasian men with or without prostatic disease.

Methods for aiding in detection and assessment of a proliferative disorder of the prostate gland in a subject of African descent are provided according to aspects of the present invention which include assaying a biological sample obtained from the subject for expression of ADH4, wherein increased expression of ADH4 compared to a standard is indicative of a proliferative disorder of the prostate gland of a subject of African descent.

Methods for aiding in detection and assessment of a proliferative disorder of the prostate gland in a subject are provided according to aspects of the present invention which include assaying a biological sample obtained from the subject for expression of ADH4, wherein detection of expression of the ADH4 is indicative of a proliferative disorder of the prostate gland of a subject of African descent.

Methods for identifying a subject having an increased risk for the development of prostatic adenocarcinoma are provided according to aspects of the present invention which include assaying a biological sample obtained from the subject for expression of ADH4, wherein detection of expression of the ADH4 compared to a standard is indicative of an increased risk for the development of prostatic adenocarcinoma in the subject.

The term “of African descent” and “African-American” are used interchangeably herein to refer to individuals who self-identify as being of African descent, individuals who self-identify as being of African-American, and individuals determined to have genetic markers correlated with African ancestry, also called Ancestry Informative Markers (AIM), see Tables III and IV.

It is a finding of the present inventors that expression of ADH4 is detectable in African-American men with prostate cancer and/or prostatic disease. ADH4 are not detectable in African-American men with normal prostate epithelium or in the prostate epithelium of Caucasian men with or without prostatic disease.

Aspects of methods of the present invention include assay of one or more AIM to identify a subject as of African descent and/or African-American. AIM are described herein and in Yaeger, et al., Cancer Epidemiological Biomarkers 17(6), pgs. 1329-1338, 2008 and Pritchard J K, et al., Genetics, 2000; 155:945-959.

According to aspects of the present invention, assays for one or more biomarkers of the present invention are used to monitor prostate health and/or disease in a subject. Thus, for example, a test sample is obtained from the subject before treatment of a proliferative disorder of the prostate and at one or more times during and/or following treatment in order to assess effectiveness of the treatment. In a further example, a test sample is obtained from the subject at various times in order to assess prostate health or the course or progress of a proliferative disorder of the prostate or healing.

Assays to detect expression of a biomarker of the present invention in a sample obtained from a subject is accomplished using any of various well-known techniques, such as nucleic acid amplification methods, particularly for detection of mRNA expression and assessment of levels of mRNA expression, including, but not limited to, polymerase chain reaction (PCR); in-situ hybridization; Northern blot analysis; nuclease protection assay; RNase protection assay; microarray analysis; and cDNA-mediated Annealing, Selection, extension, and Ligation (DASL) assay (Chow et al., Front Genet. 2012, 3:11).

Nucleic acid template, such as mRNA or cDNA, for use in a method described herein is obtained by any of various techniques well-known in the art, exemplified by techniques described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; and Maliga, P., Methods in Plant Molecular Biology, Cold Spring Harbor Laboratory Press, New York, 1995.

The term “polymerase chain reaction” (PCR) encompasses various well-known methods of detecting expression of a nucleic acid including, but not limited to, standard polymerase chain reaction, ligase chain reaction, phi-29 PCR, reverse transcription-polymerase chain reaction (RT-PCR) such as quantitative and semi-quantitative RT-PCR, and real-time quantitative RT-PCR. PCR and related methods are well-known molecular biology techniques, disclosed, for example, in U.S. Pat. Nos. 4,683,195 4,683,202, and 4,965,188; and in Bieche et al. Novel approach to quantitative polymerase chain reaction using real-time detection: application to the detection of gene amplification in breast cancer. International Journal of Cancer, 78:661-666, 1998; Berndt, C. et al., Quantitative polymerase chain reaction using a DNA hybridization assay based on surface-activated microplates, Analytical Biochemistry, 225:252-257, 1995; Gibson, U. et al., A Novel Method for Real Time Quantitative RT-PCR, Genome Research, 6:995-1001, 1996; and Piatak et al., Quantitative Competive Polymerase Chain Reaction for Accurate Quantitation of HIV DNA and RNA Species, BioTechniques, 14:70-81, 1993.

The term “primer” refers to a single stranded oligonucleotide, typically about 10-60 nucleotides in length, that may be longer or shorter, and that serves as a point of initiation for template-directed DNA synthesis. Design of oligonucleotide primers suitable for use in amplification reactions is well known in the art, for instance as described in A. Yuryev et al., PCR Primer Design, Humana Press, 2007; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

Appropriate reaction conditions for amplification methods include presence of suitable reaction components including, but not limited to, a polymerase and nucleotide triphosphates. One of skill in the art will be able to determine conditions suitable for amplification of the biomarker nucleic acids with only routine experimentation including choice of factors such as length of an included primer, buffer, nucleotides, pH, Mg salt concentration and temperature. The nucleic acid product of the amplification method optionally contains additional materials such as, but not limited to, non-biomarker nucleic acid sequences, functional groups for chemical reaction and detectable labels, present in the primers and not present in the original DNA template.

Amplification products, such as, but not limited to, PCR products, can be analyzed, such as by gel electrophoresis, fluorescent detection, and/or sequencing to detect a target alcohol dehydrogenase.

A nucleic acid probe is able to specifically hybridize to a target biomarker mRNA or cDNA to detect and/or quantify mRNA or cDNA. A nucleic acid probe can be an oligonucleotide of at least 10, 15, 30, 50 or 100 nucleotides in length, sufficient to specifically hybridize under stringent conditions to a biomarker mRNA or cDNA or complementary sequence thereof.

Methods according to embodiments of the present invention include detection of an ADH1B nucleic acid sequence encoding the ADH amino acid sequence of SEQ ID NO: 37 or an amino acid sequence having at least 95% amino acid sequence identity, at least 96% amino acid sequence identity, at least 97% amino acid sequence identity, at least 98% amino acid sequence identity, at least 99% amino acid sequence identity, or greater amino acid sequence identity with SEQ ID NO: 37. One of skill in the art will recognize that, due to the degeneracy of the genetic code, multiple ADH1B nucleic acid sequences can encode the ADH1B protein of SEQ ID NO: 37 and detection of such alternate sequence is encompassed by methods of the present invention. The ADH1B nucleic acid sequence detected may be an entire ADH1B nucleic acid sequence encoding the ADH1B amino acid sequence of SEQ ID NO:37, such as SEQ ID NO:38, or a portion thereof sufficient to specifically identify the nucleic acid as an ADH1B nucleic acid fragment.

In a non-limiting example, the nucleotide sequence of SEQ ID NO:3 is specific to human ADH1B and probes and primers directed to SEQ ID NO:3, or a fragment thereof having at least 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160 or more nucleotides and having at least 93% identity or homology thereto is used in methods according to aspects of the present invention to assay ADH1B mRNA or cDNA.

Methods according to embodiments of the present invention include detection of an ADH nucleic acid sequence encoding the ADH amino acid sequence of SEQ ID NO:39 or an amino acid sequence having at least 95% amino acid sequence identity, at least 96% amino acid sequence identity, at least 97% amino acid sequence identity, at least 98% amino acid sequence identity, at least 99% amino acid sequence identity, or greater amino acid sequence identity with SEQ ID NO:39. One of skill in the art will recognize that, due to the degeneracy of the genetic code, multiple ADH nucleic acid sequences can encode the ADH1C protein of SEQ ID NO:39 and detection of such alternate sequence is encompassed by methods of the present invention. The ADH1C nucleic acid sequence detected may be an entire ADH1C nucleic acid sequence encoding the ADH1C amino acid sequence of SEQ ID NO:39 such as SEQ ID NO:40, or a portion thereof sufficient to specifically identify the nucleic acid as an ADH nucleic acid fragment.

In a non-limiting example, the nucleotide sequence of SEQ ID NO:4 is specific to human ADH1C and probes and primers directed to SEQ ID NO:4, or a fragment thereof having at least 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380 or more nucleotides and having at least 93% identity or homology thereto is used in methods according to aspects of the present invention to assay ADH1C mRNA or cDNA.

Methods according to embodiments of the present invention include detection of an ADH4 nucleic acid sequence encoding the ADH4 amino acid sequence of SEQ ID NO:41 SEQ ID NO:42 or an amino acid sequence having at least 95% amino acid sequence identity, at least 96% amino acid sequence identity, at least 97% amino acid sequence identity, at least 98% amino acid sequence identity, at least 99% amino acid sequence identity, or greater amino acid sequence identity with SEQ ID NO:41 or SEQ ID NO:42. One of skill in the art will recognize that, due to the degeneracy of the genetic code, multiple ADH4 nucleic acid sequences can encode the ADH4 protein of SEQ ID NO:41 or SEQ ID NO:42 and detection of such alternate sequence is encompassed by methods of the present invention. The ADH4 nucleic acid sequence detected may be an entire ADH4 nucleic acid sequence encoding the ADH4 amino acid sequence of SEQ ID NO:41 such as SEQ ID NO:43, or a portion thereof sufficient to specifically identify the nucleic acid as an ADH4 nucleic acid fragment. Further, the ADH4 nucleic acid sequence detected may be an entire ADH4 nucleic acid sequence encoding the ADH4 amino acid sequence of SEQ ID NO:42 such as SEQ ID NO:44, or a portion thereof sufficient to specifically identify the nucleic acid as an ADH4 nucleic acid fragment.

In a non-limiting example, the nucleotide sequence of SEQ ID NO:5 is specific to human ADH4 and probes and primers directed to SEQ ID NO:5, or a fragment thereof having at least 10, 11, 12, 13, 14, 15, 16, 17, 19, 19, 20, 21, 22, 23, 24, 25, 26, 27, 28, 29, 30, 35, 40, 45, 50, 55, 60, 65 or more nucleotides and having at least 90% identity or homology thereto is used in methods according to aspects of the present invention to assay ADH4 mRNA or cDNA.

Methods according to embodiments of the present invention include detection of an HNF1B nucleic acid sequence encoding the HNF1B amino acid sequence of SEQ ID NO:45 or an amino acid sequence having at least 95% amino acid sequence identity, at least 96% amino acid sequence identity, at least 97% amino acid sequence identity, at least 98% amino acid sequence identity, at least 99% amino acid sequence identity, or greater amino acid sequence identity with SEQ ID NO:45. One of skill in the art will recognize that, due to the degeneracy of the genetic code, multiple HNF1B nucleic acid sequences can encode the HNF1B protein of SEQ ID NO:45 and detection of such alternate sequence is encompassed by methods of the present invention. The HNF1B nucleic acid sequence detected may be an entire HNF1B nucleic acid sequence encoding the HNF1B amino acid sequence of SEQ ID NO:45 such as SEQ ID NO:46, or a portion thereof sufficient to specifically identify the nucleic acid as an HNF1B nucleic acid fragment.

In a non-limiting example, the nucleotide sequence of SEQ ID NO:6 is specific to human HNF1B and probes and primers directed to SEQ ID NO:6, or a fragment thereof having at least 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125 or more nucleotides and having at least 90% identity or homology thereto is used in methods according to aspects of the present invention to assay HNF1B mRNA or cDNA.

In a non-limiting example, the nucleotide sequence of SEQ ID NO:7 is specific to human androgen receptor and probes and primers directed to SEQ ID NO:7, or a fragment thereof having at least 10, 11, 12, 13, 14, 15, 16, 17, 19, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135 or more nucleotides and having at least 90% identity or homology thereto is used in methods according to aspects of the present invention to assay androgen receptor mRNA or cDNA.

In a non-limiting example, the nucleotide sequence of SEQ ID NO:8 is specific to human prostate specific antigen and probes and primers directed to SEQ ID NO:8, or a fragment thereof having at least 10, 11, 12, 13, 14, 15, 16, 17, 19, 19, 20, 21, 22, 23, 24, 25, 26, 27, 28, 29, 30, 35, 40, 45, 50, 55, 60, 65 or more nucleotides and having at least 93% identity or homology thereto is used in methods according to aspects of the present invention to assay prostate specific antigen mRNA or cDNA.

Percent identity refers to the extent of exact correspondence of one amino acid or nucleotide sequence with another. Percent homology refers to the extent of similarity of one amino acid or nucleotide sequence with another. Percent identity and percent homology is determined by comparison of amino acid or nucleic acid sequences, including a reference amino acid or nucleic acid sequence and a putative homologue amino acid or nucleic acid sequence. To determine the percent identity or percent homology of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). The two sequences compared are generally the same length or nearly the same length.

The determination of percent identity or percent homology between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present invention. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules as described in Altschul et al., 1997, Nucleic Acids Res. 25:3389 3402. When utilizing BLAST, Gapped BLAST, and PSI Blast programs, the default parameters of the respective programs (e.g., of XBLAST and NBLAST) are used (see, e.g., the NCBI website). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 is used.

The percent identity or percent homology between two sequences is determined using techniques similar to those described above, with or without allowing gaps. In calculating percent identity, typically only exact matches are counted.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100%, or completely, complementary to a region of the nucleotide sequence 5′-TTAGCTGG-3′. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone.

Assays to detect a biomarker of the present invention in a sample obtained from a subject include any of various techniques, such as binding of a specific binding agent using binding assays. Non-limiting examples of such binding assays include immunoassays and analogous binding assays using aptamers as binding agents.

Particular methods of immunoassay are known in the art and illustratively include enzyme-linked immunosorbent assay (ELISA), immunofluorescence, immunoblot, immunoprecipitation, flow cytometry, immunohistochemistry, immunocytochemistry, and radioimmunoassay. Assay methods may be used to obtain qualitative and/or quantitative results. Specific details of suitable assay methods for both qualitative and quantitative assay of a sample are described in standard references, illustratively including E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; B. K. C. Lo, Antibody Engineering: Methods and Protocols, Methods in Molecular Biology, Humana Press, 2003; F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; Ormerod, M. G., Flow Cytometry: a practical approach, Oxford University Press, 2000; Givan, A. L., Flow Cytometry: first principles, Wiley, New York, 2001; Gorczyca, W., Flow Cytometry in Neoplastic Hematology: morphologic-immunophenotypic correlation, Taylor & Francis, 2006; and J. Sambrook and D. W. Russell, Molecular Cloning: A Laboratory Manual, Cold Spring Harbor Laboratory Press, 3rd Ed., 2001.

Antibodies directed against a biomarker of the present invention can be polyclonal or monoclonal antibodies. Suitable antibodies also include chimeric antibodies, humanized antibodies, and antigen binding antibody fragments and molecules having antigen binding functionality, such as aptamers. Examples of antibody fragments that can be used in aspects of inventive assays include Fab fragments, Fab′ fragments, F(ab′)₂ fragments, Fd fragments, Fv fragments, scFv fragments, and domain antibodies (dAb).

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

Aptamers can be used to assay an alcohol dehydrogenase in a sample obtained from a subject. The term “aptamer” refers to a peptide and/or nucleic acid that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Similarly, peptide-based aptamers are characterized by specific binding to a target wherein the aptamer is not a naturally occurring ligand for the target. Techniques for identification and generation of peptide and nucleic acid aptamers and their use are known in the art as described, for example, in F. M. Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

An assay for one or more biomarkers of the present invention can incorporate a support for attachment of a specific binding agent, such as, but not limited to an antibody or probe. A support with attached specific binding agent can be solid or semi-solid and can be any of various materials such as glass, silicon, paper, a synthetic or naturally occurring polymer, such as polystyrene, polycarbonate, polypropylene, PVDF, nylon, cellulose, agarose, dextran, and polyacrylamide or any other material to which a specific binding agent can be stably attached for use in a binding assay.

A support used can include functional groups for binding to a specific binding agent, such as, but not limited to carboxyl, amine, amino, carboxylate, halide, ester, alcohol, carbamide, aldehyde, chloromethyl, sulfur oxide, nitrogen oxide, epoxy and/or tosyl functional groups. Attachment of a specific binding agent to a support is achieved by any of various methods, illustratively including adsorption and chemical bonding. In one example, 1-Ethyl-3-[3-dimethylaminopropyl]carbodiimide hydrochloride, EDC or EDAC chemistry, can be used to attach a specific binding agent to particles. The specific binding agent can be bonded directly or indirectly to the material of the support, for example, via bonding to a coating or linker disposed on the support. Functional groups, modification thereof and attachment of a binding partner to a support are known in the art, for example as described in Fitch, R. M., Polymer Colloids: A Comprehensive Introduction, Academic Press, 1997.

Such supports can be in any of a variety of forms and shapes including, but not limited to, microtiter plates, microtiter wells, pins, fibers, beads, slides, silicon chips and membranes such as a nitrocellulose or PVDF membrane.

Spectrometric analysis can be used to assay a sample for a biomarker of the present invention. Any of various spectroscopy methods can be used to assay one or more biomarkers of the present invention, including, but not limited to, gas chromatography, liquid chromatography, ion mobility spectrometry, mass spectrometry, liquid chromatography-mass spectrometry (LC-MS or HPLC-MS), ion mobility spectrometry-mass spectrometry, tandem mass spectrometry, gas chromatography-mass spectrometry, matrix-assisted desorption ionization time-of-flight (MALDI-TOF) mass spectrometry, surface-enhanced laser desorption ionization (SELDI) and nuclear magnetic resonance spectroscopy, all of which are well-known to the skill artisan.

For example mass analysis can be used in an assay according to aspects of the present invention. Mass analysis is conducted using, for example, time-of-flight (TOF) mass spectrometry or Fourier transform ion cyclotron resonance mass spectrometry. Mass spectrometry techniques are known in the art and exemplary detailed descriptions of methods for protein and/or peptide assay are found in Li J., et al., Clin Chem., 48(8):1296-304, 2002; Hortin, G. L., Clinical Chemistry 52: 1223-1237, 2006; Hortin, G. L., Clinical Chemistry 52: 1223-1237, 2006; A. L. Burlingame, et al. (Eds.), Mass Spectrometry in Biology and Medicine, Humana Press, 2000; and D. M. Desiderio, Mass Spectrometry of Peptides, CRC Press, 1990.

Any method for detecting function of a biomarker of the present invention in a sample obtained from a subject can be used according to aspects of the present invention.

A sample obtained from a subject is optionally purified for assay according to a method of the present invention. The term “purified” in the context of a sample refers to separation or enrichment of a biomarker relative to at least one other component present in the sample. Sample purification is achieved by techniques illustratively including electrophoretic methods such as gel electrophoresis and 2-D gel electrophoresis; chromatography methods such as HPLC, ion exchange chromatography, affinity chromatography, size exclusion chromatography, thin layer and paper chromatography.

Assay of one or more biomarkers of the present invention in a subject sample is optionally compared to a result of an assay of the corresponding one or more biomarker proteins or nucleic acids of the present invention in a standard.

Standards are well-known in the art and the standard used can be any appropriate standard. In one example, a standard is an amount of one or more biomarkers of the present invention present in a comparable control sample from a control subject. Control samples may be obtained from one or more normal subjects, for example. A standard may be a reference level of one or more biomarkers of the present invention determined in a sample of an individual subject or in a population. A standard may be such a reference level stored in a print or electronic medium for recall and comparison to an assay result.

A standard can be an amount of one or more biomarkers of the present invention present in a comparable sample obtained from the same subject at a different time. For example, a standard can be an amount of one or more biomarkers of the present invention present in a comparable sample obtained from the same subject at an earlier time. A first sample can be obtained from an individual subject at a first time to obtain a subject-specific baseline level of the one or more biomarkers of the present invention in the first sample. A second sample can be obtained from the individual subject at a second time and assayed for one or more biomarkers of the present invention to monitor differences in the levels of the one or more biomarkers of the present invention compared to the first sample, thereby monitoring prostate health and/or disease in the subject. Additional samples can be obtained from the subject at additional time points and assayed for biomarkers to monitor differences in the levels of the corresponding one or more biomarkers of the present invention compared to the first sample, second sample or other samples, thereby monitoring prostate health and/or disease in the subject.

A standard can be an average level of one or more biomarkers of the present invention present in comparable samples of one or more populations. The “average level” is determined by assay of the one or more biomarkers of the present invention in comparable samples obtained from each member of the population. The term “comparable sample” is used to indicate that the samples are of the same type. For example, each of the comparable samples is a urine sample. In a further example, each of the comparable samples is a biopsy sample. In a further example, each of the comparable samples is a semen sample.

A difference detected in levels of one or more biomarkers of the present invention compared to a standard can be an increase or decrease in level of the one or more biomarkers. A difference detected in levels of one or more biomarkers of the present invention compared to a standard can be a detectable level of the one or more biomarkers where the biomarker is undetectable in the standard. A difference detected in levels of one or more biomarkers of the present invention compared to a standard can be an undetectable level of the one or more biomarkers where the biomarker is detectable in the standard.

Assay results can be analyzed using statistical analysis by any of various methods, exemplified by parametric or non-parametric tests, analysis of variance, analysis of covariance, logistic regression for multivariate analysis, Fisher's exact test, the chi-square test, Student's T-test, the Mann-Whitney test, Wilcoxon signed ranks test, McNemar test, Friedman test and Page's L trend test. These and other statistical tests are well-known in the art as detailed in Hicks, C M, Research Methods for Clinical Therapists: Applied Project Design and Analysis, Churchill Livingstone; 5th Ed., 2009; and Freund, R J et al., Statistical Methods, Academic Press; 3rd Ed., 2010.

The term “subject” as used herein refers particularly to human subjects, although compositions and methods of the present invention may be applicable to subjects of other species. The subject can also be a non-human mammal and or a bird, such as, but not limited to, non-human primates, cats, dogs, cows, horses, rabbits, rodents, such as laboratory mice, rats and guinea pigs, pigs, sheep, goats and poultry.

The terms “sample” and “biological sample” are used interchangeably herein to refer to a sample obtained from a subject containing or presumed to contain cells, tissues and fluids of the prostate gland, and more particularly containing or presumed to contain epithelial cells of the prostate gland, such as urine, semen and/or biopsy material obtained from the subject.

The terms “treating” and “treatment” used to refer to treatment of a proliferative disorder of the prostate in a subject include: preventing, inhibiting or ameliorating the cancer in the subject, such as slowing progression of the proliferative disorder of the prostate and/or reducing or ameliorating a sign or symptom of the proliferative disorder of the prostate.

Methods of aiding in diagnosis, assessment and treatment according to aspects of the present invention include initiating or modifying a course of treatment administered or recommended to the subject based on results of one or more assays of one or more biomarkers of the present invention. According to aspects, a course of anti-cancer treatment of the prostate gland is administered or recommended to the subject based on results of one or more assays of one or more biomarkers of the present invention. According to aspects, a more or less rigorous course of anti-cancer treatment of the prostate gland is administered or recommended to the subject based on results of one or more assays of one or more biomarkers of the present invention.

A less rigorous course of anti-cancer treatment includes, but is not limited to, less frequent active surveillance of the subject to detect a proliferative disorder of the prostate and/or worsening or progression of a proliferative disorder of the prostate. Such active surveillance can include: one or more additional assays of one or more of: ADH1B, ADH1C, HNF1B and ADH4; assay of prostate specific antigen, assay of androgen receptor, digital rectal exam, and biopsy.

A more rigorous course of anti-cancer treatment includes, but is not limited to, more frequent or earlier active surveillance of the subject to detect a proliferative disorder of the prostate and/or worsening or progression of a proliferative disorder of the prostate. A more rigorous course of anti-cancer treatment includes, but is not limited to, interstitial prostate brachytherapy, external beam radiotherapy, hormonal therapy, cryotherapy, radical prostatectomy and pharmaceutical therapies such as, but not limited to, administration of an alpha-blocker, illustratively including alfuzosin, doxazosin, tamsulosin, terazosin and silodosin; administration of a 5-alpha-reductase inhibitor, illustratively including dutasteride and finasteride; administration of an anticholinergic agent; or a combination of two or more of an alpha-blocker, a 5-alpha-reductase inhibitor and an anticholinergic agent.

According to aspects, a more rigorous course of anti-cancer treatment of the prostate gland is administered or recommended to the subject based on results of one or more assays of one or more biomarkers of the present invention in which: a decrease in expression of ADH1B compared to a standard is detected, a decrease in expression of ADH1C compared to a standard is detected, an increase in HNF1B is detected, any expression of ADH4 compared to a standard is detected, or a combination of any two or more of these results is detected.

According to aspects, a more rigorous course of anti-cancer treatment of the prostate gland is administered or recommended to a subject of African descent based on results of one or more assays of one or more biomarkers of the present invention in which: a decrease in expression of ADH1B compared to a standard is detected, a decrease in expression of ADH1C compared to a standard is detected, an increase in HNF1B is detected, any expression of ADH4 compared to a standard is detected, or a combination of any two or more of these results is detected.

According to aspects, a less rigorous course of anti-cancer treatment of the prostate gland is administered or recommended to the subject based on results of one or more assays of one or more biomarkers of the present invention in which: an increase in expression of ADH1B compared to a standard is detected, an increase in expression of ADH1C compared to a standard is detected, a decrease in HNF1B is detected compared to a standard, no expression of ADH4 compared to a standard is detected, or a combination of any two or more of these results is detected.

Methods according to aspects of the present invention including assay of one or more biomarkers of the present invention to aid in detection, assessment and treatment of a proliferative disorder of the prostate gland can be performed alone or as part of a panel of biomarkers, such as, but not limited to, biomarkers of disease and/or biomarkers of therapeutic efficacy of various drugs, such as detection of prostate serum antigen (PSA), prostate-specific membrane antigen, androgen receptor and/or cytokeratin.

Methods according to aspects of the present invention include assay of PSA and assay of one or more of: ADH1B, ADH1C, HNF1B and ADH4; in a sample obtained from a subject having, suspected of having or at risk for a proliferative disorder of the prostate to detect the level of PSA and assay of one or more of ADH1B, ADH1C, HNF1B and ADH4, compared to a standard.

According to aspects, a more rigorous course of anti-cancer treatment of the prostate gland is administered or recommended to the subject based on results of one or more assays of one or more biomarkers of the present invention in which: a decrease in expression of ADH1B compared to a standard is detected, a decrease in expression of ADH1C compared to a standard is detected, an increase in HNF1B is detected, any expression of ADH4 compared to a standard is detected; or a combination of any two or more of these results is detected along with an increase in PSA compared to a standard.

According to aspects, a less rigorous course of anti-cancer treatment of the prostate gland is administered or recommended to the subject based on results of one or more assays of one or more biomarkers of the present invention in which: an increase in expression of ADH1B compared to a standard is detected, an increase in expression of ADH 1C compared to a standard is detected, a decrease in HNF1B is detected compared to a standard, no expression of ADH4 compared to a standard is detected; or a combination of any two or more of these results is detected along with a non-elevated level of PSA is detected compared to a standard.

According to aspects, a more rigorous course of anti-cancer treatment of the prostate gland is administered or recommended to the subject based on results of one or more assays of one or more biomarkers of the present invention in which: a decrease in expression of ADH1B compared to a standard is detected, a decrease in expression of ADH1C compared to a standard is detected, an increase in HNF1B is detected, any expression of ADH4 compared to a standard is detected; or a combination of any two or more of these results is detected along with detection of high grade prostate cancer by microscopic analysis of tumor biopsy material, such as identification of a prostatic adenocarcinoma Gleason score 6-10 tumor.

According to aspects, a less rigorous course of anti-cancer treatment of the prostate gland is administered or recommended to the subject based on results of one or more assays of one or more biomarkers of the present invention in which: an increase in expression of ADH1B compared to a standard is detected, an increase in expression of ADH1C compared to a standard is detected, a decrease in HNF1B is detected compared to a standard, no expression of ADH4 compared to a standard is detected; or a combination of any two or more of these results is detected along with detection of a benign growth or low grade tumor by microscopic analysis of tumor biopsy material, such as identification of a benign growth of the prostate gland or prostatic adenocarcinoma Gleason grade 1-3 tumor.

According to aspects, a more rigorous course of anti-cancer treatment of the prostate gland is administered or recommended to the subject based on results of one or more assays of one or more biomarkers of the present invention in which: a decrease in expression of ADH1B compared to a standard is detected, a decrease in expression of ADH compared to a standard is detected, an increase in HNF1B is detected, any expression of ADH4 compared to a standard is detected; or a combination of any two or more of these results is detected along with detection of prostate abnormality by digital rectal exam.

According to aspects, a less rigorous course of anti-cancer treatment of the prostate gland is administered or recommended to the subject based on results of one or more assays of one or more biomarkers of the present invention in which: an increase in expression of ADH1B compared to a standard is detected, an increase in expression of ADH compared to a standard is detected, a decrease in HNF1B is detected compared to a standard, no expression of ADH4 compared to a standard is detected; or a combination of any two or more of these results is detected along with detection of normal prostate by digital rectal exam.

Kits for aiding in detection, assessment and treatment of a proliferative disorder of the prostate gland in a subject are provided according to aspects of the present invention which include at least one reagent for detection of a biomarker of the present invention, such as alcohol dehydrogenase 1B, alcohol dehydrogenase 1C, alcohol dehydrogenase 4 and/or hepatic nuclear factor 1B.

Kits according to aspects of the present invention optionally include one or more components for use in an assay of the present invention such as a primer, probe, antibody or aptamer which recognizes a biomarker of the present invention, a liquid such as a buffer and/or solution used in an assay, a container, a detectable label for labeling a primer, probe, antibody or aptamer directly or indirectly, a standard, a negative control and a positive control, and optionally instructions for use.

Kits for aiding in detection, assessment and treatment of a proliferative disorder of the prostate gland in a subject are provided according to aspects of the present invention which include at least one reagent for assay of alcohol dehydrogenase 1B, alcohol dehydrogenase 1C, alcohol dehydrogenase 4, hepatocyte nuclear factor 1B; and at least one reagent for assay of prostate specific antigen or androgen receptor.

Kits for aiding in detection, assessment and treatment of a proliferative disorder of the prostate gland in a subject are provided according to aspects of the present invention which include at least one reagent for assay of alcohol dehydrogenase 1B, at least one reagent for assay of alcohol dehydrogenase 1C, at least one reagent for assay of alcohol dehydrogenase 4 and at least one reagent for assay of hepatocyte nuclear factor 1B; and at least one reagent for assay of prostate specific antigen or androgen receptor.

Kits for aiding in detection, assessment and treatment of a proliferative disorder of the prostate gland in a subject are provided according to aspects of the present invention which include at least one probe and/or at least one primer and/or at least two primers for assay of alcohol dehydrogenase 1B mRNA or cDNA, alcohol dehydrogenase 1C mRNA or cDNA, alcohol dehydrogenase 4 mRNA or cDNA, hepatocyte nuclear factor 1B mRNA or cDNA; and at least one reagent for assay of prostate specific antigen and/or androgen receptor protein, mRNA or cDNA.

Kits for aiding in detection, assessment and treatment of a proliferative disorder of the prostate gland in a subject are provided according to aspects of the present invention which include at least one probe and/or at least one primer and/or at least two primers for assay of alcohol dehydrogenase 1B mRNA or cDNA, alcohol dehydrogenase 1C mRNA or cDNA, alcohol dehydrogenase 4 mRNA or cDNA, hepatocyte nuclear factor 1B mRNA or cDNA; and at least one probe and/or at least one primer for assay of prostate specific antigen mRNA or cDNA or androgen receptor mRNA or cDNA.

Kits for aiding in detection, assessment and treatment of a proliferative disorder of the prostate gland in a subject are provided according to aspects of the present invention which include at least one probe and/or at least one primer and/or at least two primers for each of alcohol dehydrogenase 1B mRNA or cDNA, alcohol dehydrogenase 1C mRNA or cDNA, alcohol dehydrogenase 4 mRNA or cDNA, hepatocyte nuclear factor 1B mRNA or cDNA; and at least one probe and/or at least one primer for assay of prostate specific antigen mRNA or cDNA or androgen receptor mRNA or cDNA.

Kits for aiding in detection, assessment and treatment of a proliferative disorder of the prostate gland in a subject are provided according to aspects of the present invention which include at least one reagent for assay of alcohol dehydrogenase 4; and at least one reagent for assay of an Ancestry Informative marker indicative of African descent.

Kits for aiding in detection, assessment and treatment of a proliferative disorder of the prostate gland in a subject are provided according to aspects of the present invention which include at least one probe and/or at least one primer and/or at least two primers for assay of alcohol dehydrogenase 4 mRNA or cDNA; and at least one probe and/or at least one primer for assay of an Ancestry Informative marker indicative of African descent.

Kits for aiding in detection, assessment and treatment of a proliferative disorder of the prostate gland in a subject are provided according to aspects of the present invention which include at least one probe and/or at least one primer and/or at least two primers for assay of alcohol dehydrogenase 4 mRNA or cDNA; and a questionnaire form for obtaining the subject's racial self-identification information to determine if the subject self-identifies as of African descent or not.

Embodiments of inventive compositions and methods are illustrated in the following examples. These examples are provided for illustrative purposes and are not considered limitations on the scope of inventive compositions and methods.

### EXAMPLES

### Formalin Fixed, Paraffin Embedded (FFPE) Tissue

Sixty-four archived samples of prostate tissue were obtained as formalin-fixed, paraffin embedded tissue blocks (FFPE) from routine histological files. The age, race, and diagnosis were transmitted with each patient sample. Thirty-four samples were from African-American patients (B) ranging in age from 48 to 90 years and 30 samples were from Caucasian patients (W) ranging in age from 50 to 82 years. Thirty-one samples were from patients diagnosed with BPH, 32 samples were from patients diagnosed with adenocarcinomas of varying Gleason scores and one patient was diagnosed with inflammation of the prostate. Table I summarizes the information associated with each sample.

TABLE I
FFPE Sample population description.

| Individual | Age | Race | Diagnosis |
|---|---|---|---|
| 11551 | 61 | B | BPH, I |
| 750 | 57 | B | BPH, I |
| 7853 | 75 | B | BPH |
| 1819 | 75 | B | BPH |
| 931 | 86 | B | BPH |
| 4440 | 77 | B | BPH |
| 1152 | 72 | B | BPH |
| 3450 | 77 | B | BPH |
| 3297 | 62 | B | BPH |
| 1213 | 75 | B | BPH |
| 1207 | 81 | B | BPH |
| 4641 | 71 | B | BPH |
| 3233 | 69 | B | BPH |
| 911 | 70 | B | BPH |
| 3587 | 77 | B | BPH |
| 8158 | 62 | B | BPH |
| 6131 | 71 | B | BPH, I |
| 4382 | 64 | B | BPH, I |
| 3897 | 58 | B | BPH, I, M |
| 8188 | 68 | B | BPH, M |
| 8821 | 81 | B | BPH, M |
| 3696 | 59 | B | BPH, P |
| 2910 | 53 | B | I |
| 1968 | 55 | B | PCa Gleason Grade 6, HPIN, BPH |
| 3804 | 52 | B | PCa Gleason Grade 7 |
| 6635 | 60 | B | PCa Gleason Grade 7, HPIN, BPH |
| 7577 | 48 | B | PCa Gleason Grade 7 |
| 2394 | 56 | B | PCa Gleason Grade 7 |
| 2443 | 50 | B | PCa Gleason Grade 7, HPIN, BPH, I |
| 3738 | 57 | B | PCa Gleason Grade 7 |
| 2669 | 57 | B | PCa Gleason Grade 7 |
| 5964 | 90 | B | PCa Gleason Grade 9 |
| 7291 | 58 | B | PCa Gleason Grade 9, HPIN |
| 2595 | 50 | B | PCa Gleason Grade 9 |
| 144 | 82 | W | BPH, I |
| 2123 | 54 | W | BPH |
| 11985 | 59 | W | BPH |
| 1798 | 81 | W | BPH |
| 3800 | 69 | W | BPH |
| 3202 | 63 | W | BPH |
| 1002 | 66 | W | BPH, I |
| 101 | 63 | W | BPH, I |
| 1778 | 74 | W | BPH, I |
| 2350 | 59 | W | Pca Gleason Grade 6, HPIN, BPH, I, M |
| 2372 | 58 | W | PCa Gleason Grade 6 |
| 2876 | 65 | W | PCa Gleason Grade 6, BPH |
| 4344 | 61 | W | PCa Gleason Grade 6 |
| 2031 | 64 | W | PCa Gleason Grade 6, BPH |
| 2908 | 68 | W | PCa Gleason Grade 6, I |
| 929 | 63 | W | PCa Gleason Grade 6, HPIN |
| 15245 | 60 | W | PCa Gleason Grade 6, HPIN |
| 1044 | 50 | W | PCa Gleason Grade 6, HPIN |
| 201 | 58 | W | PCa Gleason Grade 7, HPIN |
| 596 | 64 | W | PCa Gleason Grade 7, BPH, P |
| 2620 | 66 | W | PCa Gleason Grade 7, M, I |
| 3352 | 67 | W | PCa Gleason Grade 7, HPIN, BPH, P |
| 1657 | 67 | W | PCa Gleason Grade 7 |
| 360 | 74 | W | PCa Gleason Grade 7, BPH, HPIN, I |
| 7962 | 62 | W | PCa Gleason Grade 7, HPIN |
| 4203 | 64 | W | PCa Gleason Grade 7, HPIN, BPH |
| 1983 | 68 | W | PCa Gleason Grade 8, HPIN |
| 14116 | 73 | W | PCa Gleason Grade 8, HPIN, BPH, I |
| 1131 | 68 | W | PCa Gleason Grade 8, HPIN, I |
| 3475 | 57 | W | PCa Gleason Grade 9 |

Key:
Pca = Prostatic Adenocarcinoma,
BPH = Benign Prostatic Hyperplasia,
I = Chronic inflammation,
HPIN = High Grade Prostatic Intraepithelial Neoplasia,
M = Metaplasia,
P = Prostatitis

Two additional FFPE samples of normal prostate tissue were purchased (Cybrdi, Rockville, Md.). One sample was from a 46-year-old African-American male (PN_{AA}) and another was from a 77-year-old Caucasian male (PN_{cau}). Neither of these patients had any history of prostatic disease or cancer.

Urine Samples from Young Men with No History of Prostate Disease

Urine samples were collected from 170 healthy male participants with no history of prostatic disease between the ages of 18 and 45 years of age. Each participant was asked to complete a survey to self-identify their ethnicity and to provide personal and family histories of prostate disease including benign prostatic hyperplasia (BPH), chronic inflammation (CI), prostatic intraepithelial neoplasia (PIN) and prostatic adenocarcinoma (PCa).

Immunocytochemistry

All samples were sectioned at 8 μm. Sections were placed on UltraStick slides (Gold Seal, Portsmouth, N.H.) and deparaffinized using two incubations in xylene, followed by two incubations each in decreasing series of ethanol at 100%, 95% and 70%. Samples were then incubated in two changes of PBS. Endogenous alkaline phosphatase was inhibited using 0.2 N HCl for 5 minutes at room temperature. Slides where then washed in at least 3 changes of PBS for a total of 10 minutes. Nonspecific interactions were blocked by incubation with normal serum in PBS. The distribution of antibody targets in prostate tissue was analyzed using two different alcohol dehydrogenase antibodies: 1) a IgG1 mouse monoclonal antibody from clone 1E5 raised against alcohol dehydrogenase purified from human liver and 2) a Protein A purified polyclonal rabbit ADH1B antibody from clone E-24 raised against synthetic ADH1B peptide of human origin. The distribution of Prostate Specific Antigen (PSA) was analyzed using an IgG1 mouse monoclonal antibody from clone 5A11E9 raised against a purified recombinant fragment of PSA (aa26-251) expressed in E. Coli. The distribution of Cytokeratin 18 (CK18) was determined using an IgG1 mouse monoclonal antibody from clone DC 10 produced by immunizing mice with human PMC-42 breast carcinoma cells. The distribution of androgen receptor (AR) was determined using an IgG1 mouse monoclonal antibody raised against the synthetic peptide: KSTEDTAEYSPFKGGYT (SEQ ID NO:1), corresponding to amino acids 299-315 of human androgen receptor. The distribution of pan-Cytokeratin (34betaE12) was determined using a mouse IgG1 mouse monoclonal antibody from clone sc-58823 produced by immunizing mice with solubilized pan-Cytokeratin of human stratum corneum origin.

Each antibody except 34betaE12 was used in immunohistochemistry of 23 biopsy samples from Main Line Health Systems (Paoli, Pa.), and 40 biopsy samples from Karmanos Cancer Institute (Detroit, Mich.). 34betaE12 was used in immunohistochemistry of 10 FFPE samples from Main Line Health and the purchased sample PN_{cau}, 3 samples were BPH samples and 7 were PCa samples. For all samples, serial sections were cut at 8 μm and mounted on UltraStick slides (Thermo-Fisher Scientific). Each antibody was diluted (monoclonal ADH 1:100, polyclonal ADH1B 1:100, PSA 1:500, CK18 1:50, AR 1:100, 34betaE12 1:100) and applied to the slides in an overnight incubation. A biotinylated anti-mouse secondary antibody (1:4,000 dilution) or a biotinylated anti-rabbit antibody (1:4,000 dilution) was used for detection of all primary antibodies followed by incubation with an avidin:biotinylated enzyme complex and Vector Red as a chromogenic substrate for all antibodies except 34betaE12 which used peroxidase. (Vector Laboratories, Burlingame, Calif.). Samples were examined and documented on an Olympus DP21 2 megapixel color camera affixed to an Olympus BX60 microscope (Lake Success, N.Y.) and Image Pro Plus version 3.0 software (Diagnostic Instruments Inc., Sterling Heights, Mich.). In order to visualize and compare the distribution and relative densities of ADH, pseudo-colored density maps were prepared for each micrograph immunostained for ADH. First, all colors except reds were removed from the digital images using Adobe Photoshop (selective color: reds) (San Jose, Calif.). The resulting images were saved and opened in ImageJ. Each image was then pseudo-colored using a calibrated equally-spaced 16-color look-up table (Rasband, 1997).

Distribution of ADH within Normal and Neoplastic Prostate Tissue

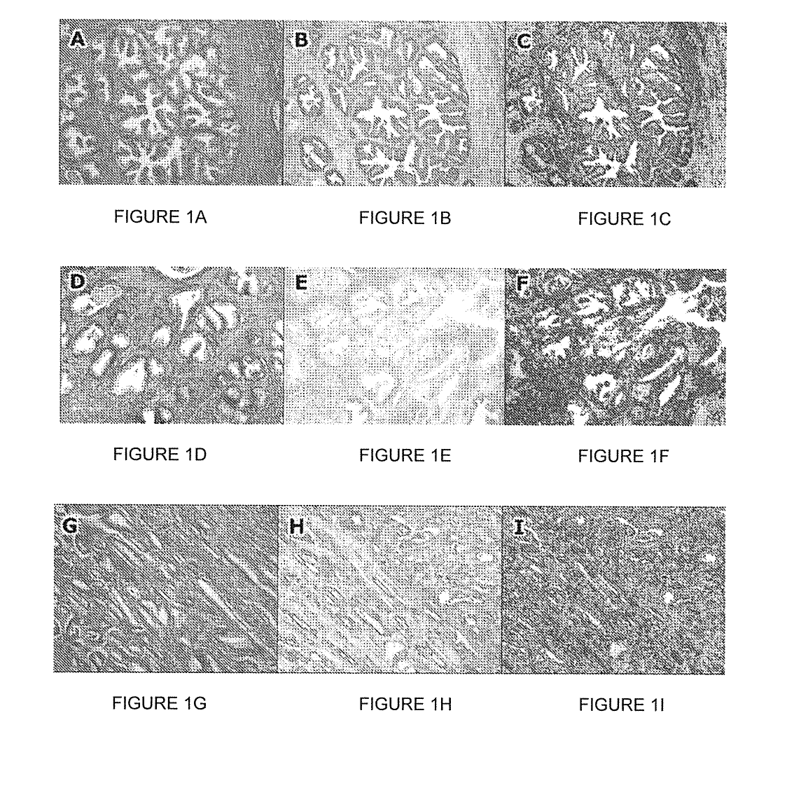

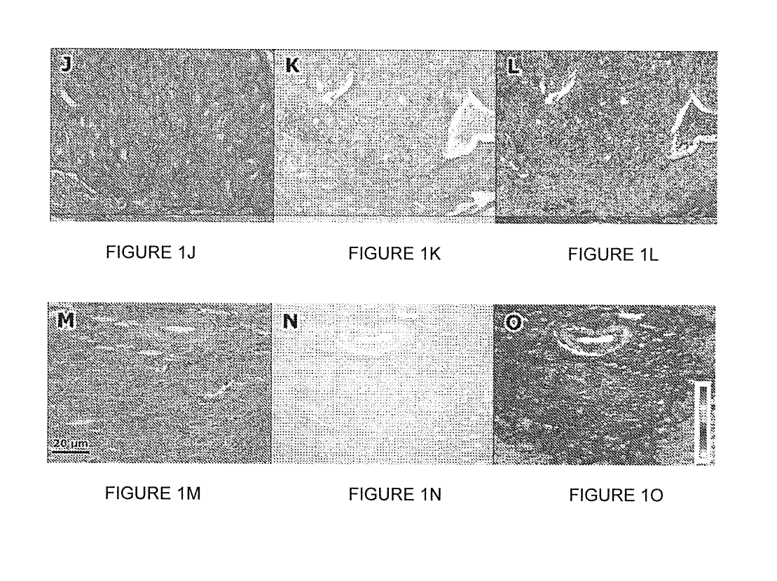

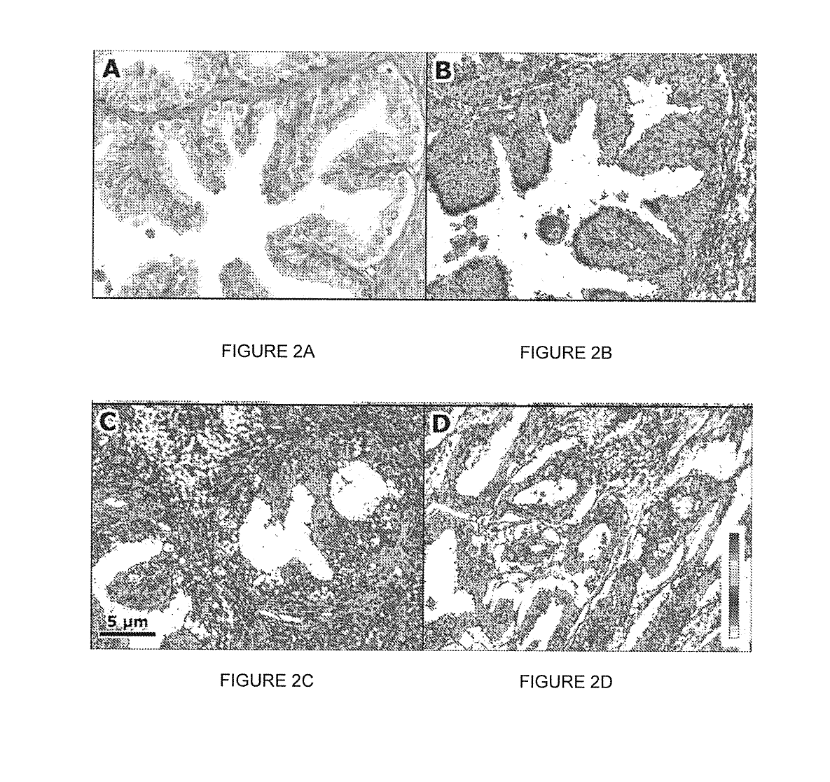

FIGS. 1A-10 show results of histology, immunocytochemistry and density analysis of normal and neoplastic human prostate tissue. FIGS. 1A, 1D, 1G, 1J and 1M show tissue samples stained with H&E; FIGS. 1B, 1E, 1H, 1K and 1N show results of immunocytochemistry using an antibody cross-reactive with class 1 and class 4 ADH; and FIGS. 1C, 1F, 1J, 1L and 1O show density analysis of ADH immunostaining. FIGS. 1A-1C show normal prostate tissue; FIGS. 1D-1F show BPH tissue; FIGS. 1G-1I show Gleason pattern 3 tissue; FIGS. 1J-1L show Gleason pattern 4 tissue; and FIGS. 1M-1O show Gleason pattern 5 tissue. All of the images in FIGS. 1A-1O were taken at a magnification of 100×. FIGS. 2A-2D show results of histology and density analysis of normal and neoplastic human prostate parenchyma. FIG. 2A shows a tissue sample stained with H&E; FIGS. 2B-2D show density analysis of ADH 1/4 immunostaining. Samples in FIGS. 2A and 2B are of normal prostate tissue. The sample in FIG. 2C is BPH tissue and the sample in FIG. 2D is Gleason pattern 3 tissue. All of the images in FIGS. 2A-2D were taken at a magnification of 400×.

The branching duct-acinar glandular system that is characteristic of prostate tissue was observed in normal samples as well as in cancer samples at locations distant from the cancer site, as shown in FIGS. 1A-1C. Glands were embedded in a fibromuscular stroma whose density varied with prostatic zone. Age-related, atrophic changes were observed throughout all glandular regions of normal tissue and were considered benign. Luminal and basal epithelial cells lining the acini and ducts were immunopositive for ADH. Density analysis associates the most intense ADH immunostaining with epithelium of acini and ducts. In many acini, the density of immunostaining appears as a gradient from the basement membrane through the basal cell layer to the apical membrane of the luminal epithelium, as shown in FIGS. 2A and 2B. Intense ADH immunostaining was also located in the endothelial lining of blood vessels embedded in the glands and stroma. A less intense immunostaining was observed in the smooth muscle of the stroma. The overall pattern of ADH immunostaining remained consistent throughout normal tissue regardless of gland morphology and stroma density in the three prostatic zones.

A variable amount of hyperplastic epithelium and stroma was observed in BPH samples as shown in FIGS. 1D-1F. The size and shape of the acini are irregular and lumina are variously enlarged with papillary projections. The epithelium has a columnar appearance with the nuclei crowded at the basal membrane. The density of ADH immunostaining in BPH samples is markedly reduced in the epithelium and absent in some acini as shown in FIG. 2C. Although immunostaining is evident in the stroma; density analysis indicates a reduction in overall intensity. Endothelial linings of blood vessels in BPH samples immunostained at densities similar to those in normal tissue.

Neoplastic samples encompassed Gleason patterns 3, 4, and 5 with Gleason scores ranging from 6 through 9. Samples with Gleason pattern 3 have medium sized glands of irregular shape and spacing with clear stromal separation, as shown in FIGS. 1G-1I. ADH immunostaining in these samples is associated with acini epithelium although density analysis indicates the intensity of staining is reduced compared to normal epithelium. The gradient of immunostaining observed in normal tissue is also absent; immunostaining in luminal cells does not appear to be denser at the apical membrane, as shown in FIG. 2D. However, the normal glands dispersed among neoplasms maintain strong ADH expression. ADH immunostaining in stromal tissue surrounding neoplastic glands does not appear to differ from normal stroma.

Poorly formed glands with undefined lumina are observed in samples with Gleason pattern 4, as shown in FIGS. 1J-1L. The loss of glandular architecture and organization is associated with an irregular distribution of ADH-expressing epithelium. ADH immunostaining appears punctuate in that some epithelial cells are immunoreactive, but neighboring cells within a given acinar gland are not. Little or no glandular differentiation is observed in samples with Gleason pattern 5, as shown in FIGS. 1M-1O. ADH immunostaining is absent except in scattered stromal cells. Immunostaining associated with blood vessels is also absent.

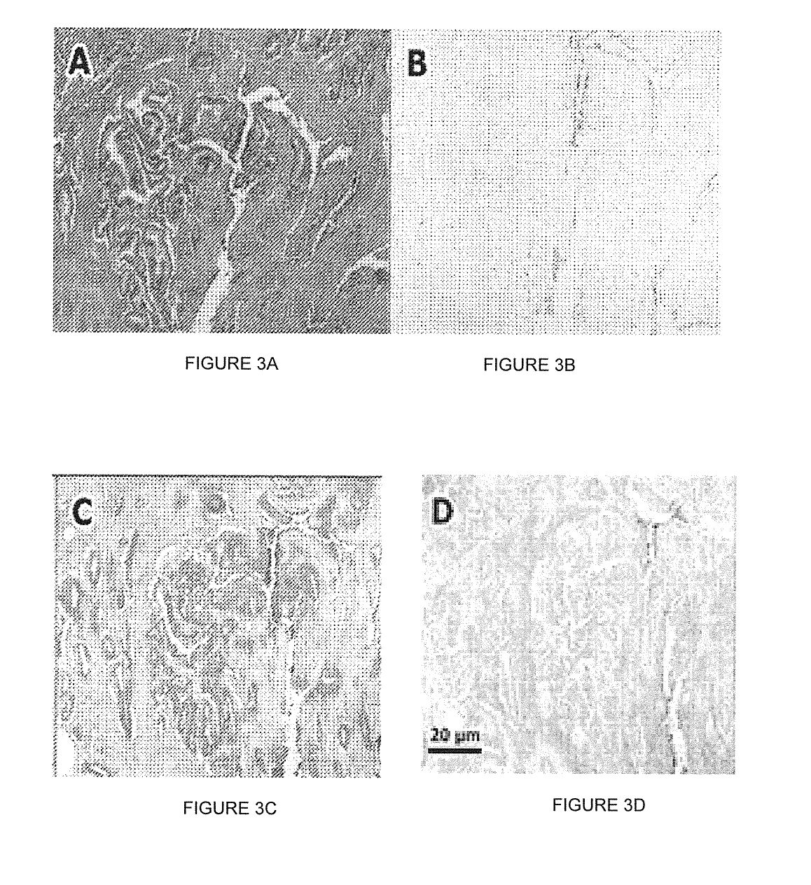

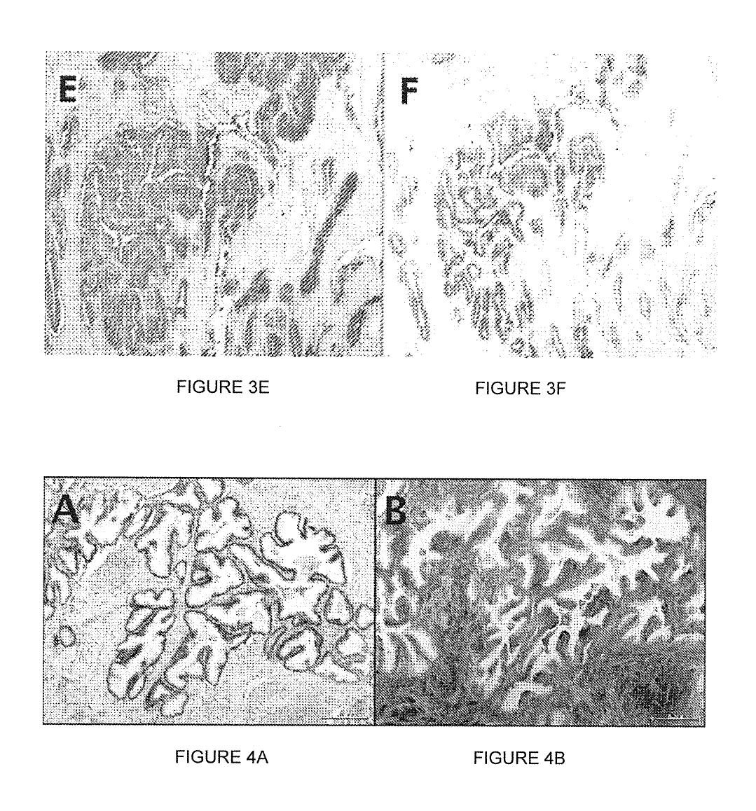

FIGS. 3A-3F show serial sectioned controls for immunocytochemistry of ADH. FIG. 3A shows Gleason pattern 3 tissue stained with H&E; FIG. 3B shows a no-antibody control incubated with PBS instead of ADH antibody; and FIG. 3C shows tissue immunostained with an antibody which recognizes ADH 1/4. Epithelial differentiation markers immunostain these serial sections as expected: FIG. 3D shows AR immunostains both acini epithelium and stroma, FIG. 3E shows PSA immunostaining and FIG. 3F shows CK18 immunostaining of the acini and ductal epithelium. All of the images in FIGS. 3A-3F were taken at a magnification of 100×.

Negative controls run on all samples demonstrate the specificity of the ADH antibody, as shown in FIGS. 3B and 3C. Antibodies to known epithelial differentiation markers (AR, PSA, and CK18) confirmed the integrity of the ADH immunostaining, as shown in FIGS. 3D-3F. Immunostaining for AR is associated with both epithelium and stroma whereas immunostaining for PSA and CK18 is associated with acini epithelium.

Significant down-regulation of ADH is observed in BPH samples from both races as indicated by density analysis of immunoreactive protein and quantitation of ADH mRNAs.

Distribution of ADH Relative to 34 Beta E12 in Normal and Neoplastic Prostate Tissue

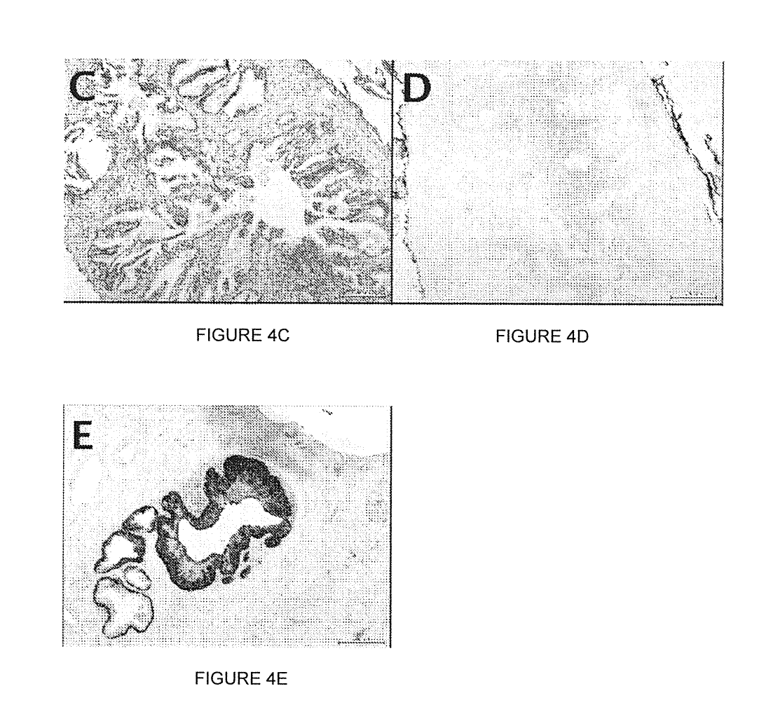

FIGS. 4A-4E show results of immunocytochemistry using antibodies specific for ADH1B or 34 Beta E12 in BPH tissue. FIGS. 4A and 4C show images of BPH tissue immunostained with antibody specific to 34 Beta E12 and counterstained with hematoxylin QS. FIGS. 4B and 4D show images of BPH tissue immunostained with antibody specific to ADH1B. FIG. 4E is an image of BPH tissue double immunostained with ADH1B and 34 Beta E12 antibodies. FIGS. 4A and 4B are serial sections and exhibit glandular hyperplasia with coincidence of high ADH1B reactivity and 34 Beta E12 reactivity. FIGS. 4C and 4D are serial sections that exhibit coincidence of low ADH1B and low 34 Beta E12 reactivity. Tissues shown in FIGS. 4A and 4B and FIGS. 4C and 4D come from different sources but have identical clinical diagnoses of BPH with inflammation. Immunostaining with ADH1B indicates that these two BPH samples belong to different subpopulations of BPH. FIG. 4E shows results of immunostaining with ADH1B and 34 Beta E12 shows basal cell hyperplasia distant from PCa. Cell proliferation alone is not sufficient to reduce ADH1B levels.

Variation in the density of ADH immunostaining was observed in samples with the clinical diagnosis of BPH, BPH with chronic inflammation, or BPH with metaplasia and prostatitis. The intense ADH1B immunostaining associated with the epithelium of acini and ducts observed in normal tissue was also observed in some of the BPH samples, as shown in FIG. 4A. The ADH1B immunostaining pattern in other BPH samples, however, was similar to that observed in PCa samples with little or no ADH1B expression, as shown in FIG. 4C. The common clinical diagnosis of these BPH samples was not predictive of the intensity of the ADH1B immunostaining.

An antibody, 34 beta E12, was used to immunostain both BPH and PCa samples. Specific for the high molecular weight cytokeratins 1, 5, 10, and 14, the 34 beta E12 antibody identifies basal cells within the prostate gland. Prostatic basal cells reside on the basement membrane and are essential in maintaining normal differentiation of luminal cells and integrity of prostatic ducts. The loss of basal cells is characteristic of PCa (Goldstein et al., Science. 2010; 329(5991):568-571). Using this antibody it was found that ADH1B immunostaining was stratifying the BPH population into two distinct groups: one group with high ADH1B expression and abundant 34 beta E12 immunostaining indicating numerous basal cells, as shown in FIGS. 4A and 4B, and a second group with no ADH1B expression and little 34 beta E12 immunostaining indicating few, if any, basal cells, as shown in FIGS. 4C and 4D. An additional sample of basal cell hyperplasia was double stained with both ADH1B and 34 beta 12 antibodies, as shown in FIG. 4E. The strong expression of both ADH1B and 34 beta 12 in this sample indicates that cell proliferation alone is not associated with loss of ADH1B expression.

Nucleic Acid Extraction from FFPE Samples

Total RNA was extracted from all FFPE samples (ten 8 μm sections) using Recover All™ Total Nucleic Acid Isolation kit (Ambion, Inc.). Samples were treated with 1 ml of xylene, mixed and incubated for 3 minutes at 50° C. to remove the paraffin. Following centrifugation at 14,000×g for 2 minutes, samples were washed twice with 1 ml of 100% ethanol and allowed to air dry. For RNA isolation, 400 μl of Digestion Buffer and 4 microliters (μl) of protease were added to each sample and incubated for 3 hours at 50° C. For DNA isolation, 400 μl of Digestion Buffer and 4 μl of protease were added to each sample and incubated at 50° C. for 48 hours. For both DNA and RNA isolation, 480 μl of Isolation Additive and 1.1 ml of ethanol were added to each sample. The samples were then passed through a Filter Cartridge. Following filtration, the flow through was discarded and 700 μl of Wash Buffer were passed through the cartridge. Each filter containing a RNA sample was treated with DNase for 30 minutes at 37° C. whereas each filter containing a DNA sample was treated with RNase for 30 minutes at 37° C. All filters were then washed with 700 μl of Wash 1, centrifuged, and the flow-through discarded. Filters were then washed a second time with 500 μl of Wash Buffer 2. The nucleic acid samples were then eluted from the filters using 30 μl of Elution Buffer. The resulting RNA was stored at −70° C. prior to analysis.

Isolation of RNA from Urine Samples

Thirty to ninety milliliters (ml) of urine were collected from each participant in an AssayAssure 90 ml urine specimen cup (ThermoScientific). Urine was processed within 18 hours of collection for RNA extraction using ZR Urine RNA isolation kit (Zymo Research Laboratories). Each sample was mixed to suspend any cells that may have settled to the bottom of the specimen cup. Cells were isolated from the urine sample by drawing 60 ml of urine into a syringe fitted with a ZRC GF filter. The urine was then passed through the filter using slow, steady pressure. The filtrate was discarded. The sample urine was removed completely from the filter by pushing through several volumes of air. Using a 1 ml syringe, 700 μl of Urine RNA Buffer was pushed through the filter and the filtrate collected in an RNase-free 1.5 ml microcentrifuge tube. 700 μl of 100% ethanol was added to the filtrate tube and mixed. The sample was then transferred to a Zymo-Spin IC column and centrifuged at 12,000×g for 1 minute. The column filtrate was discarded. 400 μl of RNA Prep Buffer was added to the column and centrifuged at 12,000×g for 1 minute. The column filtrate was discarded. 700 μl of RNA Wash. Buffer was added to the column and centrifuged at 12,000×g for 1 minute. The wash was then repeated using 400 μl of RNA Wash Buffer. The column was placed in a RNase-free microcentrifuge tube and 10 μl nuclease free water was added to the column. Following 1 minute of incubation, the column was then centrifuged at 10,000×g for 1 minute to collect the RNA. The resulting RNA was stored at −70° C. prior to analysis.

Reverse Transcription and Gene Amplification of FFPE and Urine Samples

2 micrograms (μg) of total RNA from each RNA sample was reverse transcribed using TaqMan High Capacity RNA to cDNA Reverse Transcription Reagents (Applied BioSystems, Foster City, Calif.) to obtain a 0.05 μg/μl stock of cDNA. Target sequences were then selectively amplified in each sample using TaqMan Gene Expression PreAmplification Master mix (Applied BioSystems).

Real time quantitative PCR (qRT-PCR) reactions were run using Gene Expression Master Mix (Applied Biosystems) and 4 ng of preamplified sample cDNA in 20 μl reactions. Main Line Health FFPE sample reactions were incubated in a Stratagene MX3005P (La Jolla, Calif.) and FFPE samples obtained from Karmanos Cancer Center and urine samples were incubated in an Applied Biosystems StepOnePlus (Foster City, Calif.). All samples were run with 1 cycle at 50° C. for 1 minute, 1 cycle 95° C. for 10 minutes, followed by 40 cycles of alternating 95° C. for 15 seconds and 60° C. for 60 seconds. Analysis of expression for Main Line Health FFPE samples was done using MxPro software version 4.01 (Stratagene). All other samples were analyzed using StepOnePlus software version 2.1 (Applied Biosystems). Expression of each gene was quantified relative to expression of the normal calibrator. RNA obtained from the normal Caucasian male was used as the calibrator sample for all assays (PN_{CAU}).

For all FFPE samples, the mean relative expression of each gene was determined from the qRT-PCR results by measuring expression in two duplicate assays, each of which was measured in triplicate. To determine the inter-assay variation, a two-tailed t-test for independent samples was used to compare means of the triplicate measurements between the duplicate assays. Duplicate assays showing no significant difference between means (p>0.05) were pooled to yield the mean relative gene expression (M_{pooled}). All qRT-PCR data are expressed as the mean (+/− the standard error of the mean).

The M_{pooled} for all genes were arcsin square-root transformed prior to statistical analysis. A one-way analysis of variance (ANOVA; n=23) was used to test for effects of disease state on gene expression without regard to race. Comparisons were made between all normal samples and all BPH samples, all normal samples and all cancer samples (regardless of Gleason score), as well as all BPH samples and all cancer samples (regardless of Gleason score).

A two-way ANOVA was run to test for effects of both race and disease state. PN_{CAU} was compared to BPH, Gleason scores 6/7 and Gleason scores 8/9. Post hoc comparisons of BPH samples to cancer samples were done using a Tukey test. Contingency tables were used to analyze differences in ADH4 expression between the races.

Laser Dissection of FFPE Samples

Samples illustrative of BPH and Gleason scores 6 through 9 were selected for their visual presentation pattern and their suitability for dissecting out individual components. Four patient samples were dissected into normal epithelium, cancerous epithelium, and stroma to confirm the distribution of ADH expression within each sample. An additional three patient samples with extensive disease were dissected into tumor and stroma. For these samples, no normal epithelium could be located. Six 8 μm sections of each sample were mounted on Laser Dissection Membrane slides (Molecular Machines & Industries, Zurich, Switzerland) under RNAse-free conditions. Mounted samples were deparaffinated and stained lightly with Harris Hematoxylin (Electron Microscopy Sciences, Fort Washington, Pa. Laser dissection of each sample was carried out using CellTools V2.3 on a CellCut UV Laser system (Molecular Machines & Industries) with Sony Exwave HAD camera (San Diego, Calif.) on an Olympus IX71 microscope. Dissected areas of interest were transferred to Adhesive Lid with Diffuser microcentrifuge tubes (MMI) and processed for expression of ADH1B. ADH1C, ADH4, and RPLPO using qRT-PCR as described for undissected samples. PN_{CAU} was used as the calibrator sample for all dissected assays. Expression of the epithelial marker AR was used as a positive control and expression of RPLPO was used as a normalizing endogenous control.

A two-tailed t-test was used to compare the expression of ADH1B in hyperplastic epithelium to expression of ADH1B in morphologically normal epithelium dissected from the same sample. ADH1B expression in dissected tumor was also compared to ADH1B expression in morphologically normal epithelium dissected from the same sample. A two-tailed t-test was used to compare expression of ADH1B in morphologically normal epithelium of dissected cancer samples to morphologically normal epithelium dissected from PN_{CAU}. In addition; ADH1B expression in tumor was compared to ADH1B expression in morphologically normal epithelium dissected from PN_{CAU}.

Quantitative Real Time RT-PCR

Taqman Gene Expression Assays (Applied Biosystems, Foster City, Calif.) were used to determine expression of ADH1A, ADH1B, ADH1C, ADH4, HNF1B, AR, PSA, and CK18. Target regions of each nucleic acid specific for the indicated isoform are shown in Table IIA. Exemplary probes and primers for assay of the indicated biomarker are shown in Table IIB.

Table IIA
Identification of amplicon sequence for primers & probes used for qRT-PCR

| Gene | NCBI ref. | Assay | Amplicon | |
|---|---|---|---|---|
| Symbol | Sequence | Location | Length | Amplicon Region |
| ADH1A | NM_000667.3 | 1081 | 75 | tgctactgactggacgtacctggaagggagctattcttggtggc |
| | | | | tttaaaagtaaagaatgtgtcccaaaacttgtggctgattttatg |
| | | | | SEQ ID NO: 2 |
| ADH1B | NM_000668.4 | 1043 | 115 | tcgtaggggtacctcctgcttcccagaacctctcaataaacccta |
| | | | | tgctgctactgactggacgcacctggaaggggctgtttatggt |
| | | | | ggctttaagagtaaagaaggtatcccaaaacttgtggctgatttt |
| | | | | atggctaagaagttttcactggatgcgttaataac |
| | | | | SEQ ID NO: 3 |
| ADH1C | NM_000669.3 | 1192 | 225 | aaccctatgctgctactgactggacgcacgtggaaaggagcta |
| | | | | tttttggaggctttaagagtaaagaatctgtccccaaacttgtggc |
| | | | | tgactttatggctaagaagttttcactggatgcattaataacaaat |
| | | | | attttaccttttgaaaaaataaatgaaggatttgacctgcttcgctc |
| | | | | tggaaagagtatccgtaccgtcctgacgttttgaaacaatacag |
| | | | | atgccttcccttgtagcagttttcagcctcctctaccctacatgatc |
| | | | | tggagcaacagctaggaaatatcattaattctgctcttcagagat |
| | | | | gttaaaaataaattacacgtgggagctttccaaagaaatggaaa |
| | | | | ttgatgggaaattatttgtcaagcaaa |
| | | | | SEQ ID NO: 4 |
| ADH4 | NM_000673.4 | 844 | 57 | ctggatatggcgctgctgttaaaactggcaaggtcaaacctggt |
| (aka | | | | tccacttgcgtcgtctttggcctg |
| ADH7) | | | | SEQ ID NO: 5 |
| HNF1B | NM_000458.2 | 562 | 95 | aaggagctgcaggcgctcaacaccgaggaggeggcggagc |
| | | | | agcgggcggaggtggaccggatgctcagtgaggacccttgg |
| | | | | agggctgctaaaatgatcaagggttacatgcagcaacacaaca |
| | | | | tcccc |
| | | | | SEQ ID NO: 6 |
| AR | NM_000044.3 | 3001 | 99 | attccgaaggaaaaattgtccatcttgtcgtcttcggaaatgttat |
| | | | | gaagcagggatgactctgggagcccggaagctgaagaaactt |
| | | | | ggtaatctgaaactacaggaggaaggagaggcttccagcacc |
| | | | | accagcc |
| | | | | SEQ ID NO: 7 |
| PSA | NM_ | 253 | 65 | gtcctcacagctgcccactgcatcaggaagccaggtgatgact |
| (KLK3) | 001030048.1 | | | ccagccacgacctcatgagctccgc |
| | | | | SEQ ID NO: 8 |
| CK18 | NM_000224.2 | 531 | 64 | attacttcaagatcatcgaggacctgagggctcagatcttcgca |
| (K18) | | | | aatactgtggacaatgcccgcat |
| | | | | SEQ ID NO: 9 |

TABLE IIB
Example of Primers and probes for: ADH1A,
ADH1B, ADH1C, ADH4, HNF1B, AR, PSA, and CK18.

| Gene | Examples | | |
|---|---|---|---|
| Symbol | of Primer Set and probe | | |
| ADH1A | Forward: | cgtacctggaagggagctattc | SEQ ID |
| | | | NO: 10 |
| | Reverse | gccacaagttttgggacaca | SEQ ID |
| | | | NO: 11 |
| | Probe: | tggtggctttaaaagt | SEQ ID |
| | | | NO: 12 |
| ADH1B | Forward: | tgactggacgcacctggaa | SEQ ID |
| | | | NO: 13 |
| | Reverse | gttattaacgcatccagtgaaa | SEQ ID |
| | | ac | NO: 14 |
| | Probe: | ttatggtggctttaagagtaa | SEQ ID |
| | | | NO: 15 |
| ADH1C | Forward: | aaggagctatttttggaggctt | SEQ ID |
| | | taa | NO: 16 |
| | Reverse | agaggaggctgaaaactgctac | SEQ ID |
| | | a | NO: 17 |
| | Probe: | ttatggtggattaagagtaa | SEQ ID |
| | | | NO: 18 |
| ADH4 | Forward: | ggatatggcgctgctgttaaa | SEQ ID |
| (aka | | | NO: 19 |
| ADH7) | Reverse | gacgacgcaagtggaacca | SEQ ID |
| | | | NO: 20 |
| | Probe: | ctggcaaggtcaaac | SEQ ID |
| | | | NO: 21 |
| HNF1B | Forward: | aacaccgaggaggcggc | SEQ ID |
| | | | NO: 22 |
| | Reverse | ctgcatgtaacccttgatcat | SEQ ID |
| | | ttta | NO: 23 |
| | Probe: | gaccggatgctcagtga | SEQ ID |
| | | | NO: 24 |
| AR | Forward: | tgtcgtcttcggaaatgttat | SEQ ID |
| | | ga | NO: 25 |
| | Reverse | ggaagcctaccttcctcct | SEQ ID |
| | | | NO: 26 |
| | Probe: | actctgggagcccgg | SEQ ID |
| | | | NO: 27 |
| PSA | Forward: | acagctgcccactgcatc | SEQ ID |
| | | | NO: 28 |
| | Reverse | aggcggagcagcatgag | SEQ ID |
| | | | NO: 29 |
| | Probe: | ccaggtgatgactcc | SEQ ID |
| | | | NO: 30 |
| CK18 | Forward: | ccattacttcaagatcatcgag | SEQ ID |
| | | gac | NO: 31 |
| | Reverse | gggcattgtccacagtatttgc | SEQ ID |
| | | | NO: 32 |
| | Probe: | tgagggctcagatct | SEQ ID |
| | | | NO: 33 |

Androgen receptor (AR), prostate specific antigen (PSA), and cytokeratin (CK18) expression were measured as markers for epithelial differentiation in PCa and served as positive assay controls. Ribosomal protein large (RPLPO) expression was used as a normalizing endogenous control. Standard curves were run to validate RPLPO expression as an endogenous control using First Choice Normal Prostate RNA (C_{Norm}) and First Choice Tumor Prostate RNA (C_{Tumor}: Ambion, Inc. Austin, Tex.). The C_{T} values generated from equivalent standard curve mass points were used to calculate the difference between the C_{T} RPLPO normal and C_{T} RPLPO tumor (ΔC_{T}). The resulting ΔC_{T} values were plotted against the log of the input RNA.

Probes

MGB (minor groove binding) probes from the collection of Applied Biosystem Assays on Demand Gene Expression Assays can be used:

SEQ ID NO: 34
ADH4: AACTGGCAAGGTCAAACCTGGTTCC;
SEQ ID NO: 35
ADH1B: GGGGGCTGTTTATGGTGGCTTTAAG;
SEQ ID NO: 36
ADH 1C: TGGAAAGAGTATCCGTACCGTCCTG;

Analysis of ADH Isozyme Expression in Normal and Neoplastic Prostate Tissue

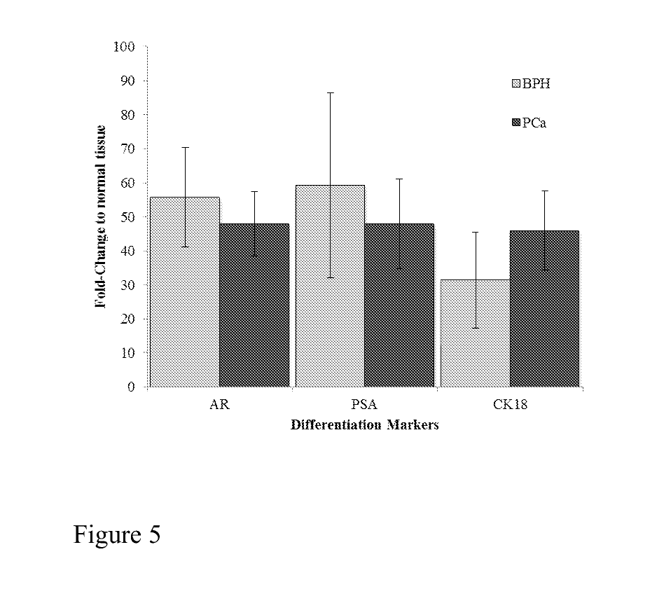

FIG. 5 shows expression of epithelial differentiation markers AR, PSA and CK18 in BPH and PCa samples (±SEM). RPLPO was the endogenous control and PN_{CAU} was the normal calibrator. Expression of all markers is significantly elevated relative to expression in normal prostate tissue.

Expression of all epithelial differentiation markers, AR, PSA, and CK18, indicated that the profile of the patient samples was consistent with a clinical diagnosis of BPH or cancer. The expression of AR, PSA, and CK18 was elevated in both BPH and PCa samples, as shown in FIG. 5.

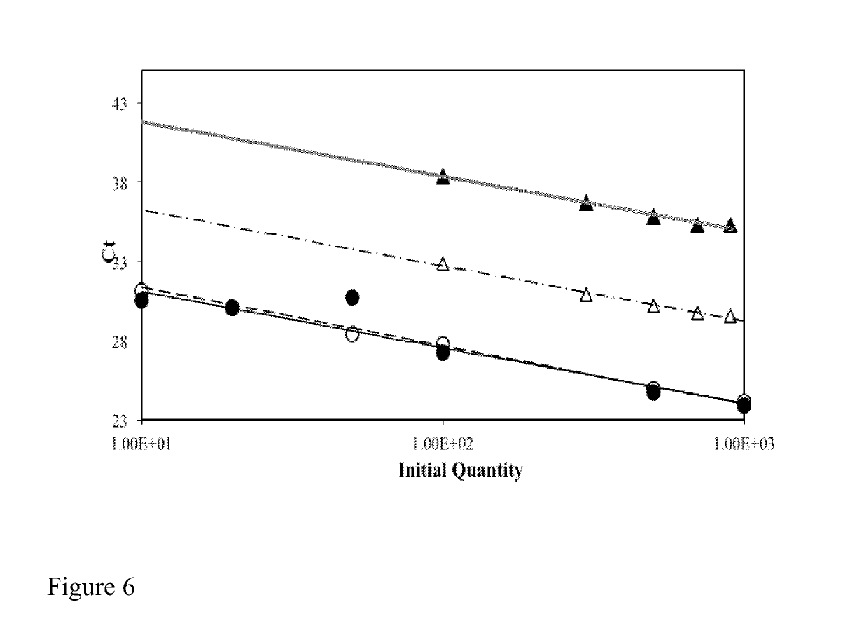

RPLPO expression was validated for use as a qRT-PCR endogenous control using ΔC_{T} values derived from the standard curves of normal and tumor RNA. FIG. 6 shows validation of RPLPO endogenous control used for qRT-PCR analysis of FFPE samples. cDNA reverse transcribed from RNA extracted from normal and PCa tissue was used to generate standard curves with ADH1B and RPLPO probes. Expression of ADH1B differs between normal (open triangle) and PCa (closed triangle) samples whereas expression of RPLPO is consistent between normal (open circle) and PCa (closed circle) samples. To account for experimental variation between samples, ADH expression in all samples was normalized to RPLPO expression. The slope of ΔC_{T} versus log of input RNA was <0.1, indicating RPLPO expression does not differ between normal and tumor prostate RNA, as shown in FIG. 6.

The profile of ADH gene expression was determined using probes that distinguish between the different ADH gene products in a qRT-PCR assay. In C_{Norm}, expression of ADH1B accounted for 97.8% (+/−0.5) and ADH1C accounted for 2.2% (+/−0.5) of the total ADH expression measured. ADH1A and ADH4 expression were not detected. This expression pattern was also observed in RNA extracted from the FFPE normal samples PN_{AA} (ADH1B=98.2%+/−0.06) and PN_{CAU} (ADH1B=98.3%+/−0.04).

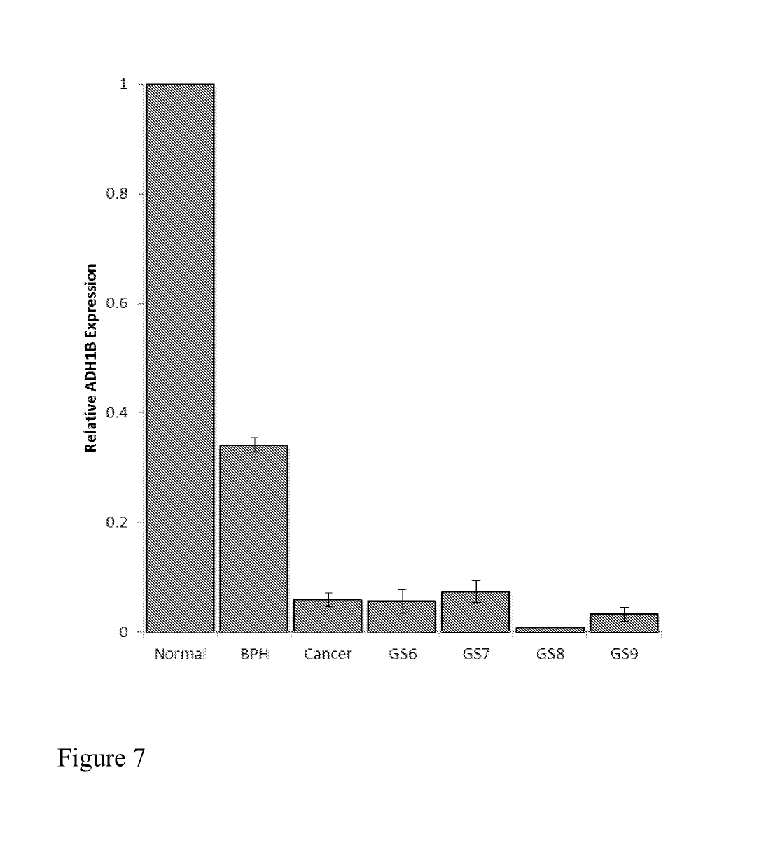

FIG. 7 shows expression of ADH1B in normal, BPH, and PCa FFPE (±SEM). RPLPO was the endogenous control and PN_{CAU} was the normal calibrator. ADH1B expression is significantly down-regulated from normal in BPH samples. Expression in all Gleason scores (GS6-GS9) is significantly lower than both normal and BPH and there is no significant difference in ADH1B expression between the Gleason scores.

ADH1B is significantly down-regulated (p<0.05) in BPH samples to 0.34 fold (+/−0.013) of normal expression, as shown in FIG. 7. In all neoplastic samples ADH1B is significantly down-regulated (p<0.05) from expression in both BPH and normal samples. The down-regulation of ADH1B in cancer samples is independent of the specific Gleason score (p>0.259; FIG. 7).

ADH1B expression in hyperplastic epithelium dissected from BPH is significantly down-regulated (p<0.0001) to 0.028 fold (+/−0.0059) of ADH1B expression in morphologically normal epithelium dissected from a normal sample (PN_{CAU}). Morphologically normal epithelium dissected from cancer samples (N=3) is significantly down-regulated (p<0.0001) to 0.009 fold (+/−0.007) of ADH1B expression in dissected PN_{CAU} epithelium. ADH1B expression in neoplastic epithelium dissected from tumor (N=3) is significantly down-regulated (p<0.0001) to 0.0007 fold (+/−0.007) of epithelium dissected from PN_{CAU}. Expression of ADH1B in stroma dissected from BPH samples (N=2) does not differ from ADH1B expression in stroma of PN_{CAU} (p>0.75).

Analysis of HNF1B Expression in Normal and Neoplastic Prostate Tissue

HNF1B expression was measured in 21 FFPE samples; 10 samples tested were samples of BPH and 8 samples were BPH with concurrent benign diseases such as inflammation and metaplasia, the remaining 3 samples were PCa. The mean relative expression for HNF1B in samples with BPH is 3.66 fold (+/−1.13) of normal expression and did not differ from expression of HNF1B in BPH samples with concurrent benign disease the mean expression of which is 2.59 fold (+/−0.70) of normal expression. Expression of HNF1B in samples with PCa is 8.52 fold (+1-1.80) of normal expression, which is significantly higher than expression in samples with BPH alone and BPH with concurrent disease (p<0.02).

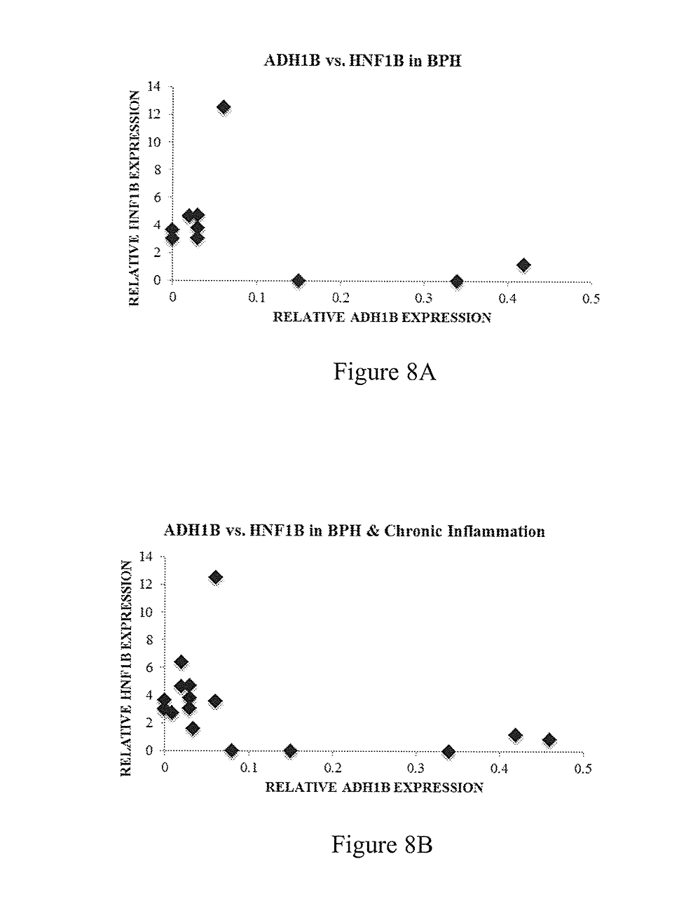

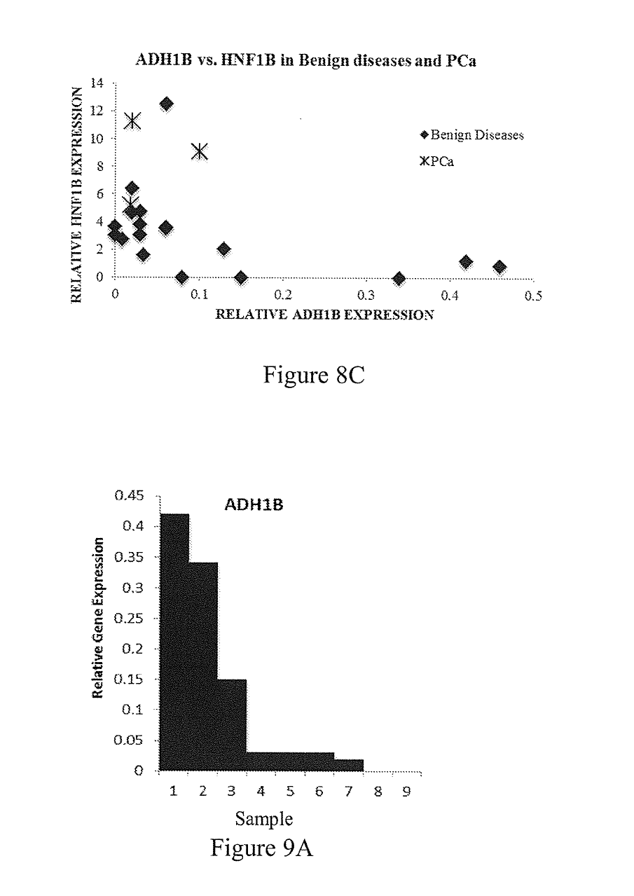

FIGS. 8A-8C show graphs of expression of ADH1B and HNF1B in FFPE samples with BPH, BPH with Chronic Inflammation, and PCa. Ribosomal protein large (RPLPO) expression was used as a normalizing endogenous control. All qRT-PCR data are expressed as the mean (+/− the standard error of the mean). FIG. 8A is a graph showing expression of ADH1B and HNF1B in FFPE tissue samples with BPH alone. FIG. 8B is a graph showing expression of ADH1B and HNF1B in FFPE tissue samples with BPH, BPH with chronic inflammation and/or prostatitis. FIG. 8C is a graph showing expression ADH1B and HNF1B expression in FFPE samples with BPH, BPH with chronic inflammation, prostatitis or metaplasia, or malignant PCa. Expression of ADH1B and HNF1B stratify the BPH samples into two distinct groups: samples expressing high levels of ADH1B and low levels of HNF1B, or samples expressing high levels of HNF1B and low levels of ADH1B. Samples with PCa have low levels of ADH1B and high levels of HNF1B.

The BPH samples stratify into two distinct expression patterns: high ADH1B expression paired with low HNF1B expression, and low ADH1B expression paired with high HNF1B expression, as shown in FIGS. 8A-8C. All PCa samples cluster with the low ADH1B/high HNF1B expression BPH group.

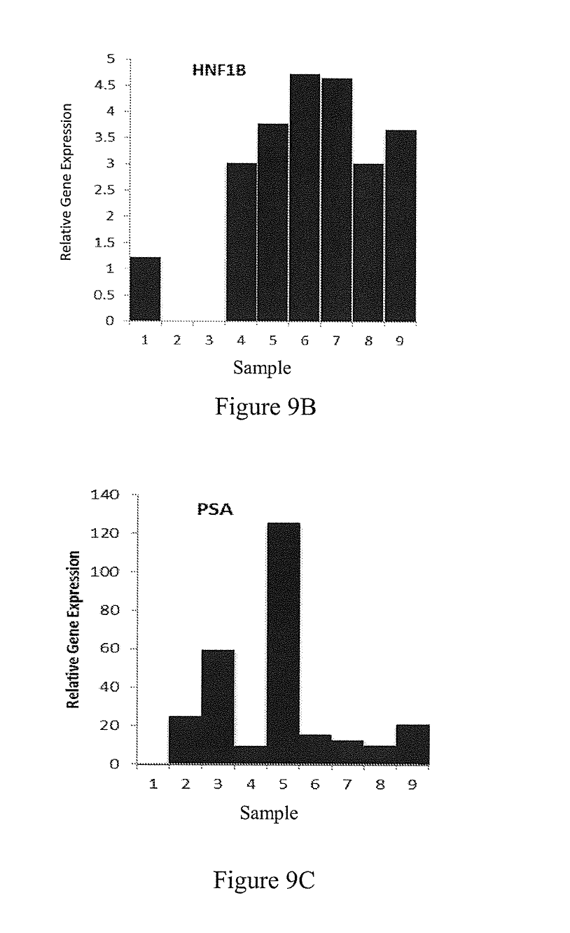

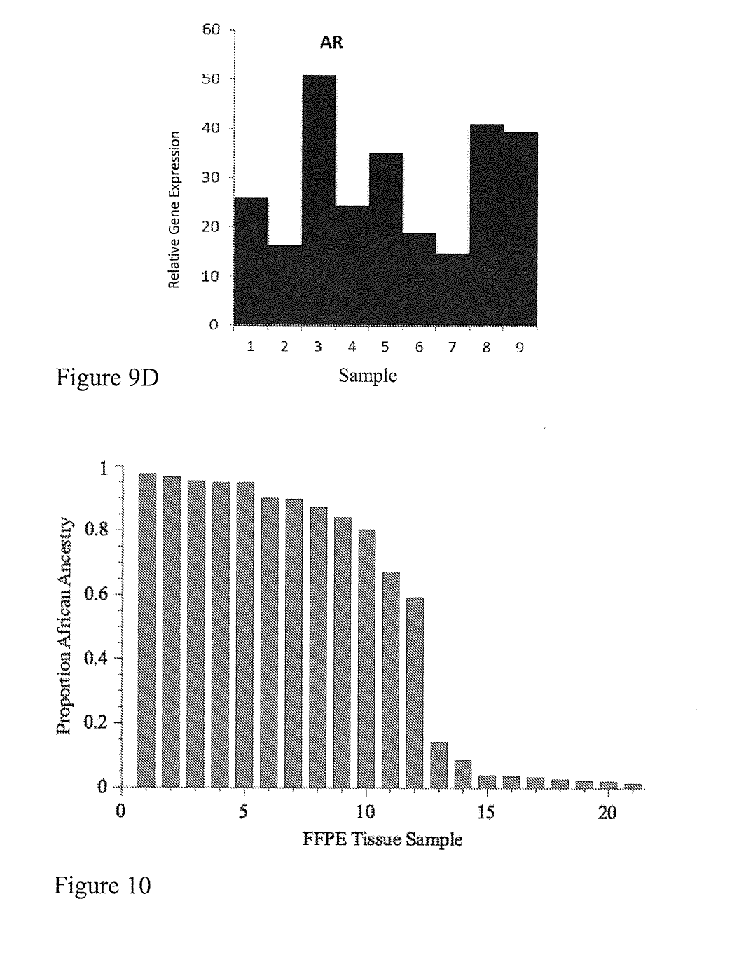

FIGS. 9A-9D show graphs of relative expression of ADH1B, HNF1B, AR and PSA in 9 BPH samples as measured by qRT-PCR. FIG. 9A is a graph showing relative ADH1B expression in 9 BPH samples as measured by qRT-PCR; FIG. 9B is a graph showing relative HNF1B expression in corresponding 9 BPH samples as measured by qRT-PCR; FIG. 9C is a graph showing relative AR expression in corresponding 9 BPH samples as measured by qRT-PCR; FIG. 9D is a graph showing relative PSA expression in corresponding 9 BPH samples as measured by qRT-PCR. Ribosomal protein large (RPLPO) expression was used as a normalizing endogenous control. All qRT-PCR data in FIGS. 9A-9D are expressed as the mean (+/− the standard error of the mean). Expression of ADH1B and HNF1B exhibit an inverse relationship; as ADH1B decreases in the sample population, HNF1B increases. Expression of AR and PSA has no discernible pattern when correlated to ADH1B expression.

Expression of AR and PSA do not correlate with expression of HNF1B or ADH1B. No subpopulations in either the BPH samples or PCa samples become apparent with either of these epithelial differentiation markers, as shown in FIG. 9.

The relative quantity of ADH1C expression in BPH was highly variable between samples and did not significantly differ from expression in normal tissue. In neoplastic samples, expression of ADH1C was significantly downregulated from normal tissue (p<0.01). Expression of ADH1C was 0.39 fold (+/−0.2) of normal in BPH samples and 0.12 fold (+/−0.05) of normal in neoplastic samples. ADH1C expression was not correlated with Gleason grade. Racial differences in ADH1C expression were not observed. ADH1A was not detected in either BPH or neoplastic samples.

ADH4 Expression in FFPE Samples

Analysis of ADH4 expression in 32 BPH samples and 22 PCa samples revealed a highly significant racial difference (p<0.0002) in the pattern of ADH isozyme expression. ADH4 was expressed in 58.8% (20 of 34 tested) of the samples from African-American men and 13% (4 of 30 tested) of samples from Caucasian men. For those samples in which ADH4 expression was detected, there was no significant racial difference in the level of expression (p>0.49). Assuming non-equal sample variance, the expression of ADH4 in BPH samples was significantly higher than ADH4 expression in PCa (p<0.028).

No racial difference in expression of ADH1B (p>0.15) was detected. The down-regulation of ADH1B did not differ between African-American patient samples that expressed ADH4 and those that did not. Additionally, down-regulation of ADH1B in these samples was not different from the down-regulation of ADH1B observed in Caucasian patient samples. The mechanisms regulating expression of ADH1B in these samples do not appear altered by the expression of ADH4. The presence of ADH4 in these samples may, however, influence and alter the metabolism of ADH substrates within prostate epithelium.

Expression of ADH4 in Urine

To determine the frequency of ADH4 expression in normal prostate tissue, 170 urine samples were collected from men under the age of 40 with no known history of prostatic disease. Total RNA was isolated from an aliquot of the urine sample and analyzed using qRT-PCR. ADH4 expression was detectable in 45 of 170 samples (26.5%). When analyzed by race, the number of African-American samples expressing ADH4 (17 of 54 or 31.5%) did not differ from the number of Caucasian samples expressing ADH4 (26 of 104 or 25.0%) (p>0.121). The remaining 12 samples were from men who identified themselves with ethnic groups other than African-American or Caucasian. Expression of ADH4 was detected in 2 of the 12 samples (16.7%).

The number of African-American BPH and PCa FFPE samples expressing ADH4 is significantly higher than the number of normal African-American urine samples expressing ADH4 (p<0.015). This over-representation of African-American samples in the BPH and PCa population indicates that expression of ADH4 is a risk factor for prostate cancer in African-Americans. The number of Caucasian BPH and PCa FFPE samples expressing ADH4 does not differ from the number of normal Caucasian urine samples expressing ADH4 (p>0.219).

The mean ADH4 expression observed in urine samples was 19.23 fold (+/−5.54) of the expression found in normal prostate tissue. Three samples had levels of ADH4 expression that were more than 2 standard deviations above the mean; one African-American sample, one Asian sample and one Caucasian sample. The mean ADH4 urine expression for these outlying samples was 143.258 fold (+/−37.12) of normal ADH4 expression in the prostate. Analysis of these 3 samples for ADH1B and HNF1B, revealed that these samples have the low ADH1B, high HNF1B expression profile that is consistent with that observed in samples with prostate cancer despite the fact that these men have no known history of prostatic disease. The mean ADH1B expression for these samples was 0.0012 (+/−0.0009) fold of normal prostate expression and the mean HNF1B expression was 38.79 (+7-9.22) fold of normal prostate expression.

Expression of ADH1B and HNF1B was also measured in 3 samples that did not express ADH4. For these samples, ADH1B was 2.48 (+7-1.43) fold of normal prostate expression and HNF1B was not detectable. The high ADH1B and low HNF1B profile of expression is consistent with the expression profile observed in the BPH cluster that is unlikely to progress to prostate cancer.

Thus, high expression of ADH4 in the urine identifies African-American men at a higher risk for developing prostatic disease while the prostate is still healthy. As shown herein, expression of ADH4 in African-American FFPE samples makes that sample more likely to be associated with the PCa population. The ADH1B/HNF1B expression profile of these samples also matches the expression profile the PCa population. Early identification of elevated risk for developing prostatic disease may allow limited health care resources to more effectively targeted and allow both physicians and patients to make better clinical decisions.

A Caucasian sample and an Asian sample were identified by the present methods with high ADH4 expression. Expression of ADH4 in urine may also be indicative of an increased PCa risk for a subpopulation of Caucasians. Additionally, one Asian sample with high ADH4 expression was found but the limited number of Asian samples did not allow discernment of increased risk for this sample group.

Ancestry Informative Markers

A panel of 92 single nucleotide polymorphisms (SNPs) listed in Table III was used as ancestry informative markers (AIM) to confirm self-reported racial identity of 21 FFPE samples).

TABLE III
List of 92 SNPs used as Ancestry Informative Markers (AIMs).

| rs# | chromosome | Position | Allele 1 | Allele 2 |
|---|---|---|---|---|
| rs1004704 | 16 | 48315382 | T | C |
| rs10131076 | 14 | 78764426 | A | G |
| rs1013459 | 18 | 11690534 | A | G |
| rs10214949 | 7 | 78660644 | A | G |
| rs1036543 | 2 | 133886983 | G | A |
| rs10486576 | 7 | 27861415 | T | C |
| rs10488172 | 7 | 132751390 | T | G |
| rs10491097 | 17 | 19523240 | C | T |
| rs10491654 | 9 | 97519365 | T | C |
| rs10492585 | 13 | 103084177 | A | G |
| rs10497705 | 2 | 190694557 | C | T |
| rs10498255 | 2 | 231814769 | A | G |
| rs10498919 | 6 | 78943329 | C | G |
| rs10500505 | 16 | 64718164 | A | T |
| rs10501474 | 11 | 80126955 | C | T |
| rs10506816 | 12 | 78427325 | A | T |
| rs10507688 | 13 | 61204229 | G | A |
| rs10510791 | 3 | 57251433 | C | G |
| rs10515919 | 2 | 75515133 | A | G |
| rs10517518 | 4 | 61798796 | G | A |
| rs10519979 | 4 | 150212578 | A | G |
| rs10520440 | 4 | 181494895 | G | T |
| rs10520678 | 15 | 86667051 | C | T |
| rs1073319 | 2 | 29414989 | G | A |
| rs12953952 | 18 | 65886896 | G | A |
| rs1353251 | 5 | 35902708 | C | T |
| rs138022 | 22 | 38856075 | A | G |
| rs1395771 | 3 | 97784481 | G | A |
| rs1397618 | 10 | 120497262 | T | A |
| rs1398829 | 4 | 21774158 | A | T |
| rs1451928 | 14 | 46330779 | A | C |
| rs1470524 | 2 | 45104050 | G | A |
| rs1498991 | 3 | 20875097 | G | C |
| rs1517634 | 2 | 224386024 | A | G |
| rs153898 | 5 | 94262695 | T | C |
| rs1898280 | 8 | 116031043 | C | T |
| rs1919550 | 3 | 122685074 | T | A |
| rs1934393 | 1 | 48578535 | C | G |
| rs1990745 | 5 | 103458138 | C | T |
| rs2035573 | 3 | 132534415 | T | C |
| rs2042762 | 18 | 33529609 | C | T |
| rs2208139 | 20 | 38594383 | T | C |
| rs2253624 | 17 | 70329204 | G | T |
| rs2296274 | 14 | 59907219 | A | G |
| rs249847 | 12 | 97370184 | G | A |
| rs2569029 | 5 | 155221405 | G | T |
| rs2585901 | 13 | 19218271 | G | A |
| rs2592888 | 1 | 156802365 | T | C |
| rs2785279 | 10 | 33713882 | C | T |
| rs2817611 | 1 | 11322709 | C | T |
| rs2829454 | 21 | 25194942 | G | A |
| rs2840290 | 9 | 16723957 | A | G |
| rs30125 | 16 | 14321100 | G | A |
| rs304051 | 3 | 4553306 | C | T |
| rs3768176 | 1 | 56937923 | G | C |
| rs3806218 | 1 | 144518860 | C | T |
| rs3828121 | 1 | 81844793 | A | G |
| rs3860446 | 2 | 104110751 | T | C |
| rs4013967 | 9 | 72354189 | G | A |
| rs4034627 | 12 | 126750571 | A | G |
| rs4076700 | 12 | 115795273 | A | G |
| rs4130405 | 8 | 99377358 | C | A |
| rs4130513 | 16 | 78238277 | A | G |
| rs4625554 | 12 | 4286565 | G | A |
| rs4657449 | 1 | 162652658 | A | G |
| rs4733652 | 8 | 129801116 | C | T |
| rs4852696 | 2 | 83126181 | C | G |
| rs4934436 | 10 | 90447897 | T | C |
| rs5000507 | 13 | 79886955 | A | T |
| rs567992 | 11 | 105800114 | C | T |
| rs6569792 | 6 | 132675321 | A | G |
| rs6684063 | 1 | 30201812 | T | G |
| rs6804094 | 3 | 188378883 | A | T |
| rs6883095 | 5 | 79975120 | G | A |
| rs6911727 | 6 | 9061397 | T | C |
| rs708915 | 20 | 8395667 | A | T |
| rs719776 | 4 | 33583840 | C | G |
| rs7535375 | 1 | 232954308 | C | T |
| rs798887 | 19 | 59485000 | A | G |
| rs802524 | 7 | 145343383 | T | C |
| rs842634 | 2 | 61065756 | C | T |
| rs868179 | 2 | 177752041 | T | C |
| rs879780 | 11 | 129545756 | C | T |
| rs888861 | 19 | 40073692 | T | C |
| rs9292118 | 5 | 55916081 | C | T |
| rs9295316 | 6 | 158476352 | T | A |
| rs9302185 | 15 | 52670920 | C | T |
| rs9307613 | 4 | 130816225 | T | A |
| rs9310888 | 3 | 29261727 | G | A |
| rs9323178 | 14 | 21103774 | A | G |
| rs9325872 | 8 | 20490544 | A | G |
| rs993314 | 6 | 73434170 | C | T |

10 ng of genomic DNA isolated as described from 80 μm of FFPE tissue was used in a four-plex PCR reaction using 10×PCR buffer, 3.0 μM MgCl₂, 200 μM of each dNTP, 0.2 μM of each of four forward and reverse primers, and 1.25 U HotStart DNA polymerase in 30 μl reactions. Reactions were incubated at 94° C. for 15 minutes followed by 45 cycles of 94° C. for 30 seconds, 60° C. for 30 seconds, and 72° C. for 30 seconds. Following the cycles, the samples were incubated at 72° C. for an additional 5 minutes and then held at 4° C. One of each PCR primer pair was biotinylated which converted the PCR products to single-stranded DNA templates prior to pyrosequencing. The 30 μl reactions were divided into two and used for duplicate pryosequencing reactions. Allele quantification of each SNP was done using QCpG software (Qiagen, Valencia Calif.).

FIG. 10 is a graph showing the inferred proportion of African Ancestry in 21 individuals resulting from the analysis of 92 AIM markers (SNPs). Samples 1-12 are from individuals who self-identified as being Black and samples 13-21 are from individuals who self-identified as being White.

A panel of 92 single nucleotide polymorphisms (SNPs), listed in Table III, was used as ancestry informative markers (AIM) to confirm self-reported racial identity of 21 FFPE samples. Sequenced AIMs (SNPs) were analyzed for genetic admixture using the Bayesian cluster algorithm STRUCTURE described in Pritchard et al., Genetics, 155: 945-959, 2000. Of the 21 FFPE samples analyzed, 12 originated from individuals who self-identified as Black (African descent) and 9 originated from individuals who self-identified as White (European descent). Results from the STRUCTURE analysis divided the 21 samples into 2 clusters that mirrored the membership of the 2 self-identification groups. The first cluster, samples 1-12 in FIG. 10, contains all 12 samples from the self-identified Black donors and the second cluster, samples 13-21 in FIG. 10, contains all 9 samples from the self-identified White donors. The mean admixture proportions for the first cluster are 0.864 African and 0.136 European. For the second cluster, the mean admixture proportions are 0.953 European and 0.047 African. The net nucleotide distance (allelic frequency divergence) between the 2 clusters is 0.168.

Additional information regarding AIM listed in Table III is found in Table IV along with additional AIM that may be used.

TABLE IV

| | | | | | δ values | | |
|---|---|---|---|---|---|---|---|
| | | Allele frequencies | | | | African- | European- |
| | | | | Native | African- | Native | Native |
| AIM rs no. | Chromosome | African | European | American | European | American | American |
| rs1004704 | 16 | 0.028 | 0.214 | 0.867 | 0.187 | 0.839 | 0.652 |
| rs10131076 | 14 | 0.694 | 0.071 | 0.000 | 0.623 | 0.694 | 0.071 |
| rs1013459 | 18 | 0.233 | 0.900 | 0.967 | 0.667 | 0.733 | 0.067 |
| rs10214949 | 7 | 0.281 | 0.845 | 0.967 | 0.564 | 0.685 | 0.121 |
| rs10248051 | 7 | 0.083 | 0.833 | 0.200 | 0.750 | 0.117 | 0.633 |
| rs1036543 | 2 | 0.750 | 0.024 | 0.767 | 0.726 | 0.017 | 0.743 |
| rs10484578 | 6 | 0.944 | 0.375 | 0.067 | 0.569 | 0.878 | 0.308 |
| rs10486576 | 7 | 0.944 | 0.900 | 0.200 | 0.044 | 0.744 | 0.700 |
| rs10488172 | 7 | 0.972 | 0.786 | 0.200 | 0.187 | 0.772 | 0.586 |
| rs10491097 | 17 | 0.056 | 0.647 | 0.133 | 0.592 | 0.078 | 0.514 |
| rs10491654 | 9 | 0.500 | 0.286 | 0.900 | 0.214 | 0.400 | 0.614 |
| rs10492585 | 13 | 0.100 | 0.940 | 1.000 | 0.840 | 0.900 | 0.060 |
| rs10497705 | 2 | 0.118 | 0.238 | 0.867 | 0.120 | 0.749 | 0.629 |
| rs10498255 | 2 | 0.250 | 0.810 | 0.967 | 0.560 | 0.717 | 0.157 |
| rs10498919 | 6 | 0.000 | 0.000 | 0.733 | 0.000 | 0.733 | 0.733 |
| rs10500505 | 16 | 0.056 | 0.214 | 0.800 | 0.159 | 0.744 | 0.586 |
| rs10501474 | 11 | 0.056 | 0.643 | 0.800 | 0.587 | 0.744 | 0.157 |
| rs10506816 | 12 | 0.861 | 0.000 | 0.100 | 0.861 | 0.761 | 0.100 |
| rs10507688 | 13 | 0.000 | 0.167 | 0.733 | 0.167 | 0.733 | 0.567 |
| rs10508349 | 10 | 0.056 | 0.012 | 0.767 | 0.044 | 0.711 | 0.755 |
| rs10510791 | 3 | 0.972 | 0.488 | 0.133 | 0.484 | 0.839 | 0.354 |
| rs10515535 | 5 | 1.000 | 0.286 | 0.133 | 0.714 | 0.867 | 0.152 |
| rs10515919 | 2 | 0.861 | 0.881 | 0.167 | 0.020 | 0.694 | 0.714 |
| rs10517518 | 4 | 0.944 | 0.857 | 0.133 | 0.087 | 0.811 | 0.724 |
| rs10519979 | 4 | 0.167 | 0.451 | 0.967 | 0.285 | 0.800 | 0.515 |
| rs10520440 | 4 | 1.000 | 0.786 | 0.167 | 0.214 | 0.833 | 0.619 |
| rs10520678 | 15 | 0.154 | 0.732 | 1.000 | 0.578 | 0.846 | 0.268 |
| rs1073319 | 2 | 0.917 | 0.810 | 0.167 | 0.107 | 0.750 | 0.643 |
| rs12953952 | 18 | 0.139 | 0.927 | 0.967 | 0.788 | 0.828 | 0.040 |
| rs1353251 | 5 | 0.972 | 0.798 | 0.267 | 0.175 | 0.706 | 0.531 |
| rs138022 | 22 | 0.833 | 0.262 | 0.033 | 0.571 | 0.800 | 0.229 |
| rs1395771 | 3 | 0.656 | 0.024 | 0.567 | 0.632 | 0.090 | 0.543 |
| rs1397618 | 10 | 0.294 | 0.952 | 1.000 | 0.658 | 0.706 | 0.048 |
| rs1398829 | 4 | 0.222 | 0.976 | 1.000 | 0.754 | 0.778 | 0.024 |
| rs1451928 | 14 | 0.861 | 0.857 | 0.200 | 0.004 | 0.661 | 0.657 |
| rs1470524 | 2 | 0.222 | 0.786 | 0.533 | 0.563 | 0.311 | 0.252 |
| rs1477277 | 5 | 1.000 | 0.345 | 0.300 | 0.655 | 0.700 | 0.045 |
| rs1498991 | 3 | 0.944 | 0.976 | 0.250 | 0.032 | 0.694 | 0.726 |
| rs1517634 | 2 | 0.833 | 0.833 | 0.000 | 0.000 | 0.833 | 0.833 |
| rs153898 | 5 | 0.083 | 0.738 | 0.033 | 0.655 | 0.050 | 0.705 |
| rs1898280 | 8 | 0.889 | 0.238 | 0.833 | 0.651 | 0.056 | 0.595 |
| rs1919550 | 3 | 0.028 | 0.024 | 0.833 | 0.004 | 0.806 | 0.810 |
| rs1934393 | 1 | 0.222 | 0.842 | 0.300 | 0.620 | 0.078 | 0.542 |
| rs1984473 | 3 | 0.056 | 0.631 | 0.967 | 0.575 | 0.911 | 0.336 |
| rs1990743 | 17 | 0.639 | 0.634 | 0.067 | 0.005 | 0.572 | 0.567 |
| rs1990745 | 5 | 1.000 | 0.881 | 0.233 | 0.119 | 0.767 | 0.648 |
| rs2035573 | 3 | 0.972 | 0.655 | 0.133 | 0.317 | 0.839 | 0.521 |
| rs2042762 | 18 | 0.000 | 0.000 | 0.733 | 0.000 | 0.733 | 0.733 |
| rs2208139 | 20 | 0.833 | 0.667 | 0.033 | 0.167 | 0.800 | 0.633 |
| rs2253624 | 17 | 0.176 | 1.000 | 1.000 | 0.824 | 0.824 | 0.000 |
| rs2296274 | 14 | 0.118 | 0.750 | 0.967 | 0.632 | 0.849 | 0.217 |
| rs249847 | 12 | 0.056 | 0.463 | 0.967 | 0.408 | 0.911 | 0.503 |
| rs2569029 | 5 | 0.889 | 0.667 | 0.133 | 0.222 | 0.756 | 0.533 |
| rs257748 | 5 | 0.806 | 0.381 | 0.967 | 0.425 | 0.161 | 0.586 |
| rs2585901 | 13 | 0.765 | 0.854 | 0.067 | 0.089 | 0.698 | 0.787 |
| rs2592888 | 1 | 0.088 | 0.762 | 1.000 | 0.674 | 0.912 | 0.238 |
| rs2595456 | 11 | 0.306 | 0.524 | 0.000 | 0.218 | 0.306 | 0.524 |
| rs2711070 | 2 | 0.139 | 0.464 | 0.967 | 0.325 | 0.828 | 0.502 |
| rs2785279 | 10 | 0.824 | 0.262 | 0.067 | 0.562 | 0.757 | 0.195 |
| rs2817611 | 1 | 0.278 | 0.952 | 0.967 | 0.675 | 0.689 | 0.014 |
| rs2829454 | 21 | 0.056 | 0.274 | 0.933 | 0.218 | 0.878 | 0.660 |
| rs2840290 | 9 | 0.861 | 0.268 | 0.900 | 0.593 | 0.039 | 0.632 |
| rs30125 | 16 | 0.639 | 0.049 | 0.100 | 0.590 | 0.539 | 0.051 |
| rs304051 | 3 | 0.750 | 0.548 | 0.000 | 0.202 | 0.750 | 0.548 |
| rs354747 | 20 | 0.083 | 0.643 | 0.767 | 0.560 | 0.683 | 0.124 |
| rs3768176 | 1 | 0.972 | 0.929 | 0.233 | 0.044 | 0.739 | 0.695 |
| rs3806218 | 1 | 0.059 | 0.667 | 0.821 | 0.608 | 0.763 | 0.155 |
| rs3828121 | 1 | 1.000 | 0.869 | 0.300 | 0.131 | 0.700 | 0.569 |
| rs3860446 | 2 | 0.972 | 0.357 | 1.000 | 0.615 | 0.028 | 0.643 |
| rs4013967 | 9 | 0.059 | 0.655 | 1.000 | 0.596 | 0.941 | 0.345 |
| rs4034627 | 12 | 0.781 | 0.048 | 0.033 | 0.734 | 0.748 | 0.014 |
| rs4076700 | 12 | 0.206 | 0.833 | 0.900 | 0.627 | 0.694 | 0.067 |
| rs4130405 | 8 | 0.000 | 0.190 | 0.800 | 0.190 | 0.800 | 0.610 |
| rs4130513 | 16 | 0.722 | 0.083 | 0.192 | 0.639 | 0.530 | 0.109 |
| rs4625554 | 12 | 0.167 | 0.298 | 0.867 | 0.131 | 0.700 | 0.569 |
| rs4657449 | 1 | 0.083 | 0.071 | 0.733 | 0.012 | 0.650 | 0.662 |
| rs4733652 | 8 | 0.917 | 0.762 | 0.100 | 0.155 | 0.817 | 0.662 |
| rs4762106 | 12 | 0.806 | 0.095 | 0.667 | 0.710 | 0.139 | 0.571 |
| rs4852696 | 2 | 0.250 | 0.905 | 0.700 | 0.655 | 0.450 | 0.205 |
| rs4934436 | 10 | 0.694 | 0.452 | 0.967 | 0.242 | 0.272 | 0.514 |
| rs5000507 | 13 | 0.139 | 0.713 | 1.000 | 0.574 | 0.861 | 0.288 |
| rs567992 | 11 | 1.000 | 0.845 | 0.333 | 0.155 | 0.667 | 0.512 |
| rs6569792 | 6 | 0.111 | 0.679 | 0.800 | 0.567 | 0.689 | 0.121 |
| rs6684063 | 1 | 0.222 | 0.833 | 0.167 | 0.611 | 0.056 | 0.667 |
| rs6804094 | 3 | 0.972 | 0.654 | 0.1.33 | 0.318 | 0.839 | 0.521 |
| rs6883095 | 5 | 0.861 | 0.548 | 0.033 | 0.313 | 0.828 | 0.514 |
| rs6911727 | 6 | 0.139 | 0.476 | 1.000 | 0.337 | 0.861 | 0.524 |
| rs708915 | 20 | 0.735 | 0.100 | 0.733 | 0.635 | 0.002 | 0.633 |
| rs719776 | 4 | 0.917 | 0.143 | 0.167 | 0.774 | 0.750 | 0.024 |
| rs7463344 | 8 | 0.639 | 0.000 | 0.000 | 0.639 | 0.639 | 0.000 |
| rs7535375 | 1 | 0.111 | 0.714 | 0.800 | 0.603 | 0.689 | 0.086 |
| rs798887 | 19 | 0.778 | 0.854 | 0.133 | 0.076 | 0.644 | 0.720 |
| rs802524 | 7 | 0.278 | 0.929 | 0.967 | 0.651 | 0.689 | 0.038 |
| rs842634 | 2 | 0.972 | 0.738 | 0.233 | 0.234 | 0.739 | 0.505 |
| rs868179 | 2 | 0.700 | 0.073 | 0.000 | 0.627 | 0.700 | 0.073 |
| rs879780 | 11 | 0.667 | 0.048 | 0.000 | 0.619 | 0.667 | 0.048 |
| rs888861 | 19 | 0.000 | 0.738 | 0.900 | 0.738 | 0.900 | 0.162 |
| rs9292118 | 5 | 0.176 | 0.833 | 0.967 | 0.657 | 0.790 | 0.133 |
| rs9295316 | 6 | 0.029 | 0.098 | 0.867 | 0.068 | 0.837 | 0.769 |
| rs9302185 | 15 | 0.875 | 0.190 | 0.033 | 0.685 | 0.842 | 0.157 |
| rs9307613 | 4 | 0.094 | 0.775 | 0.833 | 0.681 | 0.740 | 0.058 |
| rs9310888 | 3 | 0.667 | 0.075 | 0.000 | 0.592 | 0.667 | 0.075 |
| rs9320808 | 6 | 0.861 | 0.095 | 0.967 | 0.766 | 0.106 | 0.871 |
| rs9323178 | 14 | 0.176 | 0.452 | 0.967 | 0.276 | 0.790 | 0.514 |
| rs9325872 | 8 | 0.944 | 0.321 | 1.000 | 0.623 | 0.056 | 0.679 |
| rs948360 | 11 | 0.250 | 0.974 | 1.000 | 0.724 | 0.750 | 0.026 |
| rs993314 | 6 | 0.900 | 0.167 | 0.700 | 0.733 | 0.200 | 0.533 |

Determination of the Genotype of African-American Males Expressing ADH4 and Comparison of that Genotype to Both African-American Males and Caucasian Males not Expressing ADH4

Patients in the sample population of Tables 1 and 2 self-identified themselves as either African-American or Caucasian. Given the broad genetic background of these racial groups, it was unexpected to find expression of ADH4 in the majority of African-American men with prostatic disease while ADH4 expression was not detected in any of the samples from Caucasian men with prostatic disease. To determine if the African-American men that express ADH4 have a common genetic link, ancestry informative markers (AIM) will be determined in these samples and compared to the AIM of the samples from African-American men that do not express ADH4. In addition, a random sampling of the AIM expressed by Caucasian men will also be determined. Genomic DNA will be extracted from samples and run against an Affymetrix Genome-Wide Human SNP Microarray 6.0. Admixture mapping will be completed using HapMap, Structure, and AncestryMap to determine common ancestry markers in each sample group. Comparison of the markers between the groups will be used to determine if the expression of ADH4 results from a common genetic background.

Correlation of Detectable ADH4 Expression in Urine Samples with Detectable ADH4 Expression in Prostate Epithelium.

Samples of urine will be obtained from both self-identified African-Americans and Caucasians with prostatic disease. A sample of prostate tissue will be obtained from formalin-fixed paraffin embedded biopsy materials from the same patients. Total RNA will be extracted from both prostate epithelial cells in the urine sample and prostate epithelium from biopsy materials using the Recover All RNA isolation kit according to manufacturer's protocol (Applied Biosystems). Expression of ADH4 will be determined using quantitative RT-PCR with Taqman Gene Expression Assays (Applied Biosystems) that were validated for ADH4 (Applied Biosystems). Reactions will be run in triplicate and analyzed in a MX30005P thermocycler (Agilent Technologies, Santa Clara Calif.). Commercially validated TAQMAN probes for RPLPO (large ribosomal protein) and HPRT1 (hypoxanthine phosphoribosyltransferase 1) will be used as endogenous controls for relative quantification (Applied Biosystems). Expression of ADH4 in both urine and biopsy prostatic epithelium will be correlated to determine if ADH4 isozyme expression profile is the same in both sample types.

### Example 8

### Determination of ADH1B Expression in a Urine Sample Using Quantitative RT-PCR

A urine sample of at least 30 milliliters is collected from a subject in a plastic RNase-free 100 milliliter collection cup containing 5 milliliters of RNA preservative, AssayAssure (Zymo Research, Irvine, Calif.). All samples are stored at 4° C. until processing. Processing occurs within 24 hours of collection.

Prostate cells are recovered from a urine sample and processed as described in Example 7. Resulting total cellular RNA is used to determine ADH1B gene expression using quantitative RT-PCR as described herein. A PSA Ct of 26 or lower is used as a marker for sufficient recovery of prostate cells.

The resulting determined level of ADH1B expression in the urine sample is used to identify subjects at increased risk of a proliferative disorder of the prostate gland. Decreased expression of the alcohol dehydrogenase 1B compared to a control is indicative of a proliferative disorder of the prostate gland in the subject.

Sequences

Amino acid Sequence of human ADH1B;
SEQ ID NO: 37
MSTAGKVIKCKAAVLWEVKKPFSIEDVEVAPPKAYEVRIKMVAVGICHTDDHVVSGNLVTPLPVILGHEAAGIVESV
GEGVTTVKPGDKVIPLFTPQCGKCRVCKNPESNYCLKNDLGNPRGTLQDGTRRFTCRGKPIHHFLGTSTFSQYTVVD
ENAVAKIDAASPLEKVCLIGCGFSTGYGSAVNVAKVTPGSTCAVFGLGGVGLSAVMGCKAAGAARIIAVDINKDKFA
KAKELGATECINPQDYKKPIQEVLKEMTDGGVDESFEVIGRLDTMMASLLCCHEACGTSVIVGVPPASQNLSINPML
LLTGRTWKGAVYGGEKSKEGIPKLVADFMAKKESLDALITHVLPFEKINEGFDLLHSGKSIRTVLTF
mRNA encoding human ADH1B;
SEQ ID NO: 38
aatatatctgctttatgcactcaagcagagaagaaatccacaaagactcacagtctgctggtgggcagagaagacag
aaacgacatgagcacagcaggaaaagtaatcaaatgcaaagcagctgtgctatgggaggtaaagaaaccgttttcca
ttgaggatgtggaggttgcacctcctaaggcttatgaagttcgcattaagatggtggctgtaggaatctgtcacaca
gatgaccacqtggttagtggcaacctggtgaccccgcttcctgtgattttaggccatgaggcagccggcatcgtgga
gagtgttggagaaggggtqactacagtcaaaccaggtgataaagtcatcccgctctttactcgtcagtgtggaaaat
gcagagtttgtaaaaaccgggagagcaactactgcttgaaaaatgatctaggcaatgctcgggggaccctgcaggat
ggcaccaggaggttcacctgcagggggaagcccattcaccacttccttggcaccagcaccttctcccagtacacggt
ggtggatgagaatggagtggccaaaattgatgcagcctcgcgcctggagaaagtctgcctcattggctgtggattct
cgactggttatgggtctgcagttaacgttgccaaggtcaccccaggctctacctgtgctgtgtttggcctgggaggg
gtcggcctatctggtgttatgggctgtaaagcagctggagcagccagaatcattgcggtggacatcaacaaggacaa
atttgcaaaggccaaagagttgggtgccactgaatgcatcaaccctcaagactacaagaaacccattcaggaagtgc
taaaggaaatgactgatggaggtgtggatttttcgtttgaagtcatcggtgggcttgacaccatgatggcttccctg
ttatgttgtcatgaggcatgtggcacaagcgtcatcgtaggggtacctcgtgcttcccagaacctctcaataaaccc
tatggtggtactgactggacgcacctggaagggggctgtttatggtggctttaagagtaaagaaggtatcccaaaac
ttgtggctgattttatggctaagaagttttgactggatgcgttaataacccatgttttaccttttgaaaaaataaat
gaaggatttgacctggttcactctgggaaaagtatccgtaccgtcctgacgttttgaggcaatagagatgccttccc
ctgtagcagtgttcagccucctctaccctacaagatctggagcaacagctaggaaatatcattaattcagctcttca
gagatgttatcaataaattacacatgggggctttccaaagaaatggaaattgatgggaaattatttttcaggaaaat
ttaaaattcaagtgagaagtaaataaagtgttgaacatcagctggggaattgaagccaacaaaccttccttcrtaac
cattctactgtgtcacctttgccattgaggaaaaatattcctgtgacttcttgcatttttggtatcttcataatctt
tagtcatcgaatcccagtggaggggacccttttacttgccctgaacatacacatgctgggccattgtgattgaagtc
ttctaactctgtctcagttttcactgtcgacattttcctttttctaataaaaatgtaccaaatccctggggtaaaag
ctagggtaaggtaaaggatagactcacatttacaagtagtgaaggtccaagagttctaaatacaggaaatttcttag
gaactcaaataaaatgccccacattttactacagtaaatggcagtgtttttatgacttttatactatttctttatgg
tcgatatacaattgattttztaaaataataggagatttcttggttcatatgacaaagcctcaattactaattgtaaa
aactgaactattcccagaatcatgttcaaaaaatctgtaatttttgctgatgaaagtgcttcattgactaaacagta
ttagtttgtggctataaatgattatttagatgatgactgaaaatgtgtataaagtaattaaaagtaatatggtggct
ttaagtgtagagatgggatggcaaatgctgtgaatgcagaatgtaaaattggtaactaagaaatggcacaaacacct
taagcaatatattttggtagtagatatatatatacacatacatatatacacatatacaaatgtatatttttgcaaaa
ttgttztcaatctagaacttttctattaactacgatgtcttaaaatcaagtctataatggtagcattagtttaatat
tttgaatatgtaaagacctgtgttaatggtttgttaatgcttttcccactctcatttgttaatgctttcccactctc
aggggaaggatttgcattttgagctttatctctaaatgtgacatgcaaagattattcctggtaaaggaggtagctgt
ctccaaaaatgctattgttgcaatatctacattctatttcatattatgaaagaccttagacataaagtaaaatagtt
tatcatttactgtgtgatcttcagtaagtctctcaggctctctgagcttgttcatcctttgttttgaaaaaattact
caaccaatccattacagcttaaccaagattaaatgggatgatgttaatgaaagagcttcgccaaaaaaaaa
Amino acid Sequence of human ADH1C;
SEQ ID NO: 39
MSTAGKVIKCKAAVLWELKKPFSIEEVEVAPPKAHEVRIKMVAAGICRSDEGVVSGNLNTPLPVILGHEAAGIVESV
GEGVTTVKPGDKVIPLETPQCGKCRICKNPESNYCIKNDLGNPRGTLQDGTRRETCSGKPIHHEVGVSTESQYTVVD
ENAVAKIDAASPLEKVCLIGCGFSTGYGSAVKVAKVTPGSTCAVFGLGGVGLSVVMGCKAAGAARIIAVDINKDKFA
KAKELGATECINPQDYKKPIQEVLKEMTDGGVDFSFEVIGRLDTMMASLLCCHEACGTSVIVGVPPDSQNLSINPML
LLTGRTWKGAIFGGEKSKESVPKLVADFMAKKESLDALITNILPFEKINEGFDLLRSGKSIRTVLTF
mRNA encoding human ADH1C;
SEQ ID NO: 40
aacatacctgctttatgcactcaagcagagaagaaatccacaagtactcaccagcctcctggtctgcagagaagaca
gaatcaatatgagcacagcaggaaaagtaatcaaatgcaaagcagctgtgctatgggagttaaagaaacccttttcc
attgaggaggtagaggttgcacctcctaaggctcatgaagttcgcattaagatggtggctgcaggaatctgtcgttc
agatgagcatgtggttagtggcaacctggtgaccccccttcctgtgattttaggccatgaggcagccggcatcgtgg
aaagtgttggagaaggggtgactacagtcaaaccaggtgataaagtcatcccgctctttactcctcagtgtggaaaa
tgcagaatttgtaaaaacccagaaagcaactactgcttgaaaaatgatctaggcaatcctcgggggaccctgcagga
tggcaccaggaggttcacctgcagcgggaagcccatccaccacttcgtcggcgtcagcaccttctcccagtacacag
tggtggatgagaatgcagtggccaaaattgatgcagcctcgcccctggagaaagtctgcctcattggctgtggattt
tcgactggttatgggtctgcagtcaaagttgccaaggtcaccccagggtctacctgtgctgtgtttggcctgggagg
ggteggcctatctgttgttatgggctgtaaagcagctggagcagccagaatcattgctgtggacatcaacaaggaca
aatttgcaaaggctaaagagttgggtgccactgaatgcatcaaccctcaagactacaagaaacccattcaggaagtg
ctaaaggaaatgactgatggaggtgtggatttttcgtttgaagtcatcggtcggcttgacaccatgatggcttccct
gttatgttgtcatgaggcatgtggcacaagtgtcattgtaggggtacctcctgattcccagaacctctcaataaacc
ctatgctgctactgactggacgcacgtggaaaggagctatttttggaggctttaagagtaaagaatctgcccccaaa
cttgtggctgactttatggctaagaagttttcactggatgcattaataacaaatattttaccttttgaaaaaataaa
tgaaggatttgacctgcttcgctctggaaagagtatccgtaccgtcctgacgttttgaaacaatacagatgccttcc
cttgtagcagttttcagcctcatctaccctacatgatctggagcaacagctaggaaatatcattaattctgctcttc
agagatgttaaaaataaattacacgtgggagctttccaaagaaatggaaattgatgggaaattatttgtcaagcaaa
tgtttaaaatccaaatgagaactaaataaagtgttgaacatcaactggggaattgaagccaataaaccttccttctt
aaccattcaaaaaaaaaaaaaaaaaaaaaaaaaa
Amino acid Sequence for human ADH4, variant 1;
SEQ ID NO: 41
MKCLVSRHTVRETLDMVFQRMSVEAGVIKCKAAVLWEQKQPFSIEEIEVAPPKTKEVRIKILATGICRTDDHVIKGT
MVSKFPVIVGHEATGIVESIGEGVTTVKPGDKVIPLFLPQCRECNACRNPDGNLCIRSDITGRGVLADGTTRFTCKG
KPVHHFMNTSTFTEYTVVDESSVAKIDDAAPPEKVCLIGCGFSTGYGAAVKTGKVKPGSTCVVFGLGGVGLSVIMGC
KSAGASRIIGIDLNKDKFEKAMAVGATECISPKDSTKPISEVLSEMTGNNVGYTFEVIGHLETMIDALASCHMNYGT
SVVVGVPPSAKMLTYDPMLLFTGRTWKGCVFGGLKSRDDVPKLVTEFLAKKFDLDQLITHVLPFKKISEGFELLNSG
QSIRTVLTF
Amino acid Sequence for human ADH4, variant 2;
SEQ ID NO: 42
MFAEIQIQDKDRMGTAGKVIKCKAAVLWEQKQPFSIEEIEVAPPKTKEVRIKILATGICRTDDH
VIKGTMVSKFPVIVGHEATGIVESIGEGVTTVKPGDKVIPLFLPQCRECNACRNPDGNLCIRSD
ITGRGVLADGTTRFTCKGKPVHHFMNTSTFTEYTVVDESSVAKIDDAAPPEKVCLIGCGFSTGY
GAAVKTGKVKPGSTCVVFGLGGVGLSVIMGCKSAGASRIIGIDLNKDKFEKAMAVGATECISPK
DSTKPISEVLSEMTGNNVGYTFEVIGHLETMIDALASCHMNYGTSVVVGVPPSAKMLTYDPMLL
FTGRTWKGCVFGGLKSRDDVPKLVTEFLAKKFDLDQLITHVLpFKKISEGFELLNSGQSIRTVL
TF
mRNA encoding human ADH4, variant 1;
SEQ ID NO: 43
aattaaacacttggagatattccttgaggaatgaaatgcttggtgagcaggcatacagtgagggaaacactggatat
ggtgtttcagagaatgtcagtggaagcaggggttattaaatgcaaagcagctgtgctttgggagcagaagcaaccct
tctccattgaggaaatagaagttgccccaccaaagactaaagaagttcgcattaagattttggccacaggaatatgt
cgcacagatgaccatgtgataaaaggaacaatggtgtccaagtttccagtgattgtgggacatgaggcaactgggat
tgtagagagcattggagaaggagtgactacagtgaaaccaggtgacaaagtcatccctctctttctgccacaatgta
gagaatgcaatgcttgtcgcaacccagatggcaacctttgcattaggagcgatattactggtcgtggagtactggct
gatggcaccaccagatttacatgcaagggcaaaccagtccaccacttcatgaacaccagtacatttaccgagtacac
agtggtggatgaatcttctgttgctaagattgatgatgcagctcctcctgagaaagtccgtttaattggctgtgggt
tttccactggatatggcgctgatgttaaaactggcaaggtcaaacctggttccacttgcgtcgtctttggcctggga
ggagttggcctgtcagtcatcatgggctgtaagtcagctggtgcatctaggatcattgggattgacctcaacaaaga
caaatttgagaaggccatggctgtaggtgccactgagtgtatcagtcccaaggactctaccaaacccatcagtgagg
tgctgtcagaaatgacaggcaacaacgtgggatacacctttgaagttattgggcatcttgaaaccatgattgatgcc
ctggcatcctgccacatgaactatgggaccagcgtggttgtaggagttcctccatcagccaagatgctcacctatga
cccgatgttgatcttcactggacgcacatggaagggatgtgtctttggaggtttgaaaagcagagatgatgtcccaa
aactagtgactgagttcctggcaaagaaatttgacctggaccagttgataactcatgttttaccatttaaaaaaatc
agtgaaggatttgagctgctcaattcaggacaaagcattcgaacggtcctgacgttttgagatccaaagtggcagga
ggtctgtgttgtcatggtgaactggagtttctcttgtgagagttccctcatctgaaatcatgtatctgtctcacaaa
tacaagcataagtagaagatttgttgaagacatagaaccattataaagaattattaacctttataaacatttaaagt
cttgtgagcacctgggaattagtataataacaatgttaatatttttgatttacattttgtaaggctataattgtatc
ttttaagaaaacatacacttggatttctatgttgaaatggagatttttaagagttttaaccagctgctgcagatata
taactcaaaacagatatagcgtataaagatatagtaaatgcatctcctagagtaatattcacttaacacattgaaac
tattattttttagatttgaatataaatgtattttttaaacacttgttatgagttaagttggattacattttgaaatc
agttcattccatgatgcatattactggattagattaagaaagacagaaaagattaagggacgggcacatttttcaac
gattaagaatcatcattacataacttggtgaaactgaaaaagtatatcatatgggtacacaaggctatttgccagca
tatattaatattttagaaaatattccttttgtaatactgaatataaacatagagctagaatcatattatcatactta
tcataatgttcaatttgatacagtagaattgcaagtccctaagtccctattcactgtgcttagtagtgactccattt
aataaaaagtgtttttagtttttaacaactacactgatgtatctatatatatctataacatgttaaaaattcttaag
aaaattaaaaattatataaaatgaaaaaaaaaaaaaaaaaa
mRNA encoding human ADH4, variant 2;
SEQ ID NO: 44
tttcaatagctttgattatcttgtttttgttagatccctcctcttggtttgatcatagtagttactgtatttctttt
tataagttggtctgcaaagggtagggcttgcagaccattgcaaagttgtgacggctgtgagtcatattgctgaaggt
ggaactctgaagccagactatctatgtgaaggcacaagctgctgttatatacaacagagtgaactgagcatcagtca
gaaaaagtctatgtttgcagaaatacagatccaagacaaagacaggatgggcactgctggaaaagttattaaatgca
aagcagctgtgctttgggagcagaagcaacccttctccattgaggaaatagaagttgccccaccaaagactaaagaa
gttcgcattaagattttggccacaggaatctgtcgcacagatgaccatgtgataaaaggaacaatggtgtccaagtt
tccagtgattgtgggacatgaggcaactgggattgtagagagcattggagaaggagtgactacagtgaaaccaggtg
acaaagtcatccctctctttctgccacaatgtagagaatgcaatgcttgtcgcaacccagatggcaacctttgcatt
aggagcgatattactggtcgtggagtactggctgatggcaccaccagatttacatgcaagggcaaaccagtccacca
cttcatgaacaccagtacatttaccgagtacacagtggtggatgaatcttctgttgctaagattgatgatgcagctc
ctcctgagaaagtctgtttaattggctgtgggttttccactggatatggcgctgctgttaaaactggcaaggtcaaa
cctggttccacttgcgtcgtctttggcctgggaggagttggcctgtcagtcatcatgggctgtaagtcagctggtgc
atctaggatcattgggattgacctcaacaaagacaaatttgagaaggccatggctgtaggtgccactgagtgtatca
gtcccaaggactctaccaaacccatcagtgaggtgctgtcagaaatgacaggcaacaacgtgggatacacctttgaa
gttattgggcatcttgaaaccatgattgatgccctggcatcctgccacatgaactatgggaccagcgtggttgtagg
agttcctccatcagccaagatgctcacctatgacccgatgttgctcttcactggacgcacatggaagggatgtgtct
ttggaggtttgaaaagcagagatgatgtcccaaaactagtgactgagttcctggcaaagaaatttgacctggaccag
ttgataactcatgttttaccatttaaaaaaatcagtgaaggatttgagctgctcaattcaggacaaagcattcgaac
ggtcctgacgttttgagatccaaagtggcaggaggtctgtgttgtcatggtgaactggagtttctcttgtgagagtt
ccctcatctgaaatcatgtatctgtctcacaaatacaagcataagtagaagatttgttgaagacatagaacccttat
aaagaattattaacctttataaacatttaaagtcttgtgagcacctgggaattagtataataacaatgttaatattt
ttgatttacattttgtaaggctataattgtatcttttaagaaaacatacacttggatttctatgttgaaatggagat
ttttaagagttttaaccagctgctgcagatatataactcaaaacagatatagcgtataaagatatagtaaatgcatc
tcctagagtaatattcacttaacacattgaaactattattttttagatttgaatataaatgtattttttaaacactt
gttatgagttaagttggattacattttgaaatcagttcattccatgatgcatattactggattagattaagaaagac
agaaaagattaagggacgggcacatttttcaacgattaagaatcatcattacataacttggtgaaactgaaaaagta
tatcatatgggtacacaaggctatttgccagcatatattaatattttagaaaatattccttttgtaatactgaatat
aaacatagagctagaatcatattatcatacttatcataatgttcaatttgatacagtagaattgcaagtccctaagt
ccctattcactgtgcttagtagtgactccatttaataaaaagtgtttttagtttttaacaactacactgatgtatct
atatatatctataacatgttaaaaattcttaagaaaattaaaaattatataaaatgaaaaaaaaaaaaaaaaaa
Amino acid sequence of human hepatocyte nuclear factor 1B;
SEQ ID NO: 45
MVSKLTSLQQELLSALLSSGVTKEVLVQALEELLPSPNFGVKLETLPLSPGSGAEPDTKPVEHTLTNGHAKGRLSGD
EGSEDGDDYDTPPILKELQALNTEEAAEQRAEVDRMLSEDPWRAAKMIKGYMQQHNIPQREVVDVTGLNQSHLSQHL
NKGTPMKTQKRAALYTWYVRKQREILRQFNQTVQSSGNMTDKSSQDQLLFLEPEFSQQSHGPGQSDDACSEPTNKKM
RRNRFKWGPASQQILYQAYDRQKNPSKEEREALVEECNRAECLQRGVSPSKAHGLGSNINTEVRVYNWFANRRKEEA
FRQKLAMDAYSSNQTHSLNPLLSHGSPHHQPSSSPPNKISGVRYSQQGNNEITSSSTISHHGNSAMVTSQSVLQQVS
PASIDPGHNLLSPDGKMISVSGGGLPPVSTLTNIHSLSHHNPQQSQNLIMTPLSGVMATAQSLNTSQAQSVPVINSV
AGSLAALQPVQFSQQLHSPHQQPLMQSPGSHMAQQPFMAAVTQLQNSHNIYAHKQEPPQYSHTSRFPSAMVVTDTSS
ISTLTNMSSSKOCPLQAW
mRNA encoding human hepatocyte nuclear factor 1B;
SEQ ID NO: 46
aatttgcatatcttatatggcctaatggtggcgatcatggcaagttagaagttttctgactcctttcggaggagcct
ccgggaccccggggagtaacaggtgtctggaggctgaagggtggaggggttcctggatttggggtttgcttgtgaaa
ctcccctccaccctcctctatcgcacccacccaccgcctcacccccttctttttccgtccttggaaaatggtgtcca
agctcacgtcgctccaggaagaactcctgagcgccctgctgagctccggggtcaccaaggaggtgctggttcaggcc
ttggaggagttgctgccatccccgaacttcggggtgaagctggagacgctgcccctgtcccctggcagcggggccga
gcccgacaccaagccggtcttccatactctcaccaagggccacgccaagggccgcttgtccggcgacgagggctccg
aggacggcgacgactatgacacacctcccatcctcaaggagctgcaggcgctcaacaccgaggaggcggcggagcag
cgggcggaggtggaccggatgatcagtgaggacccttggagggctgctaaaatgatcaagggttacatgcagcaaca
caacatccoccagagggaggtggtcgatgtcaccggcctgaaccagtcgcacctctcccagcatctcaacaagggca
cccctatgaagacccagaagcgtgccgctctgtacacctggtacgtcagaaaggaacgagagatcctccgacaattc
aaccagacagtccagagttctggaaatatgacagacaaaaggagtcaggatcagctgctgtttctctttccagagtt
cagtcaacagagccatgggcctgggcagtccgatgatgcctgctctgagcccaccaacaagaagatgcgccgcaacc
ggttcaaatgggggcccgcgtcccagcaaatcttgtaccaggcctacgatcggcaaaagaaccccagcaaggaagag
agagaggccttagtggaggaatgcaacagggcagaatgtttgcagcgaggggtgtccccctccaaagcccacggcct
gggctccaacttggtcactgaggtccgtgtctacaactggtttgcaaaccgcaggaaggaggaggcattccggcaaa
agctggccatggacgcctatagctccaaccagactcacagcctgaaccctctgctctcccacggctccccccaccac
cagcccagctcctctcctccaaacaagctgtcaggagtgcgctacagccaggagggaaacaatgagatcacttcctc
ctcaacaatcagtcaccatggcaacagcgccatggtgaccagccagtcggttttacaggaagtctccccagccagcc
tggacccaggccacaatctcctctcacctgatggtaaaatgatctcagtctcaggaggaggtttgcccccagtcagc
accttgacgaatatccacagcctctcccaccataatccccagcaatctcaaaacctcatcatgacacccctctctgg
agtcatggcaattgcacaaagcctcaacacctcccaagcacagagtgtccctgtcatcaacagtgtggccggcagcc
tggcagccctgcagcccgtccagttctcccagcagctgcacagccctcaccagcagcccctcatgcagcagagccca
ggcagccacatggcccagcagcccttcatggcagctgtgactcagctgcagaactcacacatgtacgcacacaagca
ggaacccccccagtattcccacacctcccggtttccatctgcaatggtggtcacagataccagcagcatcagtacac
tcaccaacatgtcttcaagtaaacagtgtcctctacaagcctggtgatgcccacacaccacttacttcgtgcgcaac
aacaaggaccctgttttccacaccatcaccctctgggcagctgtcatggaaaagcccagtgacctgaccggcacctg
cgagaggtccctgcttacctgacggacgtcctgctggcacctcagacaatccactctcaggaggcgcagcccgaagg
ccagtttccattctatgcagtattgccacaatgcctctcccacgatgtcaaggactcctgtctgtcctggaggtggg
agacaaggaaccaccgaagaggaagcaagaaagccgtactgtctatgttgtgatccttcatcgaacaaactgatgcg
aaaacttgaatctgttactgaaatgaggagagaaggacatgtgctattgaactgagccaaacacactgtaaatatcc
acagactccctcccctgcccccatcccacatgatcttgagatttcttttaaagaagtaaatttgtccaatggctgta
aactataaactactgtaattaagtgcaatttcccctctgtgtcctctcccctctgccctgtatataatactaaagtg
tctattagttttctttgtaaaggtcagagtcaaaatttcaaaagtgatctgtcccctctcccctcatggagaaacat
cctaagtgggaagtgaagccccttgtcctctcccgcgggcctggacacttatggggacagcataccttggactgact
accagctaactccagtctcctgacattaagacacacctctggatccctggaggggctgaatgtagtgtgtcagagta
acatgccagcttcctgtgggccaggagctcagccgtgcactccctaagaaaccccagggcagggaaactggctgttt
gatagcagaagaaaaagttgcagtctcagaaagccttccattaaaacaatttattttatcactaaaaaaa

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method for aiding in detection, assessment and treatment of a proliferative disorder of the prostate gland in a subject, comprising:
assaying a first biological sample comprising prostate gland cells obtained from the subject for expression of one or more biomarkers selected from the group consisting of: alcohol dehydrogenase 1B, alcohol dehydrogenase 1C, alcohol dehydrogenase 4 and hepatocyte nuclear factor 1B; and
determining, based on the expression of the one or more biomarkers in the sample that the subject has, or is at risk of having, a proliferative disorder of the prostate gland, wherein a decrease in alcohol dehydrogenase 1B expression compared to a standard is indicative of hyperplastic disease or neoplastic disease; wherein a decrease in alcohol dehydrogenase 1C expression compared to a standard is indicative of hyperplastic disease or neoplastic disease; wherein detectable expression of alcohol dehydrogenase 4 compared to a standard is indicative of a proliferative disorder of the prostate gland where the subject self-identifies as being of African descent, self-identifies as African-American and/or expresses one or more Ancestry Informative markers indicative of African descent; and wherein an increase in hepatocyte nuclear factor 1B expression compared to a standard is indicative of hyperplastic disease or neoplastic disease.

2. The method of claim 1, further comprising:
modifying or initiating an anti-cancer prostate gland treatment for the subject based on determining that the subject has, or is at risk of having, a proliferative disorder of the prostate gland.

3. The method of claim 1, further comprising:
treating the subject based on determining that the subject has, or is at risk of having, a proliferative disorder of the prostate gland.

4. The method of claim 1, further comprising:
monitoring the subject for development or progression of a proliferative disorder of the prostate gland based on determining that the subject has, or is at risk of having, a proliferative disorder of the prostate gland.

5. The method of claim 1, wherein the subject is human.

6. The method of claim 1, wherein the assaying comprises detection of a biomarker selected from the group consisting of alcohol dehydrogenase 1B, alcohol dehydrogenase 1C, alcohol dehydrogenase 4 and hepatocyte nuclear factor 1B, a nucleic acid encoding a biomarker selected from the group consisting of: alcohol dehydrogenase 1B, alcohol dehydrogenase 1C, alcohol dehydrogenase 4 and hepatocyte nuclear factor 1B and/or function of a biomarker selected from the group consisting of: alcohol dehydrogenase 1B, alcohol dehydrogenase 1C, alcohol dehydrogenase 4 and hepatocyte nuclear factor 1B.

7. The method of claim 3, wherein the monitoring comprises:
assaying a second biological sample comprising prostate gland cells, the second biological sample obtained from the subject at a later time than the biological sample, for expression of one or more biomarkers selected from the group consisting of: alcohol dehydrogenase 1B, alcohol dehydrogenase 1C, alcohol dehydrogenase 4 and hepatocyte nuclear factor 1B; and
determining, based on the expression of the one or more biomarkers in the sample that the subject has, or is at risk of having, a proliferative disorder of the prostate gland, wherein a decrease in alcohol dehydrogenase 1B expression in the second sample compared to the first sample is indicative of progression of a proliferative disorder of the prostate gland and an increase in alcohol dehydrogenase 1B expression in the second sample compared to the first sample is indicative of amelioration of a proliferative disorder of the prostate gland; wherein a decrease in alcohol dehydrogenase 1C expression in the second sample compared to the first sample is indicative of progression of a proliferative disorder of the prostate gland and an increase in alcohol dehydrogenase 1C expression in the second sample compared to the first sample is indicative of amelioration of a proliferative disorder of the prostate gland; wherein an increase in alcohol dehydrogenase 4 expression in the second sample compared to the first sample is indicative of progression of a proliferative disorder of the prostate gland and a decrease in alcohol dehydrogenase 4 expression in the second sample compared to the first sample is indicative of amelioration of a proliferative disorder of the prostate gland; and wherein an increase in hepatocyte nuclear factor 1B expression in the second sample compared to the first sample is indicative of progression of a proliferative disorder of the prostate gland and a decrease in hepatocyte nuclear factor 1B expression in the second sample compared to the first sample is indicative of amelioration of a proliferative disorder of the prostate gland.

8. The method of claim 7, further comprising:
selecting a treatment for the subject based on determining that the expression of one or more biomarkers selected from the group consisting of: alcohol dehydrogenase 1B, alcohol dehydrogenase 1C, alcohol dehydrogenase 4 and hepatocyte nuclear factor 1B, is increased or decreased in the second sample compared to the first sample.

9. The method of claim 7, further comprising:
treating the subject based on determining that the expression of one or more biomarkers selected from the group consisting of: alcohol dehydrogenase 1B, alcohol dehydrogenase 1C, alcohol dehydrogenase 4 and hepatocyte nuclear factor 1B, is increased or decreased in the second sample compared to the first sample.

10. The method of claim 1, wherein the first sample is a urine sample or a biopsy sample.

11. The method of claim 7, wherein the second sample is a urine sample or a biopsy sample.

12. The method of claim 1, wherein the assaying comprises detecting mRNA expression for the one or more biomarkers.

13. The method of claim 1, wherein the assaying comprises quantitative real-time polymerase chain reaction, reverse-transcription polymerase chain reaction, DASL, in-situ hybridization, Northern blot, nuclease protection and microarray analysis.

14. The method of claim 1, wherein the assaying comprises immunoassay.

15. The method of claim 1, wherein the assaying comprises spectrometry.

16. The method of claim 1, wherein the first biological sample comprises epithelial cells of the prostate gland and/or stromal cells of the prostate gland.

17. The method of claim 7, wherein the second biological sample comprises epithelial cells of the prostate gland and/or stromal cells of the prostate gland.

18. The method of claim 1, wherein the hyperplastic disease is benign prostatic hyperplasia.

19. The method of claim 1, wherein the neoplastic disease is prostatic adenocarcinoma.

20. The method of claim 1, wherein the neoplastic disease is low-grade prostatic adenocarcinoma.

21. The method of claim 1, wherein the one or more Ancestry Informative markers indicative of African descent is listed in Table III or Table IV.

22. A kit for aiding in detection, assessment and treatment of a proliferative disorder of the prostate gland in a subject, comprising:
at least one reagent for assay of alcohol dehydrogenase 1B, alcohol dehydrogenase 1C, alcohol dehydrogenase 4, hepatocyte nuclear factor 1B; and
at least one reagent for assay of prostate specific antigen or androgen receptor.

23. A kit for aiding in detection, assessment and treatment of a proliferative disorder of the prostate gland in a subject, comprising:
at least one reagent for assay of alcohol dehydrogenase 4; and
a questionnaire form for self-identification of the subject's race as of African descent or non-African descent.
